SEQUENCE LISTING (51 sequences)

SEQ ID NO: 1 - DNA, 1212 bp, Artificial Sequence
  Description of Artificial Sequence Synthetic polynucleotide
atgggctttaccctgaaaaccaccgctatcgtgtctgcggcagcccttcttgctggatttggacctccaccgcgtgcagccgaactgccacctggtcgcttggccacgaccgaggactatttcgcacaacaggccaaacaagcggttactccggatgtgatggctcaactggcgtacatgaactatattgactttatcagccccttctattctcgcggttgtagctttgaggcttgggaactgaagcataccccacagcgcgtgattaagtacagcatcgcgttttacgcttatggcctggcaagtgtggcgctgattgatccgaaactgcgtgcgttagccggtcatgatctcgacattgcggtgtcgaaaatgaagtgcaaacgggtatggggcgattgggaggaagatgggttcggtaccgatccgatcgagaaagagaacatcatgtacaaaggccatttaaacctgatgtatgggttgtaccagctcgtaacaggcagtcgtcgctatgaagccgaacacgcacatctcacccgcatcattcacgatgagattgcggcgaatccttttgcgggcattgtgtgtgaaccggataattacttcgttcagtgcaattcggtggcgtatttatccttgtgggtctatgaccggctgcatggtactgattaccgtgctgcaacacgcgcatggctggacttcatccagaaagacctgattgacccggaacgtggtgcgttctacctgtcatatcaccccgaatctggcgcagttaagccgtggattagcgcgtatacgacagcctggacgttagcgatggtacacggaatggacccggcgttttccgaacgctattatccgcgctttaaacagaccttcgtcgaagtctacgacgaaggccgtaaagcccgtgttcgcgaaactgccgggacggatgatgccgatggtggcgttggtctggcatccgcgtttacgctgcttctggcacgcgagatgggcgatcagcaactgttcgatcagttacttaaccacttggaaccgcccgccaaaccgagcattgtctcagctagtctgcgctatgaacatccggggtcgttgctcttcgatgaactgctgtttctggcaaaagtgcatgcgggctttggtgccctgttacgtatgccacctccggctgccaaactggcgggcaaacatcatcaccatcaccattaa

SEQ ID NO: 2 - DNA, 1194 bp, Artificial Sequence
  Description of Artificial Sequence Synthetic polynucleotide
atgggctttaccctgaaaaccaccgctatcgtgtctgcggcagcccttcttgctggatttggacctccaccgcgtgcagccgaactgccacctggtcgcttggccacgaccgaggactatttcgcacaacaggccaaacaagcggttactccggatgtgatggctcaactggcgtacatgaactatattgactttatcagccccttctattctcgcggttgtagctttgaggcttgggaactgaagcataccccacagcgcgtgattaagtacagcatcgcgttttacgcttatggcctggcaagtgtggcgctgattgatccgaaactgcgtgcgttagccggtcatgatctcgacattgcggtgtcgaaaatgaagtgcaaacgggtatggggcgattgggaggaagatgggttcggtaccgatccgatcgagaaagagaacatcatgtacaaaggccatttaaacctgatgtatgggttgtaccagctcgtaacaggcagtcgtcgctatgaagccgaacacgcacatctcacccgcatcattcacgatgagattgcggcgaatccttttgcgggcattgtgtgtgaaccggataattacttcgttcagtgcaattcggtggcgtatttatccttgtgggtctatgaccggctgcatggtactgattaccgtgctgcaacacgcgcatggctggacttcatccagaaagacctgattgacccggaacgtggtgcgttctacctgtcatatcaccccgaatctggcgcagttaagccgtggattagcgcgtatacgacagcctggacgttagcgatggtacacggaatggacccggcgttttccgaacgctattatccgcgctttaaacagaccttcgtcgaagtctacgacgaaggccgtaaagcccgtgttcgcgaaactgccgggacggatgatgccgatggtggcgttggtctggcatccgcgtttacgctgcttctggcacgcgagatgggcgatcagcaactgttcgatcagttacttaaccacttggaaccgcccgccaaaccgagcattgtctcagctagtctgcgctatgaacatccggggtcgttgctcttcgatgaactgctgtttctggcaaaagtgcatgcgggctttggtgccctgttacgtatgccacctccggctgccaaactggcgggcaaataa

SEQ ID NO: 3 - DNA, 1119 bp, Artificial Sequence
  Description of Artificial Sequence Synthetic polynucleotide
atggccgaactgccacctggtcgcttggccacgaccgaggactatttcgcacaacaggccaaacaagcggttactccggatgtgatggctcaactggcgtacatgaactatattgactttatcagccccttctattctcgcggttgtagctttgaggcttgggaactgaagcataccccacagcgcgtgattaagtacagcatcgcgttttacgcttatggcctggcaagtgtggcgctgattgatccgaaactgcgtgcgttagccggtcatgatctcgacattgcggtgtcgaaaatgaagtgcaaacgggtatggggcgattgggaggaagatgggttcggtaccgatccgatcgagaaagagaacatcatgtacaaaggccatttaaacctgatgtatgggttgtaccagctcgtaacaggcagtcgtcgctatgaagccgaacacgcacatctcacccgcatcattcacgatgagattgcggcgaatccttttgcgggcattgtgtgtgaaccggataattacttcgttcagtgcaattcggtggcgtatttatccttgtgggtctatgaccggctgcatggtactgattaccgtgctgcaacacgcgcatggctggacttcatccagaaagacctgattgacccggaacgtggtgcgttctacctgtcatatcaccccgaatctggcgcagttaagccgtggattagcgcgtatacgacagcctggacgttagcgatggtacacggaatggacccggcgttttccgaacgctattatccgcgctttaaacagaccttcgtcgaagtctacgacgaaggccgtaaagcccgtgttcgcgaaactgccgggacggatgatgccgatggtggcgttggtctggcatccgcgtttacgctgcttctggcacgcgagatgggcgatcagcaactgttcgatcagttacttaaccacttggaaccgcccgccaaaccgagcattgtctcagctagtctgcgctatgaacatccggggtcgttgctcttcgatgaactgctgtttctggcaaaagtgcatgcgggctttggtgccctgttacgtatgccacctccggctgccaaactggcgggcaaataa

SEQ ID NO: 4 - DNA, 1686 bp, Artificial Sequence
  Description of Artificial Sequence Synthetic polynucleotide
atggcaaccgcaaccattaccagcacccagaccaataatggcaccctggaactgcgtggtgaagcaccgagcaaactgccgaaaaccctgcctgcagattttattgaacgttttccgaaactgagccgtgaacaggcaggtcatctgcgtcattttcataatctggcaacccagaaagatggcgaatggaaacacatgggtagccaagaaccgggtcaagaatggctggatgcatatcgttatcagctggcaaccatggcatatgcagccggtgcagcacattatcatcatctgcctgcactgcgtagcacctttaaaagcctgctggaaagcctgattcataaaatgctgctgcgtgatgtttggggttattggtttctgaccagccatagcggtattatggttgatccggatattaaagaactgcgtaaaccgtgggcagatccggttgttcgtgaaaacattatgtatagtggccatctgctgctgatggttagcctgcatgaaatgctgtttcatgaaggtcgttttgatgatgaaggtagcattgcctttaattggaacccgattttttggggtatgggtccggaacgtttttgttatacccgtaaaacactgcaagaagcaattctgcgcgaaatggaacgtgaaaattggctgggtgtttgttgtgaaccgaatagcatttttgttgtgtgcaatcagtttccgctgattgccctgcgttataatgatgtgcgtgataaaattgatctgagtccgggtgttctggaaaaatatcaggcagcatggaaaagcaaaggtatgattagtgatgatggcctgattgtggattggtatagcccgaaacagaatcgtaccaaaccgcctagcgatattgcatttaccagctgggcactggcatttatgaatagctggaacccggattttgcacgtcgtaccagcaaagatattgcaattggttatctggccaaaagccatgaagatcatgtttttgttccggatcctgaagtgagctttaaaatccgtgaactggttgcaagcgaacgtctggatccgatggatccggcaacctatgcacgtgccgcaaaagcagttgcagaacagaatctgcctgcaagcggttttccgtttacaaaaccgcattttgcatatgccgcaatgtgggccagcgaactgggtgatcctgaacatctggatggtctgctggcctatgcagatgttaaaatgaatccgacctgggaagatggtggtctgttttatggtggtagcggtaaaagcgaagcagcaagcggtgttgatgttattagcggtaatgcagcagttgcgtatgcacgttttaatgtgccggatggtcagcgtaccatgtatgaaaaaccgtgggatgcagaacattttgcaaccgttccgtttgtgaaaaatgttgatctggcaagtggtgtggattttctgcgtggtagctgggatgaagaactgcaggcactggccgttaccctgcgtagttgggatggcaccaataaaagcgttcagccgcagtttaccggtctgcaagagggtaattatggtatttatcagaatggtgtgctgcaccagaccgaagaggttaaaagccgtgatgatgtgattgcatttggtctgcaggttagcggtgatgaagtggatctggttctggttcgtagccattaa

SEQ ID NO: 5 - DNA, 1359 bp, Artificial Sequence
  Description of Artificial Sequence Synthetic polynucleotide
atggcaagccagaccgcaaccaccaccgcagcaccgggtagcattccgctgagcacaccgggtccgctgccgattgcactgccgagccatattctgagcaaatttccggcactgacaccggcacaggcaggtcatctgcgtcattttcataatctggcaacccagctggatggtgaatggcgtcatatgggtgcacaggatccgggtcaagaatggctggatgcatatcgttatcagctggcaaccatggcatatgcagccggtgcagcacattatcatcgtctgcctgcactgcgtagcgtttttcgtgttctgctggaacagctgattcataaaatgctgcgtcgtgaagtttggggttattggtatctgaccagccagagcggtcgttttgttgatccggatattgaagaactgcgtaaaccgtggtcagatccgattaaacgtgaaaacattatgtacagtggccatctgctgctgatggttagcctgcatgcaatgctgtttgatgatgataaatatgatcagccggatgccctggtttttgattggaatccgattttttggggtatgggtccggaaaaattctgttatagccgtagcagcctgcagaaagcaattctggatgaaatggaacgtaccaattggatgggtgtttgttgtgaaccgaatagcgtgtttgtggtttgtaatcagtttccgctgattgccattcgctataacgatgttcgtaatggcaccaatgttattgatggtgtgctggataaatatcgtgcagcatgggatagccgtaatggttttacccagggtggtgatcagatggttgcatggtggcgtccgaaacagcaggattttgttccgggtagcagcattggttttagcagctgggcaagcgcatttatgaatgcatggaatccgagctattgtcatgcaatgtatccgagctttgcactgggtaatctgacccgtcatccgagcggtcgtgttaatctgaatccgcctgcagttgcagcagaaattcgtgcactggttcatgatgatccggcaaccgatccgcatgcaccggcaaccctggatcgtgcacgtggtcgtgcagccgaaaaagcagcagcagccgcagcacgtcagcagcaacagcctccgggtccgcctaaaccgcctgcaagtccggaatttggttatgttgttaaatgggttattagtccggtggtgaaaaatctgcctgcaggtctgtatggtatttatgaaggtggtaaactggttcagacccgtagcaccggtggtggtgatggtggtattgatctggaactgcaggttggcggtgatgaactggatgttgttctgctgaaacagaaataa

SEQ ID NO: 6 - DNA, 1935 bp, Artificial Sequence
  Description of Artificial Sequence Synthetic polynucleotide
atggaaagcacccgtatgctgcgtcagccgattcagctgctgcagggtcataaaggtccggttaccgcaagccgtcatcgtcgtaatgcagttgtttatgcactgctgtgtctgctggcactgctgccggttgccaccggtcagagcgcagcatggcaggcagcaggtctgggtctgtttatgcctggtgcaggttttctggcactgggtggtgcatgggctctgctgtttccgctgaccgtttttgttttttggctggcagttattgcatggttttggagcggtatggttgttgcaccgctgaccctgtggctgggcaccgctgcactggcaggatggctggctggtgaagcaatttggcctccggcagtttatctggctccggcagccgcagcagcaacctttctgttttttcagtatcgtggtgcaaaacgtcgtgcaaaagatcgtgaacatttcaaatttcgccagagcttttttgcagaaagcctggccgaagttcatcagcgtgcagcaaccgaaccggaaccgggtgaacgtgaactgacaccggatcagctgcagggcgttcgttatctgctggaactggcgctgcagccggtgggtcagtataaaggttataccattatcgatcagtttcagcctgcagcactgcgttatcagctgaaccatattggttttgcactgggtatggtacagggtcactataccccgaattttcagggttatctgggtcaggcacagcgtaatgttattgatacctatcgtgaacgtaaagtgtggggttattgggtttatgaaagcatgtggggtcactttaacttcagcgattttgatccggcacgcaaagataatatcatgctgaccggttggtatggtatgcatgttggccagtatatgctgaatgccggtgatacccgttatagccagcctggtagcctgagctttcgtctgaatgataagacctgttatcatcatgatatccatagcattaatcagagcgtgcgtgaaaactttcagagcagcgatttttgtctgtatccgtgtgaaccgaattgggtttatccggtttgtaatatgtatggtatgagcagcctggcagtttatgataccctgtttgaacgtcgtgataccgcacaggttctgccgaaatggctgcatatgctggataccgaatttaccgatcagaaaggtagcctggttggtctgcgtagctattggaccggtctggaaatgccgttttataccggtgaagcaggttttgcatttttcgccaacatttttagcaccgatctggcacgtaaactgtgggcagttggtcgtaaagaactgagcatgtgtctgacccaggatgcagaaggtcagacccgtctgacactgccgaaagaagcactggccttttttgataccattgatgcaggtaattatcgccctggtaaactgtttgcatatgttgcagttcagatgtgtgcacgtgaatttggtgatgatgaactggcagaagcagcacgtcgtagcatggatcaggattgtggtccggttgttgaaaatggtgttgcacgttataccaaaggtagcaccctggccaatatttggggtgttgaaggtcgtctgatgcgtaccggtgattttcgtaatagctttgttaaaggtccgcctagcagcgtgtttgatggtccgctgctgggtgatgcccgttatccggaaattctggttgcaaaagcatttagtcgcggtgatgatctggaactggtgctgtatccgggtgccggtgatggtcctcagaccctgggttttgaacgcctgaaaccgggtgttcgttatgttgtggaaggtgcagcaagcggtgaatttacagcagatgcagatggtcgtgcaagcctggccgttaccctgagcggtcgtaccgcactgcatattaaaccgggtcattaa

SEQ ID NO: 7 - DNA, 1953 bp, Artificial Sequence
  Description of Artificial Sequence Synthetic polynucleotide
atggcaccgagcaccatgaccaccaccaccgaaaccaccaaaaccaatggtgttaatcatctggatgcagcagaactgccgagcaaaattgcaccgagtagcaaatgggttgataccagcgatagcattaaagcagatccgagcacctcagttaaagcaagtgatggtccggttggtgattttggttggggtccgtggaaaattcaggttccggttgaatatacctttctgagcctggcaggtctgtggtcatttcataatctggaaagcaccagctatcgtgcagcagcactgggttttctgtttccgggtgcaggttttaccgcagttgcaagcccgaccgcagtggcagcatttctgctgaccctggttctgattccggttagcatttttgtttggtttgcaatgggtggtattgcatttccgattgcactgtggattggtagcagctttatggcaggtcgtctggcacaggataccctgtttgaacagagcgcagcactgtgggcactgggctgttttagcggtattacctggctgatgaataatgcaagcagcctgaatgcagcaggttatagcaaagcacaagaacgcaacaaatatctggttcaggcagttgaagaacaaatggcagatgcagcaccggcaccgcagagcggtgatcgtgaactgagtctggaaaccctgcgtcatgttcagcatatgattgaacgtggtctgagtccgcgtgatgattttagctttcatgatgtgattgatcagtttcagaccggtgccattcgttatcagctgtatggcaccattgatgcactgagcctgtatcagtgtcattatgttccgggttttcatggctatctgagcaaagcatgtcagaacgcaattgaaaaaagcctgcagaaacgcattatgagctattggaaatgggaaagcatctttggtcgttttaccctgagcgattgggatccgatcaaaaaagataacattatggtgaccggttatctgagcgcagccattggtctgtacggtcaggcaagcggtgatcgtcagtataacaaaaaagatgccctggaattcgtgatcgatgatggcaaacactataaaaccaattatgaaggtctggccgatgccctgtttaataacatgaccgaaaatccgtattgtctgtatccgtgtgaaccgaattggacctatagcctgtgtaatctgaccggtatggcaggtctggttattagcgatcgtctgctgggtcgtgatctgggtgttaaactgcgtaatcgttttgaacgtagcctggaagaggaatttaccgaatgtgatggtcgtattctgccgattcgtagcgaatttacaggtctgaccctgcctggtctgtgtggcaccctgaccgattgtattaatgcaatgctgctgaccgcatatctgccgcatctggcacatcgtaattgggcaatgattcgtaaagagttcctgaaatacgataaaaacggtcagctggaagttcgtagcctgaaaggtgcagataaaatggatccgcgtaattatcgtgcaagcgaaggtccgctgcgtgcatttattgcagcaaccgcagcagaatttggcaatgaaaaaattcgcaaagaagcactgcatcagctggataatacctattttccggttgaagcaaccaaaagcggtagcctgcgtaataaacgtagcggtcctctgctggaagaggcaccgtttccggatgttctggttgcaaaagcatatagcaatgatggtaaacagctggatctggtgctgtataatggtgcagaaccgggtacatttgaactgggctttgaacgtctggttccgggtaaagaatattcactgagcaccggtggtagcgttaaagcaaataacaaaggtaaagccaccgttaaagttagcgtgaaaggtcgtacccagattattctgaaaccggttgtttaa

SEQ ID NO: 8 - DNA, 1920 bp, Artificial Sequence
  Description of Artificial Sequence Synthetic polynucleotide
atgagcgcaccgaccattgaaagcgaacagagcaccgatattggttatgtttttgaagttccggatcgtccgaatggtccggcagttcgtcgtctgctgcgtcgtagcggtgcactgctgggtgcagttggcaccgttgcaaccctgaccgcatggcgtagcaaacgtccgcgtgttcgtgcaattgccctgggtctgctggcaccgggtggtggtcagctgtatacccgtagtccgctgcgttttgttgcaaccgttgcaggtttttttgcaagcctggttgcatggtttggtagcggtaacattattgcaccggttctggtttggctgaccgcagcaaccaatgcaggtagccatgcagccggtggtcgtcgtacctggaatggtgcacgtcgtgttattccgaccgcagttgccggtgcagcagcagccggtattgttgcccgtcgtcgtgcatttcatgcagcacaggcacgtggtcgtacccgtgcagaatatctgaccaccgcaccgcgtctggatccgcagccgaaagatgcagcaagcgaagaactgagcccgaccgatctggcagttctgcgtagcctgctggatcgtgcactgcagccgctggaaaattttgatggttttgatcgcattgatcagtttcagaccagcgcaatgcgttatcagtgcaattttatgcagtatgcactggcaaccgcacagctgcatgcaaccccgagctttcatggttatctgagcgcagcacagcgtaatctgattgataaactgaccctgcctgcagtttggcgttattgggcatttgaacagacctggggtaatctgagcctggattgggatccgatgaaacgtgataacattatgctgagcggttatctgggtatgatgctgggagcatatgaaagcaataccggtgatgatcgttatcgtcgttcaggtgccctgccgtttcgtctggggaaacgtgattggccgtatacccatgatatggttagcgcagcagttcatgataatatgcagcgtagcggtatgaccctgtttccgtgtgaaccgaattggatttatagcgcatgtaatatgcctgcaattagcagcctgatgatgagcgatcgtctgcatggcacccgttatattgaaagcgttggtgaagattttcgtcgtcgcctgcatggtgaatttatcacaccggatggtcgtattaccgcaattcgtagcagccgtctgggtgttaccattccgatgctgaccagcaccatggcagattgtggtctggcaagcatgctgcatgcatttgatccggaactggcacagcgttgttggaccattgcacgtcgcgaatttattgataccaccggtccggaacctgttattgttctgcgtggttgggatgcaattgataccggtaattatcgtaaaaccaccctgggtgcagttgcaccggttatgtgggcagcagcagaaatgggtgataccgatctggttgcacagctgaccaccacactggaacgtcatgcacagccgaccgaaaccggtggtgcacgttggtatgcagaactgagcaccaatatgaatgcaatggcagcactggcacgttttaatcctccgggtggccagcgtgcactgattagcgcaggtccgggtacacagattctgacaggtccggttctggatgatgttgtttatccggaagttctggttgcgagcgcacgtaccgatggtgccgatctgcgtctggttctgcgtccgggtgccggtgcagcacgtgttagcattggtgttcgtcatctgcatcctggtggtcgttatcgtgttaatggtgccgttgatagcgaagttaccgcagataatcagggtcgtagccatctggaagttgatctgattgatcgtaccgaagttagcctgactccggcaccgtaa

SEQ ID NO: 9 - DNA, 1203 bp, Artificial Sequence
  Description of Artificial Sequence Synthetic polynucleotide
atgctgccggaacgtctgaccgcatatctgcgttattgtacccgtctggcactgcaggcaccgaatcgttgggatggttttgatctgcatgcaccggatgcacgtccgaccgcactgcgtaatcagatcttttttgttggttgtgcactggcagccctggcacgtcatccgcatgcagcacaagaggaacgtgcaatggcagttgatgccctggcagatctgaccgatcgtatgattcagcgtcgtgtttgggcagcatgggcaaccgaaaccgaacgtaccagcctgcgtccggatcctgttgatgcaggttatggcacctataccgcaccgctggcaatgctgtttggtctgcagggtgttctgggtggtcaggttcgttatggtgaagatccgtttaccctgcgttggagcgcagatgttcgtagctgttataccgttcgtgaactgattgcagcactggcaaaacagagccaggatagtccggaaggtgcaattcgttgtgaaggtgatctggcaaccccgagcgccatggcagcgctggtttgggcactgcgtctgcatgatctggcctatgcaaccgaatatggcaccagcggcaccacctggctgaaaaccctgggtgaacgtatggcaattcgtggtccgcgtctgtttaatcgtcataccctggcagcaggttggaatattgcaaatcgtcgtgcaagcggtagtgcagatggtctggaagatgcatgggcactggccctgagcgctccgctggatcgcgaactgatcgcaggcttggcagaacgttattgggcaggcgcagataaactgcgtgaacgtggtgatgcactgagcctgggttttagctatctgctggcagttgaactgggtgaaacccagctggcagcaagcctgctggcaagcgcagaacagcgttttggttttgatgaagatgatgaacagggtcgtcgcattaaagatagtccggttaccccgtgggttaccgcactgtttgcaattggtgaagccggtggtatggcacgtctgctggaagcagcactgcctccgctgccgcagccggaaattcctgcaccgggttggcctgaatggccagagtggccagaatggcctccgctggatctggcagaaccgcagctggaacaggatcaggccgaagaacgtcgtgatcaggcagcagaagaagaaggttgttaa

SEQ ID NO: 10 - DNA, 1137 bp, Artificial Sequence
  Description of Artificial Sequence Synthetic polynucleotide
atggccgaactgccacctggtcgcttggccacgaccgaggactatttcgcacaacaggccaaacaagcggttactccggatgtgatggctcaactggcgtacatgaactatattgactttatcagccccttctattctcgcggttgtagctttgaggcttgggaactgaagcataccccacagcgcgtgattaagtacagcatcgcgttttacgcttatggcctggcaagtgtggcgctgattgatccgaaactgcgtgcgttagccggtcatgatctcgacattgcggtgtcgaaaatgaagtgcaaacgggtatggggcgattgggaggaagatgggttcggtaccgatccgatcgagaaagagaacatcatgtacaaaggccatttaaacctgatgtatgggttgtaccagctcgtaacaggcagtcgtcgctatgaagccgaacacgcacatctcacccgcatcattcacgatgagattgcggcgaatccttttgcgggcattgtgtgtgaaccggataattacttcgttcagtgcaattcggtggcgtatttatccttgtgggtctatgaccggctgcatggtactgattaccgtgctgcaacacgcgcatggctggacttcatccagaaagacctgattgacccggaacgtggtgcgttctacctgtcatatcaccccgaatctggcgcagttaagccgtggattagcgcgtatacgacagcctggacgttagcgatggtacacggaatggacccggcgttttccgaacgctattatccgcgctttaaacagaccttcgtcgaagtctacgacgaaggccgtaaagcccgtgttcgcgaaactgccgggacggatgatgccgatggtggcgttggtctggcatccgcgtttacgctgcttctggcacgcgagatgggcgatcagcaactgttcgatcagttacttaaccacttggaaccgcccgccaaaccgagcattgtctcagctagtctgcgctatgaacatccggggtcgttgctcttcgatgaactgctgtttctggcaaaagtgcatgcgggctttggtgccctgttacgtatgccacctccggctgccaaactggcgggcaaacatcatcaccatcaccattaa

SEQ ID NO: 11 - protein, 397 aa, Castellaniella defragrans
MRFTLKTTAIVSAAALLAGFGPPPRAAELPPGRLATTEDYFAQQAKQAVTPDVMAQLAYMNYIDFISPFYSRGCSFEAWELKHTPQRVIKYSIAFYAYGLASVALIDPKLRALAGHDLDIAVSKMKCKRVWGDWEEDGFGTDPIEKENIMYKGHLNLMYGLYQLVTGSRRYEAEHAHLTRIIHDEIAANPFAGIVCEPDNYFVQCNSVAYLSLWVYDRLHGTDYRAATRAWLDFIQKDLIDPERGAFYLSYHPESGAVKPWISAYTTAWTLAMVHGMDPAFSERYYPRFKQTFVEVYDEGRKARVRETAGTDDADGGVGLASAFTLLLAREMGDQQLFDQLLNHLEPPAKPSIVSASLRYEHPGSLLFDELLFLAKVHAGFGALLRMPPPAAKLAGK

SEQ ID NO: 12 - DNA, 1137 bp, Artificial Sequence; identical to SEQ ID NO: 10
  Description of Artificial Sequence Synthetic polynucleotide

SEQ ID NO: 13 - protein, 378 aa, Artificial Sequence
  Description of Artificial Sequence Synthetic polypeptide
MAELPPGRLATTEDYFAQQAKQAVTPDVMAQLAYMNYIDFISPFYSRGCSFEAWELKHTPQRVIKYSIAFYAYGLASVALIDPKLRALAGHDLDIAVSKMKCKRVWGDWEEDGFGTDPIEKENIMYKGHLNLMYGLYQLVTGSRRYEAEHAHLTRIIHDEIAANPFAGIVCEPDNYFVQCNSVAYLSLWVYDRLHGTDYRAATRAWLDFIQKDLIDPERGAFYLSYHPESGAVKPWISAYTTAWTLAMVHGMDPAFSERYYPRFKQTFVEVYDEGRKARVRETAGTDDADGGVGLASAFTLLLAREMGDQQLFDQLLNHLEPPAKPSIVSASLRYEHPGSLLFDELLFLAKVHAGFGALLRMPPPAAKLAGKHHHHHH

SEQ ID NO: 14 - protein, 378 aa, Artificial Sequence
  Description of Artificial Sequence Synthetic polypeptide
MAELPPGRLATTEDYFAQQAKQAVTPDVMAQLAYMNYIDFISPFYSRGCSFEAWELKATPQRVIKYSIAFYAYGLASVALIDPKLRALAGHDLDIAVSKMKCKRVWGDWEEDGFGTDPIEKENIMYKGHLNLMYGLYQLVTGSRRYEAEHAHLTRIIHDEIAANPFAGIVCEPDNYFVQCNSVAYLSLWVYDRLHGTDYRAATRAWLDFIQKDLIDPERGAFYLSYHPESGAVKPWISAYTTAWTLAMVHGMDPAFSERYYPRFKQTFVEVYDEGRKARVRETAGTDDADGGVGLASAFTLLLAREMGDQQLFDQLLNHLEPPAKPSIVSASLRYEHPGSLLFDELLFLAKVHAGFGALLRMPPPAAKLAGKHHHHHH

SEQ ID NO: 15 - protein, 378 aa, Artificial Sequence
  Description of Artificial Sequence Synthetic polypeptide
MAELPPGRLATTEDYFAQQAKQAVTPDVMAQLAYMNYIDFISPFYSRGCSFEAWELKHTPQRVIKYSIAFYAYGLASVALIDPKLRALAGHDLDIAVSKMKCKRVWGDWEEDGFGTDPIEKENIMYKGHLNLMYGLYQLVTGSRRYEAEHAHLTRIIHDEIAANPFAGIVCEPDNYFVQCNSVAYLSLWVYDRLHGTDYRAATRAWLDFIQKDLIDPERGAFYLSYAPESGAVKPWISAYTTAWTLAMVHGMDPAFSERYYPRFKQTFVEVYDEGRKARVRETAGTDDADGGVGLASAFTLLLAREMGDQQLFDQLLNHLEPPAKPSIVSASLRYEHPGSLLFDELLFLAKVHAGFGALLRMPPPAAKLAGKHHHHHH

SEQ ID NO: 16 - protein, 378 aa, Artificial Sequence
  Description of Artificial Sequence Synthetic polypeptide
MAELPPGRLATTEDYFAQQAKQAVTPDVMAQLAYMNYIDFISPFYSRGCSFEAWELKATPQRVIKYSIAFYAYGLASVALIDPKLRALAGHDLDIAVSKMKCKRVWGDWEEDGFGTDPIEKENIMYKGHLNLMYGLYQLVTGSSRYEAEHAHLTRIIHDEIAANPFAGIVCEPDNYFVQCNSVAYLSLWVYDRLHGTDYRAATRAWLDFIQKDLIDPERGAFYLSYHPESGAVKPWISAYTTAWTLAMVHGMDPAFSERYYPRFKQTFVEVYDEGRKARVRETAGTDDADGGVGLASAFTLLLAREMGDQQLFDQLLNHLEPPAKPSIVSASLRYEHPGSLLFDELLFLAKVHAGFGALLRMPPPAAKLAGKHHHHHH

SEQ ID NO: 17 - protein, 378 aa, Artificial Sequence
  Description of Artificial Sequence Synthetic polypeptide
MAELPPGRLATTEDYFAQQAKQAVTPDVMAQLAYMNYIDFISPFYSRGCSFEAWELKATPQRVIKYSIAFYAYGLASVALIDPKLRALAGHDLDIAVSKMKCKRVWGDWEEDGFGTDPIEKENIMYKGHLNLMYGLYQLVTGSGRYEAEHAHLTRIIHDEIAANPFAGIVCEPDNYFVQCNSVAYLSLWVYDRLHGTDYRAATRAWLDFIQKDLIDPERGAFYLSYHPESGAVKPWISAYTTAWTLAMVHGMDPAFSERYYPRFKQTFVEVYDEGRKARVRETAGTDDADGGVGLASAFTLLLAREMGDQQLFDQLLNHLEPPAKPSIVSASLRYEHPGSLLFDELLFLAKVHAGFGALLRMPPPAAKLAGKHHHHHH

SEQ ID NO: 18 - protein, 378 aa, Artificial Sequence
  Description of Artificial Sequence Synthetic polypeptide
MAELPPGRLATTEDYFAQQAKQAVTPDVMAQLAYMNYIDFISPFYSRGCSFEAWELKATPQRVIKYSIAFYAYGLASVALIDPKLRALAGHDLDIAVSKMKCKRVWGDWEEDGFGTDPIEKENIMYKGHLNLMYGLYQLVTGSRRYEAEHAHLTRIIHDECAANPFAGIVCEPDNYFVQCNSVAYLSLWVYDRLHGTDYRAATRAWLDFIQKDLIDPERGAFYLSYHPESGAVKPWISAYTTAWTLAMVHGMDPAFSERYYPRFKQTFVEVYDEGRKARVRETAGTDDADGGVGLASAFTLLLAREMGDQQLFDQLLNHLEPPAKPSIVSASLRYEHPGSLLFDELLFLAKVHAGFGALLRMPPPAAKLAGKHHHHHH

SEQ ID NO: 19 - protein, 378 aa, Artificial Sequence
  Description of Artificial Sequence Synthetic polypeptide
MAELPPGRLATTEDYFAQQAKQAVTPDVMAQLAYMNYIDFISPFYSRGCSFEAWELKATPQRVIKYSIAFYAYGLASVALIDPKLRALAGHDLDIAVSKMKCKRVWGDWEEDGFGTDPIEKENIMYKGHLNLMYGLYQLVTGSRRYEAEHAHLTRIIHDEIAANPFAGIVCEPDNYFVQCNSVAYLSLWVYDRLHGTDYRAATRAWLDFIQKDLIDPERGAFYLSYHPESGAVKPWISAYTTAWTLAMVHGMDPAFSERYYPRFKQTFVEVYDEGRKARVRETAGTDDADGGVGLASAFTLLLAREMGDQQLFDQLLNHLEPPAKPSIVSASLSYEHPGSLLFDELLFLAKVHAGFGALLRMPPPAAKLAGKHHHHHH

SEQ ID NO: 20 - protein, 378 aa, Artificial Sequence
  Description of Artificial Sequence Synthetic polypeptide
MAELPPGRLATTEDYFAQQAKQAVTPDVMAQLAYMNYIDFISPFYSRGCSFEAWELKATPQRVIKYSIAFYAYGLASVALIDPKLRALAGHDLDIAVSKMKCKRVWGDWEEDGFGTDPIEKENIMYKGHLNLMYGLYQLVTGSRRYEAEHAHLTRIIHDEIAANPFAGIVCEPDNYFVQCNSVAYLSLWVYDRLHGTDYRAATRAWLDFIQKDLIDPERGAFYLSYHPESGAVKPWISAYTTAWTLAMVHGMDPAFSERYYPRFKQTFVEVYDEGRKARVRETAGTDDADGGVGLASAFTLLLAREMGDQQLFDQLLNHLEPPAKPSIVSASLLYEHPGSLLFDELLFLAKVHAGFGALLRMPPPAAKLAGKHHHHHH

SEQ ID NO: 21 - protein, 378 aa, Artificial Sequence
  Description of Artificial Sequence Synthetic polypeptide
MAELPPGRLATTEDYFAQQAKQAVTPDVMAQLAYMNYIDFISPFYSRGCSFEAWELKHTPQRVIKYSIAFYAYGLASVALIDPKLRALAGHDLDIAVSKMKCKRVWGDWEEDGFGTDPIEKENIMYKGHLNLMYGLYQLVTGSHRYEAEHAHLTRIIHDEIAANPFAGIVCEPDNYFVQCNSVAYLSLWVYDRLHGTDYRAATRAWLDFIQKDLIDPERGAFYLSYHPESGAVKPWISAYTTAWTLAMVHGMDPAFSERYYPRFKQTFVEVYDEGRKARVRETAGTDDADGGVGLASAFTLLLAREMGDQQLFDQLLNHLEPPAKPSIVSASLRYEHPGSLLFDELLFLAKVHAGFGALLRMPPPAAKLAGKHHHHHH

SEQ ID NO: 22 - protein, 378 aa, Artificial Sequence
  Description of Artificial Sequence Synthetic polypeptide
MAELPPGRLATTEDYFAQQAKQAVTPDVMAQLAYMNYIDFISPFYSRGCSFEAWELKHTPQRVIKYSIAFYAYGLASVALIDPKLRALAGHDLDIAVSKMKCKRVWGDWEEDGFGTDPIEKENIMYKGHLNLMYGLYQLVTGSDRYEAEHAHLTRIIHDEIAANPFAGIVCEPDNYFVQCNSVAYLSLWVYDRLHGTDYRAATRAWLDFIQKDLIDPERGAFYLSYHPESGAVKPWISAYTTAWTLAMVHGMDPAFSERYYPRFKQTFVEVYDEGRKARVRETAGTDDADGGVGLASAFTLLLAREMGDQQLFDQLLNHLEPPAKPSIVSASLRYEHPGSLLFDELLFLAKVHAGFGALLRMPPPAAKLAGKHHHHHH

SEQ ID NO: 23 - protein, 378 aa, Artificial Sequence
  Description of Artificial Sequence Synthetic polypeptide
MAELPPGRLATTEDYFAQQAKQAVTPDVMAQLAYMNYIDFISPFYSRGCSFEAWELKHTPQRVIKYSIAFYAYGLASVALIDPKLRALAGHDLDIAVSKMKCKRVWGDWEEDGFGTDPIEKENIMYKGHLNLMYGLYQLVTGSRRYEAEHAHLTRIIHDEMAANPFAGIVCEPDNYFVQCNSVAYLSLWVYDRLHGTDYRAATRAWLDFIQKDLIDPERGAFYLSYHPESGAVKPWISAYTTAWTLAMVHGMDPAFSERYYPRFKQTFVEVYDEGRKARVRETAGTDDADGGVGLASAFTLLLAREMGDQQLFDQLLNHLEPPAKPSIVSASLRYEHPGSLLFDELLFLAKVHAGFGALLRMPPPAAKLAGKHHHHHH

SEQ ID NO: 24 - protein, 378 aa, Artificial Sequence
  Description of Artificial Sequence Synthetic polypeptide
MAELPPGRLATTEDYFAQQAKQAVTPDVMAQLAYMNYIDFISPFYSRGCSFEAWELKHTPQRVIKYSIAFYAYGLASVALIDPKLRALAGHDLDIAVSKMKCKRVWGDWEEDGFGTDPIEKENIMYKGHLNLMYGLYQLVTGSRRYEAEHAHLTRIIHDEIAANPFAGIVCEPDNYFVQCNSVAYLSLWVYDRLHGTDYRAATRAWLDFIQKDLIDPERGAFYLSYHPESGAVKPWISAYTTAWTLAMVHGMDPAFSERYYPRFKQTFVEVYDEGRKARVRETAGTDDADGGVGLASAFTLLLAREMGDQQLFDQLLNHLEPPAKPSIVSASLSYEHPGSLLFDELLFLAKVHAGFGALLRMPPPAAKLAGKHHHHHH

SEQ ID NO: 25 - protein, 378 aa, Artificial Sequence
  Description of Artificial Sequence Synthetic polypeptide
MAELPPGRLATTEDYFAQQAKQAVTPDVMAQLRYMNYIDFISPFYSRGCSFEAWELKHTPQRVIKYSIAFYAYGLASVALIDPKLRALAGHDLDIAVSKMKCKRVWGDWEEDGFGTDPIEKENIMYKGHLNLMYGLYQLVTGSRRYEAEHAHLTRIIHDEIAANPFAGIVCEPDNYFVQCNSVAYLSLWVYDRLHGTDYRAATRAWLDFIQKDLIDPERGAFYLSYHPESGAVKPWISAYTTAWTLAMVHGMDPAFSERYYPRFKQTFVEVYDEGRKARVRETAGTDDADGGVGLASAFTLLLAREMGDQQLFDQLLNHLEPPAKPSIVSASLRYEHPGSLLFDELLFLAKVHAGFGALLRMPPPAAKLAGKHHHHHH

SEQ ID NO: 26 - protein, 378 aa, Artificial Sequence
  Description of Artificial Sequence Synthetic polypeptide
MAELPPGRLATTEDYFAQQAKQAVTPDVMAQLAAMNYIDFISPFYSRGCSFEAWELKHTPQRVIKYSIAFYAYGLASVALIDPKLRALAGHDLDIAVSKMKCKRVWGDWEEDGFGTDPIEKENIMYKGHLNLMYGLYQLVTGSRRYEAEHAHLTRIIHDEIAANPFAGIVCEPDNYFVQCNSVAYLSLWVYDRLHGTDYRAATRAWLDFIQKDLIDPERGAFYLSYHPESGAVKPWISAYTTAWTLAMVHGMDPAFSERYYPRFKQTFVEVYDEGRKARVRETAGTDDADGGVGLASAFTLLLAREMGDQQLFDQLLNHLEPPAKPSIVSASLRYEHPGSLLFDELLFLAKVHAGFGALLRMPPPAAKLAGKHHHHHH

SEQ ID NO: 27 - protein, 378 aa, Artificial Sequence
  Description of Artificial Sequence Synthetic polypeptide
MAELPPGRLATTEDYFAQQAKQAVTPDVMAQLAYMNYIDFISPFYSRGCSFEAWELKHEPQRVIKYSIAFYAYGLASVALIDPKLRALAGHDLDIAVSKMKCKRVWGDWEEDGFGTDPIEKENIMYKGHLNLMYGLYQLVTGSRRYEAEHAHLTRIIHDEIAANPFAGIVCEPDNYFVQCNSVAYLSLWVYDRLHGTDYRAATRAWLDFIQKDLIDPERGAFYLSYHPESGAVKPWISAYTTAWTLAMVHGMDPAFSERYYPRFKQTFVEVYDEGRKARVRETAGTDDADGGVGLASAFTLLLAREMGDQQLFDQLLNHLEPPAKPSIVSASLRYEHPGSLLFDELLFLAKVHAGFGALLRMPPPAAKLAGKHHHHHH

SEQ ID NO: 28 - protein, 378 aa, Artificial Sequence
  Description of Artificial Sequence Synthetic polypeptide
MAELPPGRLATTEDYFAQQAKQAVTPDVMAQLAYMNYIDFISPFYSRGCSFEAWELKHTPQRVIKYSLAFYAYGLASVALIDPKLRALAGHDLDIAVSKMKCKRVWGDWEEDGFGTDPIEKENIMYKGHLNLMYGLYQLVTGSRRYEAEHAHLTRIIHDEIAANPFAGIVCEPDNYFVQCNSVAYLSLWVYDRLHGTDYRAATRAWLDFIQKDLIDPERGAFYLSYHPESGAVKPWISAYTTAWTLAMVHGMDPAFSERYYPRFKQTFVEVYDEGRKARVRETAGTDDADGGVGLASAFTLLLAREMGDQQLFDQLLNHLEPPAKPSIVSASLRYEHPGSLLFDELLFLAKVHAGFGALLRMPPPAAKLAGKHHHHHH

SEQ ID NO: 29 - protein, 378 aa, Artificial Sequence
  Description of Artificial Sequence Synthetic polypeptide
MAELPPGRLATTEDYFAQQAKQAVTPDVMAQLAYMNYIDFISPFYSRGCSFEAWELKHTPQRVIKYSIAFYAYGLASVALIDPALRALAGHDLDIAVSKMKCKRVWGDWEEDGFGTDPIEKENIMYKGHLNLMYGLYQLVTGSRRYEAEHAHLTRIIHDEIAANPFAGIVCEPDNYFVQCNSVAYLSLWVYDRLHGTDYRAATRAWLDFIQKDLIDPERGAFYLSYHPESGAVKPWISAYTTAWTLAMVHGMDPAFSERYYPRFKQTFVEVYDEGRKARVRETAGTDDADGGVGLASAFTLLLAREMGDQQLFDQLLNHLEPPAKPSIVSASLRYEHPGSLLFDELLFLAKVHAGFGALLRMPPPAAKLAGKHHHHHH

SEQ ID NO: 30 - protein, 378 aa, Artificial Sequence
  Description of Artificial Sequence Synthetic polypeptide
MAELPPGRLATTEDYFAQQAKQAVTPDVMAQLAYMNYIDFISPFYSRGCSFEAWELKHTPQRVIKYSIAFYAYGLASVALIDPKLRALAGHDLDIAISKMKCKRVWGDWEEDGFGTDPIEKENIMYKGHLNLMYGLYQLVTGSRRYEAEHAHLTRIIHDEIAANPFAGIVCEPDNYFVQCNSVAYLSLWVYDRLHGTDYRAATRAWLDFIQKDLIDPERGAFYLSYHPESGAVKPWISAYTTAWTLAMVHGMDPAFSERYYPRFKQTFVEVYDEGRKARVRETAGTDDADGGVGLASAFTLLLAREMGDQQLFDQLLNHLEPPAKPSIVSASLRYEHPGSLLFDELLFLAKVHAGFGALLRMPPPAAKLAGKHHHHHH

SEQ ID NO: 31 - protein, 378 aa, Artificial Sequence
  Description of Artificial Sequence Synthetic polypeptide
MAELPPGRLATTEDYFAQQAKQAVTPDVMAQLAYMNYIDFISPFYSRGCSFEAWELKHTPQRVIKYSIAFYAYGLASVALIDPKLRALAGHDLDIAVSKMKCKRVWGDWEERGFGTDPIEKENIMYKGHLNLMYGLYQLVTGSRRYEAEHAHLTRIIHDEIAANPFAGIVCEPDNYFVQCNSVAYLSLWVYDRLHGTDYRAATRAWLDFIQKDLIDPERGAFYLSYHPESGAVKPWISAYTTAWTLAMVHGMDPAFSERYYPRFKQTFVEVYDEGRKARVRETAGTDDADGGVGLASAFTLLLAREMGDQQLFDQLLNHLEPPAKPSIVSASLRYEHPGSLLFDELLFLAKVHAGFGALLRMPPPAAKLAGKHHHHHH

SEQ ID NO: 32 - protein, 378 aa, Artificial Sequence
  Description of Artificial Sequence Synthetic polypeptide
MAELPPGRLATTEDYFAQQAKQAVTPDVMAQLAYMNYIDFISPFYSRGCSFEAWELKHTPQRVIKYSIAFYAYGLASVALIDPKLRALAGHDLDIAVSKMKCKRVWGDWEEDGFGTDPIEKENIMYKGHLNLMYGLYQLVTGDRRYEAEHAHLTRIIHDEIAANPFAGIVCEPDNYFVQCNSVAYLSLWVYDRLHGTDYRAATRAWLDFIQKDLIDPERGAFYLSYHPESGAVKPWISAYTTAWTLAMVHGMDPAFSERYYPRFKQTFVEVYDEGRKARVRETAGTDDADGGVGLASAFTLLLAREMGDQQLFDQLLNHLEPPAKPSIVSASLRYEHPGSLLFDELLFLAKVHAGFGALLRMPPPAAKLAGKHHHHHH

SEQ ID NO: 33 - protein, 378 aa, Artificial Sequence; identical to SEQ ID NO: 22
  Description of Artificial Sequence Synthetic polypeptide

SEQ ID NO: 34 - protein, 378 aa, Artificial Sequence
  Description of Artificial Sequence Synthetic polypeptide
MAELPPGRLATTEDYFAQQAKQAVTPDVMAQLAYMNYIDFISPFYSRGCSFEAWELKHTPQRVIKYSIAFYAYGLASVALIDPKLRALAGHDLDIAVSKMKCKRVWGDWEEDGFGTDPIEKENIMYKGHLNLMYGLYQLVTGSRRYEAEHAHLTRIIHDEIAANPFAGIVCEPNNYFVQCNSVAYLSLWVYDRLHGTDYRAATRAWLDFIQKDLIDPERGAFYLSYHPESGAVKPWISAYTTAWTLAMVHGMDPAFSERYYPRFKQTFVEVYDEGRKARVRETAGTDDADGGVGLASAFTLLLAREMGDQQLFDQLLNHLEPPAKPSIVSASLRYEHPGSLLFDELLFLAKVHAGFGALLRMPPPAAKLAGKHHHHHH

SEQ ID NO: 35 - protein, 378 aa, Artificial Sequence
  Description of Artificial Sequence Synthetic polypeptide
MAELPPGRLATTEDYFAQQAKQAVTPDVMAQLAYMNYIDFISPFYSRGCSFEAWELKHTPQRVIKYSIAFYAYGLASVALIDPKLRALAGHDLDIAVSKMKCKRVWGDWEEDGFGTDPIEKENIMYKGHLNLMYGLYQLVTGSRRYEAEHAHLTRIIHDEIAANPFAGIVCEPDNYFVQCNSVAYLSLWVYDRLHGTDYRAATREWLDFIQKDLIDPERGAFYLSYHPESGAVKPWISAYTTAWTLAMVHGMDPAFSERYYPRFKQTFVEVYDEGRKARVRETAGTDDADGGVGLASAFTLLLAREMGDQQLFDQLLNHLEPPAKPSIVSASLRYEHPGSLLFDELLFLAKVHAGFGALLRMPPPAAKLAGKHHHHHH

SEQ ID NO: 36 - protein, 378 aa, Artificial Sequence
  Description of Artificial Sequence Synthetic polypeptide
MAELPPGRLATTEDYFAQQAKQAVTPDVMAQLAYMNYIDFISPFYSRGCSFEAWELKHTPQRVIKYSIAFYAYGLASVALIDPKLRALAGHDLDIAVSKMKCKRVWGDWEEDGFGTDPIEKENIMYKGHLNLMYGLYQLVTGSRRYEAEHAHLTRIIHDEIAANPFAGIVCEPDNYFVQCNSVAYLSLWVYDRLHGTDYRAATRAWLDFIQKDLIDPERGAFYLSYHPESGAVKPWISAYTTAWTLAMVHGMDPAFSERYYPRFKQTFVEVYDEGRKARVRETAGTDDADGGVGLASAFTLLLAREMGDQQLFDQLLNHLEPPAKPSIVSASLRYEHPGSVLFDELLFLAKVHAGFGALLRMPPPAAKLAGKHHHHHH

SEQ ID NO: 37 - protein, 372 aa, Artificial Sequence
  Description of Artificial Sequence Synthetic polypeptide
MAELPPGRLATTEDYFAQQAKQAVTPDVMAQLAYMNYIDFISPFYSRGCSFEAWELKHTPQRVIKYSIAFYAYGLASVALIDPKLRALAGHDLDIAVSKMKCKRVWGDWEEDGFGTDPIEKENIMYKGHLNLMYGLYQLVTGSRRYEAEHAHLTRIIHDEIAANPFAGIVCEPDNYFVQCNSVAYLSLWVYDRLHGTDYRAATRAWLDFIQKDLIDPERGAFYLSYHPESGAVKPWISAYTTAWTLAMVHGMDPAFSERYYPRFKQTFVEVYDEGRKARVRETAGTDDADGGVGLASAFTLLLAREMGDQQLFDQLLNHLEPPAKPSIVSASLRYEHPGSLLFDELLFLAKVHAGFGALLRMPPPAAKLAGK

SEQ ID NO: 38 - protein, 403 aa, Artificial Sequence
  Description of Artificial Sequence Synthetic polypeptide
MRFTLKTTAIVSAAALLAGFGPPPRAAELPPGRLATTEDYFAQQAKQAVTPDVMAQLAYMNYIDFISPFYSRGCSFEAWELKHTPQRVIKYSIAFYAYGLASVALIDPKLRALAGHDLDIAVSKMKCKRVWGDWEEDGFGTDPIEKENIMYKGHLNLMYGLYQLVTGSRRYEAEHAHLTRIIHDEIAANPFAGIVCEPDNYFVQCNSVAYLSLWVYDRLHGTDYRAATRAWLDFIQKDLIDPERGAFYLSYHPESGAVKPWISAYTTAWTLAMVHGMDPAFSERYYPRFKQTFVEVYDEGRKARVRETAGTDDADGGVGLASAFTLLLAREMGDQQLFDQLLNHLEPPAKPSIVSASLRYEHPGSLLFDELLFLAKVHAGFGALLRMPPPAAKLAGKHHHHHH

SEQ ID NO: 39 - protein, 6 aa, Artificial Sequence
  Description of Artificial Sequence Synthetic 6xHis tag
HHHHHH

SEQ ID NO: 40 - protein, 372 aa, Artificial Sequence
  Description of Artificial Sequence Synthetic polypeptide
MAELPPGRLATTEDYFAQQAKQAVTPDVMAQLAYMNYIDFISPFYSRGCSFEAWELKHTPQRVIKYSIAFYAYGLASVALIDPKLRALAGHDLDIAVSKMKCKRVWGDWEEDGFGTDPIEKENIMYKGHLNLMYGLYQLVTGSRRYEAEHAHLTRIIHDEIAANPFAGIVCEPDNYFVQCNSVAYLSLWVYDRLHGTDYRAATREWLDFIQKDLIDPERGAFYLSYHPESGAVKPWISAYTTAWTLAMVHGMDPAFSERYYPRFKQTFVEVYDEGRKARVRETAGTDDADGGVGLASAFTLLLAREMGDQQLFDQLLNHLEPPAKPSIVSASLRYEHPGSLLFDELLFLAKVHAGFGALLRMPPPAAKLAGK

SEQ ID NO: 41 - protein, 397 aa, Artificial Sequence
  Description of Artificial Sequence Synthetic polypeptide
MRFTLKTTAIVSAAALLAGFGPPPRAAELPPGRLATTEDYFAQQAKQAVTPDVMAQLAYMNYIDFISPFYSRGCSFEAWELKHTPQRVIKYSIAFYAYGLASVALIDPKLRALAGHDLDIAVSKMKCKRVWGDWEEDGFGTDPIEKENIMYKGHLNLMYGLYQLVTGSRRYEAEHAHLTRIIHDEIAANPFAGIVCEPDNYFVQCNSVAYLSLWVYDRLHGTDYRAATREWLDFIQKDLIDPERGAFYLSYHPESGAVKPWISAYTTAWTLAMVHGMDPAFSERYYPRFKQTFVEVYDEGRKARVRETAGTDDADGGVGLASAFTLLLAREMGDQQLFDQLLNHLEPPAKPSIVSASLRYEHPGSLLFDELLFLAKVHAGFGALLRMPPPAAKLAGK

SEQ ID NO: 42 - protein, 371 aa, Artificial Sequence
  Description of Artificial Sequence Synthetic polypeptide
AELPPGRLATTEDYFAQQAKQAVTPDVMAQLAYMNYIDFISPFYSRGCSFEAWELKHTPQRVIKYSIAFYAYGLASVALIDPKLRALAGHDLDIAVSKMKCKRVWGDWEEDGFGTDPIEKENIMYKGHLNLMYGLYQLVTGSRRYEAEHAHLTRIIHDEIAANPFAGIVCEPDNYFVQCNSVAYLSLWVYDRLHGTDYRAATREWLDFIQKDLIDPERGAFYLSYHPESGAVKPWISAYTTAWTLAMVHGMDPAFSERYYPRFKQTFVEVYDEGRKARVRETAGTDDADGGVGLASAFTLLLAREMGDQQLFDQLLNHLEPPAKPSIVSASLRYEHPGSLLFDELLFLAKVHAGFGALLRMPPPAAKLAGK

SEQ ID NO: 43 - protein, 372 aa, Artificial Sequence
  Description of Artificial Sequence Synthetic polypeptide
MAELPPGRLATTEDYFAQQAKQAVTPDVMAQLAYMNYIDFISPFYSRGCSFEAWELKHTPQRVIKYSIAFYAYGLASVALIDPKLRALAGHDLDIAVSKMKCKRVWGDWEEDGFGTDPIEKENIMYKGHLNLMYGLYQLVTGSRRYEAEHAHLTRIIHDEIAANPFAGIVCEPDNYFVQCNSVAYLSLWVYDRLHGTDYRAATRAWLDFIQKDLIDPERGAFYLSYHPESGAVKPWISAYTTAWTLAMVHGMDPAFSERYYPRFKQTFVEVYDEGRKARVRETAGTDDADGGVGLASAFTLLLAREMGDQQLFDQLLNHLEPPAKPSIVSASLRYEHPGSVLFDELLFLAKVHAGFGALLRMPPPAAKLAGK

SEQ ID NO: 44 - protein, 397 aa, Artificial Sequence
  Description of Artificial Sequence Synthetic polypeptide
MRFTLKTTAIVSAAALLAGFGPPPRAAELPPGRLATTEDYFAQQAKQAVTPDVMAQLAYMNYIDFISPFYSRGCSFEAWELKHTPQRVIKYSIAFYAYGLASVALIDPKLRALAGHDLDIAVSKMKCKRVWGDWEEDGFGTDPIEKENIMYKGHLNLMYGLYQLVTGSRRYEAEHAHLTRIIHDEIAANPFAGIVCEPDNYFVQCNSVAYLSLWVYDRLHGTDYRAATRAWLDFIQKDLIDPERGAFYLSYHPESGAVKPWISAYTTAWTLAMVHGMDPAFSERYYPRFKQTFVEVYDEGRKARVRETAGTDDADGGVGLASAFTLLLAREMGDQQLFDQLLNHLEPPAKPSIVSASLRYEHPGSVLFDELLFLAKVHAGFGALLRMPPPAAKLAGK

SEQ ID NO: 45 - protein, 371 aa, Artificial Sequence
  Description of Artificial Sequence Synthetic polypeptide
AELPPGRLATTEDYFAQQAKQAVTPDVMAQLAYMNYIDFISPFYSRGCSFEAWELKHTPQRVIKYSIAFYAYGLASVALIDPKLRALAGHDLDIAVSKMKCKRVWGDWEEDGFGTDPIEKENIMYKGHLNLMYGLYQLVTGSRRYEAEHAHLTRIIHDEIAANPFAGIVCEPDNYFVQCNSVAYLSLWVYDRLHGTDYRAATRAWLDFIQKDLIDPERGAFYLSYHPESGAVKPWISAYTTAWTLAMVHGMDPAFSERYYPRFKQTFVEVYDEGRKARVRETAGTDDADGGVGLASAFTLLLAREMGDQQLFDQLLNHLEPPAKPSIVSASLRYEHPGSVLFDELLFLAKVHAGFGALLRMPPPAAKLAGK

SEQ ID NO: 46 - protein, 372 aa, Artificial Sequence
  Description of Artificial Sequence Synthetic polypeptide
MAELPPGRLATTEDYFAQQAKQAVTPDVMAQLAYMNYIDFISPFYSRGCSFEAWELKHTPQRVIKYSIAFYAYGLASVALIDPKLRALAGHDLDIAVSKMKCKRVWGDWEEDGFGTDPIEKENIMYKGHLNLMYGLYQLVTGDRRYEAEHAHLTRIIHDEIAANPFAGIVCEPDNYFVQCNSVAYLSLWVYDRLHGTDYRAATRAWLDFIQKDLIDPERGAFYLSYHPESGAVKPWISAYTTAWTLAMVHGMDPAFSERYYPRFKQTFVEVYDEGRKARVRETAGTDDADGGVGLASAFTLLLAREMGDQQLFDQLLNHLEPPAKPSIVSASLRYEHPGSLLFDELLFLAKVHAGFGALLRMPPPAAKLAGK

SEQ ID NO: 47 - protein, 397 aa, Artificial Sequence
  Description of Artificial Sequence Synthetic polypeptide
MRFTLKTTAIVSAAALLAGFGPPPRAAELPPGRLATTEDYFAQQAKQAVTPDVMAQLAYMNYIDFISPFYSRGCSFEAWELKHTPQRVIKYSIAFYAYGLASVALIDPKLRALAGHDLDIAVSKMKCKRVWGDWEEDGFGTDPIEKENIMYKGHLNLMYGLYQLVTGDRRYEAEHAHLTRIIHDEIAANPFAGIVCEPDNYFVQCNSVAYLSLWVYDRLHGTDYRAATRAWLDFIQKDLIDPERGAFYLSYHPESGAVKPWISAYTTAWTLAMVHGMDPAFSERYYPRFKQTFVEVYDEGRKARVRETAGTDDADGGVGLASAFTLLLAREMGDQQLFDQLLNHLEPPAKPSIVSASLRYEHPGSLLFDELLFLAKVHAGFGALLRMPPPAAKLAGK

SEQ ID NO: 48 - protein, 371 aa, Artificial Sequence
  Description of Artificial Sequence Synthetic polypeptide
AELPPGRLATTEDYFAQQAKQAVTPDVMAQLAYMNYIDFISPFYSRGCSFEAWELKHTPQRVIKYSIAFYAYGLASVALIDPKLRALAGHDLDIAVSKMKCKRVWGDWEEDGFGTDPIEKENIMYKGHLNLMYGLYQLVTGDRRYEAEHAHLTRIIHDEIAANPFAGIVCEPDNYFVQCNSVAYLSLWVYDRLHGTDYRAATRAWLDFIQKDLIDPERGAFYLSYHPESGAVKPWISAYTTAWTLAMVHGMDPAFSERYYPRFKQTFVEVYDEGRKARVRETAGTDDADGGVGLASAFTLLLAREMGDQQLFDQLLNHLEPPAKPSIVSASLRYEHPGSLLFDELLFLAKVHAGFGALLRMPPPAAKLAGK

SEQ ID NO: 49 - protein, 25 aa, Artificial Sequence
  Description of Artificial Sequence Synthetic peptide
RFTLKTTAIVSAAALLAGFGPPPRA

SEQ ID NO: 50 - protein, 26 aa, Artificial Sequence
  Description of Artificial Sequence Synthetic peptide
MRFTLKTTAIVSAAALLAGFGPPPRA

SEQ ID NO: 51 - protein, 371 aa, Castellaniella defragrans
AELPPGRLATTEDYFAQQAKQAVTPDVMAQLAYMNYIDFISPFYSRGCSFEAWELKHTPQRVIKYSIAFYAYGLASVALIDPKLRALAGHDLDIAVSKMKCKRVWGDWEEDGFGTDPIEKENIMYKGHLNLMYGLYQLVTGSRRYEAEHAHLTRIIHDEIAANPFAGIVCEPDNYFVQCNSVAYLSLWVYDRLHGTDYRAATRAWLDFIQKDLIDPERGAFYLSYHPESGAVKPWISAYTTAWTLAMVHGMDPAFSERYYPRFKQTFVEVYDEGRKARVRETAGTDDADGGVGLASAFTLLLAREMGDQQLFDQLLNHLEPPAKPSIVSASLRYEHPGSLLFDELLFLAKVHAGFGALLRMPPPAAKLAGK

(19) United States
(12) Patent Application Publication
(10) Pub. No.: US 2016/0251644 A1
(43) Pub. Date: Sep. 1, 2016

(54) NOVEL POLYPEPTIDES AND USES THEREOF

(71) Applicant: INVISTA NORTH AMERICA S.A.R.L., WILMINGTON, DE (US)

(72) Inventors: ADRIANA L. BOTES, Rosedale East (GB); Nadia Kadi, Marton (GB)

(73) Assignee: INVISTA NORTH AMERICA S.A.R.L., WILMINGTON, DE (US)

(21) Appl. No.: 15/055,493

(22) Filed: Feb. 26, 2016

Related U.S. Application Data
(60) Provisional application No. 62/126,315, filed on Feb. 27, 2015.

(51) Int. Cl. C12N 9/88; C11B 9/00; C08F 136/06; C07K 16/40; C12P 5/02; C08F 136/08; C07C 11/21; C07C 11/18; C07C 11/167; A61K 31/01; C08F 136/22

(52) U.S. Cl. CPC C12N 9/88; A61K 31/01; C11B 9/0007; C08F 136/06; C07K 16/40; C08F 136/22; C08F 136/08; C12Y 402/01127; C07C 11/21; C07C 11/18; C07C 11/167; C12P 5/026

(57) ABSTRACT

The present disclosure provides novel polypeptides with improved 3-buten-2-ol dehydratase activity, polypeptides with improved linalool dehydratase activity, and polypeptides with catalytic activity in the conversion of 3-methyl-3-buten-2-ol to isoprene. Methods of making and using the polypeptides are also provided.

### SEQUENCE LISTING

The instant application contains a Sequence Listing which has been submitted electronically in ASCII format and is hereby incorporated by reference in its entirety. Said ASCII copy, created on Feb. 26, 2016, is named 12444.0299-00304_SL.txt and is 149,887 bytes in size.

### FIELD

The present disclosure provides novel polypeptides with improved 3-buten-2-ol dehydratase activity, polypeptides with improved linalool dehydratase activity, and polypeptides with catalytic activity in the conversion of 3-methyl-3-buten-2-ol to isoprene. Methods of making and using the polypeptides are also provided.

### BACKGROUND

Linalool dehydratase (EC 4.2.1.127) is a unique bi-functional enzyme which naturally catalyzes the dehydration of linalool to myrcene and the isomerization of linalool to geraniol. LDH can also catalyze the conversion of 3-methyl-3-buten-2-ol into isoprene. See PCT/US2013/045430, published as WO/2013/188546 and US Patent Publication No. 20150037860 herein incorporated by reference in their entireties. Isoprene can also be synthesized by other methods. See US Patent Publication Nos. 20150037860 and 20130217081, herein incorporated by reference in their entireties.

1,3-Butadiene (hereinafter butadiene) is an important monomer for the production of synthetic rubbers including styrene-butadiene-rubber (SBR), polybutadiene (PB), styrene-butadiene latex (SBL), acrylonitrile-butadiene-styrene resins (ABS), nitrile rubber, and adiponitrile, which is used in the manufacture of Nylon-66 (White, Chemico-Biological Interactions, 2007, 166, 10-14).

Butadiene is typically produced as a co-product from the steam cracking process, distilled to a crude butadiene stream, and purified via extractive distillation (White, Chemico-Biological Interactions, 2007, 166, 10-14). Industrially, 95% of global butadiene production is undertaken via the steam cracking process using petrochemical-based feedstocks such as naphtha. Butadiene has also been prepared, among other methods, by dehydrogenation of n-butane and n-butene (Houdry process) and oxidative dehydrogenation of n-butene (Oxo-D or O-X-D process) (White, Chemico-Biological Interactions, 2007, 166, 10-14). These methods are associated with high cost of production and low process yield (White, Chemico-Biological Interactions, 2007, 166, 10-14). Isoprene is an important monomer for the production of specialty elastomers including motor mounts/fittings, surgical gloves, rubber bands, golf balls and shoes. Styrene-isoprene-styrene block copolymers form a key component of hot-melt pressure-sensitive adhesive formulations and cis-poly-isoprene is utilised in the manufacture of tires (Whited et al., Industrial Biotechnology, 2010, 6(3), 152-163).

Manufacturers of rubber goods depend on either imported natural rubber from the Brazilian rubber tree or petroleum-based synthetic rubber polymers (Whited et al., 2010, supra). Given a reliance on petrochemical feedstocks and energy intensive catalytic steps, biotechnology offers an alternative approach to butadiene synthesis via biocatalysis. Biocatalysis is the use of biological catalysts, such as enzymes, to perform biochemical transformations of organic compounds. Accordingly, there is a need for sustainable methods for producing butadiene, wherein the methods are biocatalyst-based (Jang et al, Biotechnology & Bioengineering, 2012, 109(10), 2437-2459). Both bioderived feedstocks and petrochemical feedstocks are viable starting materials for the biocatalysis processes.

### SUMMARY

This disclosure provides novel, recombinant, polypeptides that can catalyze the dehydration of 3-buten-2-ol to 1,3-butadiene, the dehydration of linalool to myrcene, and that of 3-methyl-3-buten-2-ol into isoprene. These novel polypeptides and their reaction products have numerous industrial applications including, but not limited to, uses in polymer biosynthesis, pharmacology (analgesics, anti-inflammatories, sedatives, etc. comprising myrcene), and in the perfume industry (e.g., myrcene as a component of fragrances).

LDH is known to catalyze the dehydration of linalool to myrcene and the isomerization of linalool to geraniol. It has now been discovered that LDH from Castellaniella defragrans is also able to convert 3-buten-2-ol to 1,3-butadiene, albeit in low yields. Provided herein are novel polypeptides with advantageous properties in industrial synthesis of 1,3-butadiene. These polypeptides exhibit improved linalool dehydratase activity, and/or 3-buten-2-ol dehydratase activity, relative to that of wild-type LDH, have linalool isomerase activity, and also have improved linalool dehydratase activity leading to myrcene formation as well as improved activity in the catalysis of the conversion of 3-methyl-3-buten-2-ol into isoprene.

Also provided herein are novel polypeptides with increased solubility, relatively to that of wild-type LDH of Castellaniella defragrans.

One embodiment provides a polypeptide comprising an amino acid sequence with at least 90% amino acid sequence homology to SEQ ID NO:11, wherein said amino acid sequence comprises at least 1-3 alteration(s) relative to SEQ ID NO:11 independently selected from:
a substitution of the amino acid that occupies position 58 with a different amino acid selected from R and equivalent amino acids;
a substitution of the amino acid that occupies position 83 with a different amino acid selected from A and equivalent amino acids; and
a substitution of the amino acid that occupies position 252 with a different amino acid selected from A and equivalent amino acids.

One embodiment provides said polypeptide of paragraph [009], wherein said amino acid sequence has at least 91% amino acid sequence homology to SEQ ID NO:11, preferably at least 92% amino acid sequence homology to SEQ ID NO:11, preferably at least 93% amino acid sequence homology to SEQ ID NO:11, preferably at least 94% amino acid sequence homology to SEQ ID NO:11, preferably at least 95% amino acid sequence homology to SEQ ID NO:11, preferably at least 96% amino acid sequence homology to SEQ ID NO:11, preferably at least 97% amino acid sequence homology to SEQ ID NO:11, preferably at least 98% amino acid sequence homology to SEQ ID NO:11, or preferably at least 99% amino acid sequence homology to SEQ ID NO:11.

In one further embodiment, said polypeptide of any one of paragraphs [009] and [010] is such that said amino acid sequence comprises one of the following alterations relative to SEQ ID NO. 11:
a substitution of the amino acid that occupies position 58 with a different amino acid selected from R and equivalent amino acids;
a substitution of the amino acid that occupies position 83 with a different amino acid selected from A and equivalent amino acids; and
a substitution of the amino acid that occupies position 252 with a different amino acid selected from A and equivalent amino acids.

In one further embodiment, said polypeptide of paragraphs [009] and [010] is such that said amino acid sequence comprises two of the following alterations relative to SEQ ID NO. 11:
a substitution of the amino acid that occupies position 58 with a different amino acid selected from R and equivalent amino acids;
a substitution of the amino acid that occupies position 83 with a different amino acid selected from A and equivalent amino acids; and
a substitution of the amino acid that occupies position 252 with a different amino acid selected from A and equivalent amino acids.

In one further embodiment, said polypeptide of paragraphs [009] and [010] is such that said amino acid sequence comprises three of the following alterations relative to SEQ ID NO. 11:
a substitution of the amino acid that occupies position 58 with a different amino acid selected from R and equivalent amino acids;
a substitution of the amino acid that occupies position 83 with a different amino acid selected from A and equivalent amino acids; and
a substitution of the amino acid that occupies position 252 with a different amino acid selected from A and equivalent amino acids.

In one further embodiment, said polypeptide of paragraphs [009] and [010] is such that said amino acid sequence comprises a substitution of the amino acid that occupies position 58 with a different amino acid selected from R and equivalent amino acids.

In one further embodiment, said polypeptide of paragraphs [009] and [010] is such that said amino acid sequence comprises a substitution of the amino acid that occupies position 83 with a different amino acid selected from A and equivalent amino acids.

In one further embodiment, said polypeptide of paragraphs [009] and [010] is such that said amino acid sequence comprises a substitution of the amino acid that occupies position 252 with a different amino acid selected from A and equivalent amino acids.

In one further embodiment, said polypeptide of paragraphs [009] and [010] is such that said amino acid sequence comprises a A58R substitution.

In one further embodiment, said polypeptide of paragraphs [009] and [010] is such that said amino acid sequence comprises a H83A substitution.

In one further embodiment, said polypeptide of paragraphs [009] and [010] is such that said amino acid sequence comprises a H252A substitution.

In one further embodiment, said polypeptide of paragraphs [009] and [010] is such that said amino acid sequence comprises a A58R substitution and a H83A substitution.

In one further embodiment, said polypeptide of paragraphs [009] and [010] is such that said amino acid sequence comprises a A58R substitution and a H252A substitution.

In one further embodiment, said polypeptide of paragraphs [009] and [010] is such that said amino acid sequence comprises a H83A substitution and a H252A substitution.

In one further embodiment, said polypeptide of paragraphs [009] and [010] is such that said amino acid sequence comprises a A58R substitution, a H83A substitution, and a H252A substitution.

In one further embodiment, said polypeptide of paragraphs [009] and [010] is such that said amino acid sequence is SEQ ID NO. 25, or SEQ ID NO. 25 without the C-terminal His-Tag.

In one further embodiment, said polypeptide of paragraphs [009] and [010] is such that said amino acid sequence is SEQ ID NO. 14, or SEQ ID NO. 14 without the C-terminal His-Tag.

In one further embodiment, said polypeptide of paragraphs [009] and [010] is such that said amino acid sequence is SEQ ID NO. 15, or SEQ ID NO. 15 without the C-terminal His-Tag.

In another embodiment, said polypeptide of any one of paragraphs [009] to [026] is such that polypeptide has a solubility that is increased about 1.5 fold or greater when compared to that of a polypeptide consisting of SEQ ID NO: 11, 13, 37, OR 38, preferably about 2 fold or greater when compared to that of a polypeptide consisting of SEQ ID NO: 11, 13, 37, OR 38, preferably about 2.5 or greater when compared to that of a polypeptide consisting of SEQ ID NO: 11, 13, 37, OR 38, preferably about 3 fold or greater when compared to that of a polypeptide consisting of SEQ ID NO: 11, 13, 37, OR 38, when compared to that of a polypeptide consisting of SEQ ID NO: 11, 13, 37, OR 38, preferably about 4 fold or greater when compared to that of a polypeptide consisting of SEQ ID NO: 11, 13, 37, OR 38, preferably about 4.5 fold or greater when compared to that of a polypeptide consisting of SEQ ID NO: 11, 13, 37, OR 38, or preferably about 5 fold or greater when compared to that of a polypeptide consisting of SEQ ID NO: 11, 13, 37, OR 38, and wherein the increased solubility is observed in at least one solubility assay.

In another embodiment of the polypeptide according to paragraph [027], the increased solubility is observed in at least one type of non-bacterial cells.

In another embodiment of the polypeptide according to paragraph [027], the increased solubility is observed in at least one type of bacteria.

In another embodiment of the polypeptide according to paragraph [027], the increased solubility is observed in more than one type of bacteria.

In another embodiment of the polypeptide according to paragraphs [029]-[030], the bacteria are a strain of E. coli.

In another embodiment of the polypeptide according to paragraph [031], the bacteria are Origami2(DE3), BL21(DE3), or a related strain.

Another embodiment provides a polypeptide comprising an amino acid sequence with at least 90% amino acid sequence homology to SEQ ID NO:11, wherein said amino acid sequence comprises 1-3 alteration(s) relative to SEQ ID NO:11 independently selected from:
a substitution of the amino acid that occupies position 168 with a different amino acid selected from D and equivalent amino acids;
a substitution of the amino acid that occupies position 230 with a different amino acid selected from E and equivalent amino acids; and
a substitution of the amino acid that occupies position 366 with a different amino acid selected from V and equivalent amino acids.

One embodiment provides said polypeptide of paragraph [033], wherein said amino acid sequence has at least 91% amino acid sequence homology to SEQ ID NO:11, preferably at least 92% amino acid sequence homology to SEQ ID NO:11, preferably at least 93% amino acid sequence homology to SEQ ID NO:11, preferably at least 94% amino acid sequence homology to SEQ ID NO:11, preferably at least 95% amino acid sequence homology to SEQ ID NO:11, preferably at least 96% amino acid sequence homology to SEQ ID NO:11, preferably at least 97% amino acid sequence homology to SEQ ID NO:11, preferably at least 98% amino acid sequence homology to SEQ ID NO:11, or preferably at least 99% amino acid sequence homology to SEQ ID NO:11.

Another embodiment provides the polypeptide according to any one of paragraphs [033] and [034], wherein said amino acid sequence comprises one of the following alterations relative to SEQ ID NO. 11:
a substitution of the amino acid that occupies position 168 with a different amino acid selected from D and equivalent amino acids;
a substitution of the amino acid that occupies position 230 with a different amino acid selected from E and equivalent amino acids; and
a substitution of the amino acid that occupies position 366 with a different amino acid selected from V and equivalent amino acids.

Another embodiment provides the polypeptide according to any one of paragraphs [033] and [034], wherein said amino acid sequence comprises two of the following alterations relative to SEQ ID NO. 11:
a substitution of the amino acid that occupies position 168 with a different amino acid selected from D and equivalent amino acids;
a substitution of the amino acid that occupies position 230 with a different amino acid selected from E and equivalent amino acids; and
a substitution of the amino acid that occupies position 366 with a different amino acid selected from V and equivalent amino acids.

Another embodiment provides the polypeptide according to any one of paragraphs [033] and [034], wherein said amino acid sequence comprises three of the following alterations relative to SEQ ID NO. 11:
a substitution of the amino acid that occupies position 168 with a different amino acid selected from D and equivalent amino acids;
a substitution of the amino acid that occupies position 230 with a different amino acid selected from E and equivalent amino acids; and
a substitution of the amino acid that occupies position 366 with a different amino acid selected from V and equivalent amino acids.

Another embodiment provides the polypeptide according to any one of paragraphs [033] and [034], wherein said amino acid sequence comprises a substitution of the amino acid that occupies position 168 with a different amino acid selected from D and equivalent amino acids.

Another embodiment provides the polypeptide according to any one of paragraphs [033] and [034], wherein said amino acid sequence comprises a substitution of the amino acid that occupies position 230 with a different amino acid selected from E and equivalent amino acids.

Another embodiment provides the polypeptide according to any one of paragraphs [033] and [034], wherein said amino acid sequence comprises a substitution of the amino acid that occupies position 367 with a different amino acid selected from V and equivalent amino acids.

Another embodiment provides the polypeptide according to any one of paragraphs [033] and [034], wherein said amino acid sequence comprises a S168D substitution.

Another embodiment provides the polypeptide according to any one of paragraphs [033] and [034], wherein said amino acid sequence comprises a A230E substitution.

The polypeptide according to any one of claims 25 and 26, wherein said amino acid sequence comprises a L366V substitution.

Another embodiment provides the polypeptide according to any one of paragraphs [033] and [034], wherein said amino acid sequence comprises a S168D substitution and a A230E substitution.

Another embodiment provides the polypeptide according to any one of paragraphs [033] and [034], wherein said amino acid sequence comprises a S168D substitution and a L366V substitution.

Another embodiment provides the polypeptide according to any one of paragraphs [033] and [034], wherein said amino acid sequence comprises a A230E substitution and a L366V substitution.

Another embodiment provides the polypeptide according to any one of paragraphs [033] and [034], wherein said amino acid sequence comprises a S168D substitution, a A230E substitution, and a L366V substitution.

Another embodiment provides the polypeptide according to any one of paragraphs [033] and [034], wherein said amino acid sequence is SEQ ID NO. 32, or SEQ ID NO. 32 without the C-terminal His-Tag.

Another embodiment provides the polypeptide according to any one of paragraphs [033] and [034], wherein said amino acid sequence is SEQ ID NO. 35, or SEQ ID NO. 35 without the C-terminal His-Tag.

Another embodiment provides the polypeptide according to any one of paragraphs [033] and [034], wherein said amino acid sequence is SEQ ID NO. 36, or SEQ ID NO. 36 without the C-terminal His-Tag.

Another embodiment provides the polypeptide according to any one of paragraphs [033] to [050], wherein the polypeptide has a specific activity in the catalysis of the dehydration of 3-buten-2-ol to 1,3-butadiene and/or in the catalysis of the dehydration of 3-methyl-3-buten-2-ol to isoprene when compared to that of a polypeptide consisting of SEQ ID NO: 11, 13, 37, or 38 that is increased about 1.5 fold or greater, preferably about 2 fold or greater when compared to that of a polypeptide consisting of SEQ ID NO: 11, 13, 37, OR 38, preferably about 2.5 fold or greater when compared to that of a polypeptide consisting of SEQ ID NO: 11, 13, 37, OR 38, preferably about 3 fold or greater when compared to that of a polypeptide consisting of SEQ ID NO: 11, 13, 37, OR 38, preferably about 3.5 fold or greater when compared to the parent LDH to that of a polypeptide consisting of SEQ ID NO: 11, 13, 37, OR 38, preferably about 4 fold or greater when compared to that of a polypeptide consisting of SEQ ID NO: 11, 13, 37, OR 38, preferably about 4.5 fold or greater when compared to that of a polypeptide consisting of SEQ ID NO: 11, 13, 37, OR 38, or preferably about 5 fold or greater when compared to that of a polypeptide consisting of SEQ ID NO: 11, 13, 37, OR 38, or preferably about 15 fold or greater when compared to that of a polypeptide consisting of SEQ ID NO: 11, 13, 37, OR 38, or preferably about 30 fold or greater when compared to that of a polypeptide consisting of SEQ ID NO: 11, 13, 37, or 38 or preferably about 55 fold or greater when compared to that of a polypeptide consisting of SEQ ID NO: 11, 13, 37, OR 38, and wherein the increased specific activity is observed in at least one specific activity assay.

Another embodiment provides the polypeptide according to any one of paragraphs [033] to [051], wherein the increased specific activity in the catalysis of the dehydration of 3-buten-2-ol to 1,3-butadiene and/or in the catalysis of the dehydration of 3-methyl-3-buten-2-ol to isoprene is observed in at least one type of non-bacterial cells.

Another embodiment provides the polypeptide according to any one of paragraphs [033] to [051], wherein the increased specific activity in the catalysis of the dehydration of 3-buten-2-ol to 1,3-butadiene and/or in the catalysis of the dehydration of 3-methyl-3-buten-2-ol to isoprene is observed in at least one type of bacteria.

Another embodiment provides the polypeptide according to any one of paragraphs [033] to [051], wherein the increased specific activity in the catalysis of the dehydration of 3-buten-2-ol to 1,3-butadiene and/or in the catalysis of the dehydration of 3-methyl-3-buten-2-ol to isoprene is observed in more than one type of bacteria.

Another embodiment provides the polypeptide according to paragraph [054], wherein the bacteria are a strain of E. Coli.

Another embodiment provides the polypeptide according to any one of paragraphs [053] to [055], wherein the bacteria are Origami2(DE3), BL21(DE3), or a related strain.

Another further embodiment provides the polypeptide according to any one of paragraphs [009] to [032], wherein said amino acid sequence further comprises an additional 1-3 alteration(s) relative to SEQ ID NO:11 independently selected from:
a substitution of the amino acid that occupies position 168 with a different amino acid selected from D and equivalent amino acids;
a substitution of the amino acid that occupies position 230 with a different amino acid selected from E and equivalent amino acids; and
a substitution of the amino acid that occupies position 366 with a different amino acid selected from V and equivalent amino acids.

Another further embodiment provides the polypeptide according to any one of paragraphs [009] to [032], wherein said polypeptide has a specific activity in the catalysis of the dehydration of 3-buten-2-ol to 1,3-butadiene and/or in the catalysis of the dehydration of 3-methyl-3-buten-2-ol to isoprene that is increased about 1.5 fold or greater when compared to that of a polypeptide consisting of SEQ ID NO: 11, 13, 37, OR 38, preferably about 2 fold or greater when compared to that of a polypeptide consisting of SEQ ID NO:13, preferably about 2.5 fold or greater when compared to that of a polypeptide consisting of SEQ ID NO: 11, 13, 37, OR 38, preferably about 3 fold or greater when compared to that of a polypeptide consisting of SEQ ID NO: 11, 13, 37, OR 38, preferably about 3.5 fold or greater when compared to the parent LDH to that of a polypeptide consisting of SEQ ID NO: 11, 13, 37, OR 38, preferably about 4 fold or greater when compared to that of a polypeptide consisting of SEQ ID NO: 11, 13, 37, OR 38, preferably about 4.5 fold or greater when compared to that of a polypeptide consisting of SEQ ID NO: 11, 13, 37, OR 38, or preferably about 5 fold or greater when compared to that of a polypeptide consisting of SEQ ID NO: 11, 13, 37, OR 38, or preferably about 15 fold or greater when compared to that of a polypeptide consisting of SEQ ID NO: 11, 13, 37, OR 38, or preferably about 30 fold or greater when compared to that of a polypeptide consisting of SEQ ID NO: 11, 13, 37, or 38 or preferably about 55 fold or greater when compared to that of a polypeptide consisting of SEQ ID NO: 11, 13, 37, OR 38, and wherein the increased specific activity is observed in at least one specific activity assay.

Another further embodiment provides the polypeptide according to any one of paragraphs [009] to [058], wherein the polypeptide has both alterations that improve solubility and alterations that improve specific activity in the catalysis of the dehydration of 3-buten-2-ol to 1,3-butadiene. In a further embodiment, the polypeptide also has improved activity in the catalysis of the dehydration of 3-methyl-3-buten-2-ol to isoprene.

Another further embodiment provides the polypeptide according to any one of paragraphs [009] to [058], wherein one or more additional substitutions, deletions, insertions, and/or inversions are introduced into the polypeptide.

Another further embodiment provides the polypeptide according to any one of paragraphs [009] to [059], wherein the polypeptide further contains an N-terminal periplasmic tag.

Another further embodiment provides the polypeptide according to any one of paragraphs [009] to [059], wherein the polypeptide lacks an N-terminal periplasmic tag.

Another further embodiment provides the polypeptide according to any one of paragraphs [009] to [059], wherein the polypeptide further contains an N-terminal periplasmic tag and a C-terminal poly-His tag.

Another further embodiment provides the polypeptide according to any one of paragraphs [009] to [059], wherein the polypeptide lacks a N-terminal periplasmic tag and contains a C-terminal poly-His tag.

Another further embodiment provides the polypeptide according to any one of paragraphs [009] to [059], wherein the polypeptide further contains a C-terminal poly-His tag.

Another further embodiment provides the polypeptide according to any one of paragraphs [009] to [059], wherein the polypeptide lacks a C-terminal poly-His tag.

Another embodiment provides the polypeptide according to any one of paragraphs [009] to [032], wherein the amino acid sequence of the polypeptide is that of SEQ ID NO. 14 plus an N-terminal periplasmic tag.

Another embodiment provides the polypeptide according to any one of paragraphs [009] to [032], wherein the amino acid sequence of the polypeptide is that of SEQ ID NO. 15 plus an N-terminal periplasmic tag.

Another embodiment provides the polypeptide according to any one of paragraphs [009] to [032], wherein the amino acid sequence of the polypeptide is that of SEQ ID NO. 25 plus an N-terminal periplasmic tag.

Another further embodiment provides the polypeptide according to any one of paragraphs [033] to [056], wherein the amino acid sequence of the polypeptide is that of SEQ ID NO. 32 plus an N-terminal periplasmic tag.

Another further embodiment provides the polypeptide according to any one of paragraphs [033] to [056], wherein the amino acid sequence of the polypeptide is that of SEQ ID NO. 35 plus an N-terminal periplasmic tag.

Another further embodiment provides the polypeptide according to any one of paragraphs [033] to [056], wherein the amino acid sequence of the polypeptide is that of SEQ ID NO. 36 plus an N-terminal periplasmic tag.

Another further embodiment provides the polypeptide according to any one of paragraphs [009] to [032], wherein the amino acid sequence of the polypeptide is that of SEQ ID NO. 14 plus an N-terminal periplasmic tag and without the poly-His tag.

Another further embodiment provides the polypeptide according to any one of paragraphs [009] to [032], wherein the amino acid sequence of the polypeptide is that of SEQ ID NO. 15 plus an N-terminal periplasmic tag and without the poly-His tag.

Another further embodiment provides the polypeptide according to any one of paragraphs [009] to [032], wherein the amino acid sequence of the polypeptide is that of SEQ ID NO. 25 plus an N-terminal periplasmic tag and without the poly-His tag.

Another further embodiment provides the polypeptide according to any one of paragraphs [033] to [056], wherein the amino acid sequence of the polypeptide is that of SEQ ID NO. 32 plus an N-terminal periplasmic tag and without the poly-His tag.

Another further embodiment provides the polypeptide according to any one of paragraphs [033] to [056], wherein the amino acid sequence of the polypeptide is that of SEQ ID NO. 35 plus an N-terminal periplasmic tag and without the poly-His tag.

Another further embodiment provides the polypeptide according to any one of paragraphs [033] to [056], wherein the amino acid sequence of the polypeptide is that of SEQ ID NO. 36 plus an N-terminal periplasmic tag and without the poly-His tag.

In another embodiment, the disclosure provides a derivative of any one of the polypeptides according to any one of paragraphs [009] to [078].

In another embodiment, the disclosure provides a polynucleotide comprising or consisting essentially of a nucleic acid encoding any one of the polypeptides or derivatives according to any one of paragraphs [009] to [079], preferably codon-optimized.

In another embodiment, the disclosure provides the polynucleotide according to paragraph [080], wherein the polynucleotide is either a DNA molecule or an RNA molecule.

In another embodiment, the disclosure provides the DNA molecule of according to paragraph [081], further comprising a promoter operably linked to the nucleic acid sequence encoding a LDH polypeptide.

In another embodiment, the disclosure provides a recombinant expression vector comprising a DNA molecule according to any one of paragraphs [080] to [082].

In another embodiment, the disclosure provides a host cell which is transformed or transduced with a DNA molecule according to any one of paragraphs [080] to [082] or with a recombinant expression vector according to paragraph [083].

In another embodiment, the disclosure provides the cell of paragraph [084], wherein the DNA molecule or the recombinant expression vector is integrated into a chromosome of the cell.

In another embodiment, the disclosure provides a microorganism comprising a heterologous DNA molecule encoding a polypeptide according to any one of paragraphs [009] to [079].

In another embodiment, the disclosure provides a transgenic animal or plant comprising a heterologous DNA molecule encoding a polypeptide according to any one of paragraphs [009] to [079].

In another embodiment, the disclosure provides the microorganism of paragraph [086], wherein the microorganism is a bacterium or a fungus.

In another embodiment, the disclosure provides the microorganism of paragraph [088], wherein the microorganism is an E. coli bacterium or a Castellaniella defragrans bacterium.

In another embodiment, the disclosure provides a vector comprising a DNA molecule according to any one of paragraphs [080] to [082].

In another embodiment, the disclosure provides a method of producing a polypeptide according to any one of paragraphs [009] to [079], the method comprising: (i) preparing an expression construct which comprises a polynucleotide of paragraph [080], with a sequence encoding the polypeptide according to one of paragraphs [080] to [082] operably linked to one or more regulatory nucleotide sequences; (ii) transfecting or transforming a suitable host cell with the expression construct; (iii) expressing the recombinant polypeptide in said host cell; and (iv) isolating the recombinant polypeptide from said host cell or using the resultant host cell as is or as a cell extract.

In another embodiment, the disclosure provides a method of making a polypeptide with improved solubility and/or improved specific activity in the catalysis of the dehydration of 3-buten-2-ol to 1,3-butadiene and/or in the catalysis of the dehydration of 3-methyl-3-buten-2-ol to isoprene relative to that of a polypeptide consisting of SEQ ID NO: 11, 13, 37, OR 38, the method comprising preparing a polypeptide according to any one of paragraphs [009] to [079].

Another embodiment provides a composition comprising one or more polypeptides according to any one of paragraphs [009] to [079].

Another embodiment provides the composition of paragraph [093], further comprising the polypeptide of SEQ ID NO:11 with or without the N-terminal periplasmic tag.

Another embodiment provides the composition of paragraph [093], comprising one or more polypeptides according to any one of paragraphs [009] to [079] with improved solubility.

Another embodiment provides the composition of paragraph [093], comprising one or more polypeptides according to any one of paragraphs [009] to with improved specific activity in the catalysis of the dehydration of 3-buten-2-ol to 1,3-butadiene and/or in the catalysis of the dehydration of 3-methyl-3-buten-2-ol to isoprene.

Another embodiment provides the composition according to any one of paragraphs [093] to [096], further comprising 3-buten-2-ol. Another embodiment provides for a composition according to any one of paragraphs [093] to [096], (further) comprising 3-methyl-3-buten-2-ol.

Another embodiment provides the composition according to any one of paragraphs [093] to [097], further comprising 1,3-butadiene. Another embodiment provides for a composition according to any one of paragraphs [093] to [099], (further) comprising isoprene.

Another embodiment provides a composition comprising a rubber product polymerized from 1,3-butadiene produced in the presence of a polypeptide according to any one of paragraphs [009] to [079]. Another embodiment provides a composition comprising a rubber product polymerized from 3-methyl-3-buten-2-ol produced in the presence of a polypeptide according to any one of paragraphs [009] to [079].

Another embodiment provides a composition comprising a copolymer polymerized from 1,3-butadiene produced in the presence of a polypeptide according to any one of paragraphs [009] to [079]. Another embodiment provides a composition comprising a copolymer polymerized from 3-methyl-3-buten-2-ol produced in the presence of a polypeptide according to any one of paragraphs [009] to [079].

Another embodiment provides a composition comprising a plastic product polymerized from 1,3-butadiene produced in the presence of a polypeptide according to any one of paragraphs [009] to [079]. Another embodiment provides a composition comprising a plastic product polymerized from 3-methyl-3-buten-2-ol produced in the presence of a polypeptide according to any one of paragraphs [009] to [079].

Another embodiment provides an antibody capable of binding to a polypeptide according to any one of paragraphs [009] to [079].

Another embodiment provides a fusion protein comprising a polypeptide according to any one of claims paragraphs [009] to [079].

Another embodiment provides a complex comprising a polypeptide according to any one of paragraphs [009] to [079], said complex optionally further comprising 3-buten-2-ol.

Another embodiment provides a complex comprising a polypeptide according to any one of paragraphs [009] to [079], said complex optionally further comprising 3-methyl-3-buten-2-ol comprising a polypeptide according to any one of paragraphs [009] to [079].

Another embodiment provides a composition comprising 3-buten-2-ol and a means for producing 1,3-butadiene. Another embodiment provides a composition comprising 3-methyl-3-buten-2-ol and a means for producing isoprene.

Another embodiment provides a composition comprising a substrate and a means for enzymatically producing 1,3-butadiene from said substrate. Another embodiment provides a composition comprising a substrate and a means for enzymatically producing isoprene from said substrate.

Another embodiment provides a method of producing 1,3-butadiene comprising:

a step for enzymatically converting 3-buten-2-ol to 1,3-butadiene; and measuring and/or harvesting the 1,3-butadiene thereby produced. Another embodiment provides a method of producing isoprene comprising: a step for enzymatically converting 3-methyl-3-buten-2-ol to isoprene; and measuring and/or harvesting the isoprene thereby produced.

Another embodiment provides an apparatus comprising a container and a means for producing 1,3-butadiene. Another embodiment provides an apparatus comprising a container and a means for producing isoprene.

Another embodiment provides a method of designing a polypeptide with improved specific activity in the catalysis of the dehydration of 3-buten-2-ol to 1,3-butadiene relative to that of a polypeptide consisting of SEQ ID NO: 11, 13, 37, OR 38, the method comprising mutating a means for enzymatically converting 3-buten-2-ol to 1,3-butadiene. Another embodiment provides a method of designing a polypeptide with improved specific activity in the catalysis of the dehydration of 3-methyl-3-buten-2-ol to isoprene relative to that of a polypeptide consisting of SEQ ID NO: 11, 13, 37, OR 38, the method comprising mutating a means for enzymatically converting 3-methyl-3-buten-2-ol to isoprene.

Another embodiment provides a method of designing a polypeptide with improved solubility relative to that of a polypeptide consisting of SEQ ID NO: 11, 13, 37, OR 38, the method comprising mutating a means for enzymatically converting 3-buten-2-ol to 1,3-butadiene.

Another embodiment provides for polypeptide consisting of, or consisting essentially of, one of the peptides whose sequence is described in the SEQUENCE LISTING. In alternative set of embodiments and claims, % sequence homology is replaced with % sequence identity. In other words, “% amino acid sequence homology” can be, in alternate embodiments within the scope of this application, replaced by “% amino acid sequence identity.”

Other objects, features and advantages of the disclosed methods, systems and compositions will become apparent from the following detailed description. It should be understood, however, that the detailed description and the specific examples, while indicating specific embodiments, are given by way of illustration only, since various changes and modifications within the spirit and scope of the inventions provided herein will become apparent to those skilled in the art from this detailed description.

### BRIEF DESCRIPTION OF DRAWINGS

Those of skill in the art will understand that the drawings, described below, are for illustrative purposes only. The drawings are not intended to limit the scope of the present teachings in any way.

The patent or application file contains at least one drawing executed in color. Copies of this patent or patent application publication with color drawing(s) will be provided by the United States Patent and Trademark Office upon request and payment of the necessary fee.

FIG. 1: schematic overview of the reactions catalyzed by the linalool dehydratase from Castellaniella defragrans (SEQ ID NO:11; LDHcg1): a) the isomerization and dehydration of the natural substrate linalool; and b) the dehydration of 3-buten-2-ol to 1,3-butadiene.

FIG. 2: Test for butadiene formation of LDHcg1. Both constructs of the linalool dehydratase from C. defragrans (pPI002 SEQ ID NO:2 and pPI003 SEQ ID NO:3) showed significant 1,3-butadiene formation after 3 days while no butadiene could be detected with the putative linalool dehydratase from Colletotrichum gloeosporioides (pPI004 SEQ ID NO:4).

FIG. 3: Depicted is the myrcene formation of different variants in 3 h compared to the wild-type enzyme in %. The bars represent the mean values of three independent replicates and the corresponding standard deviation. A high variance was observed in results from replicate experiments in the primary screening in Origami2(DE3).

FIG. 4: Kinetics of the butadiene production of BL21(DE3)_pPI011 (SEQ ID NO:14) revealed a linear increase in butadiene formation over 48 h and the appearance of an unknown peak which also increased over time. Each point of measurement represents one independent reaction.

FIG. 5: GC-analysis of the butadiene formation of BL21(DE3)_pPI011 revealed the appearance of an unknown peak at 1.08 min, which increased over time analogous to the butadiene peak.

FIG. 6: Histogram of the number of LDH mutants and their activity on myrcene formation in % compared to WT (excluding inactive variants). A great number of clones from the second round of engineering showed higher myrcene formation than the wild-type enzyme.

FIG. 7: Comparison of expression and volumetric activity of the linalool dehydratase WT enzyme (pPI010 SEQ ID NO:13) and the variants H83A (pPI011 SEQ ID NO:14) and H252A (pPI012 SEQ ID NO:15) in BL21(DE3) and Origami2(DE3). a) butadiene production in 2d, b) soluble expression. The variants H83A (pPI011) and H252A (pPI012) showed significantly higher soluble expression in Origami2(DE3) than in BL21(DE3) while the wild-type enzyme (pPI010 SEQ ID NO:10) did not.

FIG. 8: Butadiene production within 2 d was tested by using an average cell density of OD600=63. Variants which showed similar butadiene formation compared to the wild-type enzyme are indicated by light grey arrows; the three primary hits are indicated by dark grey arrows. Primary screening of the variants from the first round of engineering in BL21(DE3) revealed three primary hits with increased butadiene production.

FIG. 9: Butadiene formation of the 13 selected variants from the primary screening using the miniature assay; a) absolute values and b) normalized to soluble expression. Normalizing the amount of butadiene formation to the amount of soluble proteins is necessary to distinguish between primary hits which only possess increased soluble expression (e.g. pPI026) and primary hits which possess increased specific activity (e.g. pPI033).

FIG. 10: Confirmation of the results from the miniature assay. a) Butadiene formation of the wild-type (pPI010 SEQ ID NO:10) and the variants pPI033 SEQ ID NO:32, pPI036 SEQ ID NO:15 and pPI037 SEQ ID NO:136 in BL21(DE3) normalized to soluble expression, b) SDS-page of the soluble fractions of the expression cultures used in the assay. Each lane contains the amount of protein which corresponds to OD600=10.

FIGS. 11A and 11B: Butadiene (FIG. 11A) and isoprene (FIG. 11B) production by certain purified mutants.

### DETAILED DESCRIPTION

All references referred to are incorporated herein by reference in their entireties.

Unless specifically defined otherwise, all technical and scientific terms used herein have the same meaning as commonly understood by one of ordinary skill in the art to which this disclosure belongs. Unless mentioned otherwise, the techniques employed or contemplated herein are standard methodologies well known to one of ordinary skill in the art. The practice of the present disclosure will employ, unless otherwise indicated, conventional techniques of microbiology, tissue culture, molecular biology, chemistry, biochemistry and recombinant DNA technology, which are within the skill of the art. The materials, methods and examples are illustrative only and not limiting. The following is presented by way of illustration and is not intended to limit the scope of the disclosure.

Many modifications and other embodiments of the disclosures set forth herein will come to mind to one skilled in the art to which these disclosures pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. Although specific terms are employed herein, they are used in a generic and descriptive sense only and not for purposes of limitation.

Units, prefixes and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. The terms defined below are more fully defined by reference to the specification as a whole.

In the present description and claims, the conventional one-letter and three-letter codes for amino acid residues are used. For ease of reference, the polypeptides as described herein are described by use of the following nomenclature: Original amino acid(s):position(s):substituted amino acid(s) (e.g., A58R, where A is replaced with R at amino acid position 58). All the numbering is with reference to the numbering of wild-type polypeptide of SEQ ID NO: 11.

In the present description and claims, the activity of the claimed polypeptide is measured relative to that of the protein of SEQ ID NO: 10. The numbering of the claimed polypeptide is determined relative to that of the protein of SEQ ID NO: 11. The homology of the polypeptide to the wild-type LDH of SEQ ID NO: 11 is determined without taking into account the presence or lack of a periplasmic tag, and the presence of lack of a poly-His tag.

As used herein, the term “butadiene,” having the molecular formula C₄H₆ and a molecular mass of 54.09 g/mol (IUPAC name Buta-1,3-diene) is used interchangeably with 1,3-butadiene, biethylene, erythrene, divinyl, vinylethylene. Butadiene is a colorless, non-corrosive liquefied gas with a mild aromatic or gasoline-like odor. Butadiene is both explosive and flammable because of its low flash point.

The term “conservatively modified variants” or conservatively modified polypeptides applies to both amino acid and nucleic acid sequences. With respect to particular nucleic acid sequences, conservatively modified variants refer to those nucleic acids that encode identical or conservatively modified variants of the amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also describes every possible silent variation of the nucleic acid. One of ordinary skill will recognize that each codon in a nucleic acid (except AUG, which is ordinarily the only codon for methionine; one exception is Micrococcus rubens, for which GTG is the methionine codon (Ishizuka, et al., (1993) J. Gen. Microbiol. 139:425-32) can be modified to yield a functionally identical molecule. Accordingly, each silent variation of a nucleic acid, which encodes a polypeptide of the present disclosure, is implicit in each described polypeptide sequence and incorporated herein by reference.

As to amino acid sequences, one of skill will recognize that individual substitutions, deletions or additions to a nucleic acid, peptide, polypeptide or protein sequence which alters, adds, or deletes a single amino acid or a small percentage of amino acids in the encoded sequence is a “conservatively modified variant” when the alteration results in the substitution of an amino acid with a chemically similar amino acid. Thus, any number of amino acid residues can be so altered. Conservatively modified variants typically provide equivalent biological activity as the unmodified polypeptide sequence from which they are derived. Conservative substitution tables providing functionally similar amino acids, also referred herein as “equivalent amino acids” are well known in the art.

As used herein, “consisting essentially of” means the inclusion of additional sequences to an object polynucleotide or polypeptide where the additional sequences do not materially affect the basic function of the claimed polynucleotide or polypeptide sequences.

“Codon optimization” is the process of modifying a nucleotide sequence in a manner that improves its expression, G/C content, RNA secondary structure, and translation in eukaryotic cells, without altering the amino acid sequence it encodes. Altered codon usage is often employed to alter translational efficiency and/or to optimize the coding sequence for expression in a desired host or to optimize the codon usage in a heterologous sequence for expression in a particular host. Codon usage in the coding regions of the polynucleotides of the present disclosure can be analyzed statistically using commercially available software packages such as “Codon Preference” available from the University of Wisconsin Genetics Computer Group. See, Devereaux, et al., (1984) Nucleic Acids Res. 12:387-395) or MacVector 4.1 (Eastman Kodak Co., New Haven, Conn.). Thus, the present disclosure provides a codon usage frequency characteristic of the coding region of at least one of the polynucleotides of the present disclosure. The number of polynucleotides (3 nucleotides per amino acid) that can be used to determine a codon usage frequency can be any integer from 3 to the number of polynucleotides of the present disclosure as provided herein. Optionally, the polynucleotides will be full-length sequences. An exemplary number of sequences for statistical analysis can be at least 1, 5, 10, 20, 50 or 100.

The term “derived” encompasses the terms “originated from”, “obtained” or “obtainable from”, and “isolated from”.

“Equivalent amino acids” can be determined either on the basis of their structural homology with the amino acids for which they are substituted or on the results of comparative tests of biological activity between the various polypeptides likely to be generated. As a non-limiting example, the list below summarizes possible substitutions often likely to be carried out without resulting in a significant modification of the biological activity of the corresponding variant:

1) Alanine (A), Serine (S), Threonine (T), Valine (V), Glycine (G), and Proline (P);

2) Aspartic acid (D), Glutamic acid (E);

3) Asparagine (N), Glutamine (Q);

4) Arginine (R), Lysine (K), Histidine (H);

5) Isoleucine (I), Leucine (L), Methionine (M), Valine (V) and

6) Phenylalanine (F), Tyrosine (Y), Tryptophan (W).

See also, Creighton, Proteins, W.H. Freeman and Co. (1984).

In making such changes/substitutions, the hydropathic index of amino acids may also be considered. The importance of the hydropathic amino acid index in conferring interactive biologic function on a protein is generally understood in the art (Kyte and Doolittle, (1982) J Mol Biol. 157(1):105-32). It is accepted that the relative hydropathic character of the amino acid contributes to the secondary structure of the resultant protein, which in turn defines the interaction of the protein with other molecules, for example, enzymes, substrates, receptors, DNA, antibodies, antigens and the like.

It is known in the art that certain amino acids may be substituted by other amino acids having a similar hydropathic index or score and still result in a protein with similar biological activity, i.e., still obtain a biological functionally equivalent protein. Each amino acid has been assigned a hydropathic index on the basis of its hydrophobicity and charge characteristics (Kyte and Doolittle, ibid). These are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9) and arginine (−4.5). In making such changes, the substitution of amino acids whose hydropathic indices are within +2 is preferred, those which are within +1 are particularly preferred and those within +0.5 are even more particularly preferred.

It is also understood in the art that the substitution of like amino acids can be made effectively on the basis of hydrophilicity. U.S. Pat. No. 4,554,101, states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (+3.0); aspartate (+3.0.+0.1); glutamate (+3.0.+0.1); serine (+0.3); asparagine (+0.2); glutamine (+0.2); glycine (0); threonine (−0.4); proline (−0.5.+0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

“Endogenous” with reference to a polynucleotide or protein refers to a polynucleotide or protein that occurs naturally in the host cell.

As used herein, “expression” refers to the process by which a polypeptide is produced based on the nucleic acid sequence of a gene. The process includes both transcription and translation.

An “expression vector” as used herein means a DNA construct comprising a DNA sequence which is operably linked to a suitable control sequence capable of effecting expression of the DNA in a suitable host. Such control sequences may include a promoter to effect transcription, an optional operator sequence to control transcription, a sequence encoding suitable ribosome binding sites on the mRNA, enhancers and sequences which control termination of transcription and translation.

Examples of routinely used “expression systems” include recombinant baculovirus, lentivirus, protozoa (e.g., eukaryotic parasite Leishmania tarentolae), microbial expression systems, including yeast-based (e.g. Pichia Pastoris, Saccharomyces cerevisiae, Yaerobia lipolytica, Hansenula polymorpha, Aspergillus and Trichoderma Fungi) and bacterial-based (e.g. E. coli, Pseudomonas fluorescens, Lactobacillus, Lactococcus, Bacillus megaterium, Bacillus Subtilis, Brevibacillus, Corynebacterium glutamicum), Chinese hamster ovary (CHO) cells, CHOK1 SVNSO (Lonza), BHK (baby hamster kidney), PerC.6 or Per. C6 (e.g., Percivia, Crucell), different lines of HEK 293, Expi293F™ cells (Life Technologies), GenScript's YeastHIGH™ Technology (GenScript), human neuronal precursor cell line AGE1.HN (Probiogen) and other mammalian cells, plants (e.g., corn, alfalfa, and tobacco), insect cells, avian eggs, algae, and transgenic animals (e.g., mice, rats, goats, sheep, pigs, cows). The advantages and disadvantages of these various systems have been reviewed in the literature and are known to one of ordinary skill in the art.

A “gene” refers to a DNA segment that is involved in producing a polypeptide and includes regions preceding and following the coding regions as well as intervening sequences (introns) between individual coding segments (exons).

“Host strain” or “host cell” means a suitable host for an expression vector or DNA construct comprising a polynucleotide encoding a LDH enzyme according to the disclosure. Specifically, host strains may be bacterial cells, mammalian cells, insect cells, and other cloning or “expression systems.” In an embodiment of the disclosure, “host cell” means both the cells and protoplasts created from the cells of a microbial strain. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

“Heterologous” with reference to a polynucleotide or protein refers to a polynucleotide or protein that does not naturally occur in a host cell. In some embodiments, the protein is a commercially important industrial protein. It is intended that the term encompass proteins that are encoded by naturally occurring genes, mutated genes, and/or synthetic genes.

A polynucleotide or a polypeptide having a certain percent (e.g., at least 90%, 91%, 92%, 93%, 94%, 95%, 96%, 97%, 98%, or 99%) of sequence identity with another sequence means that, when aligned, that percentage of bases or amino acid residues are the same in comparing the two sequences. Identity can be substitute homology in alternate embodiments of the disclosed and claimed peptides. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution and this process results in “sequence homology” of, e.g, at least 90%, 91%, 92%, 93%, 94%, 95%, 96%, 97%, 98%, or 99%. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, (1988) Computer Applic. Biol. Sci. 4:11-17, e.g., as implemented in the program PC/GENE (Intelligenetics, Mountain View, Calif., USA). This alignment and the percent homology or identity can be determined using any suitable software program known in the art, for example those described in CURRENT PROTOCOLS IN MOLECULAR BIOLOGY (F. M. Ausubel et al. (eds) 1987, Supplement 30, section 7.7.18). Such programs may include the GCG Pileup program, FASTA (Pearson et al. (1988) Proc. Natl, Acad. Sci USA 85:2444-2448), and BLAST (BLAST Manual, Altschul et al., Nat'l Cent. Biotechnol. Inf., Natl Lib. Med. (NCIB NLM NIH), Bethesda, Md., and Altschul et al., (1997) NAR 25:3389-3402). Another alignment program is ALIGN Plus (Scientific and Educational Software, Pa.), using default parameters. Another sequence software program that finds use is the TFASTA Data Searching Program available in the Sequence Software Package Version 6.0 (Genetics Computer Group, University of Wisconsin, Madison, Wis.).

“Introduced” in the context of inserting a nucleic acid sequence into a cell, means “transfection”, or “transformation” or “transduction” and includes reference to the incorporation of a nucleic acid sequence into a eukaryotic or prokaryotic cell wherein the nucleic acid sequence may be incorporated into the genome of the cell (e.g., chromosome, plasmid, plastid, or mitochondrial DNA), converted into an autonomous replicon, or transiently expressed (e.g., transfected mRNA).

As used herein, “nucleotide sequence” or “nucleic acid sequence” refers to an oligonucleotide sequence or polynucleotide sequence and variants, homologues, fragments and derivatives thereof. The nucleotide sequence may be of genomic, synthetic or recombinant origin and may be double-stranded or single-stranded, whether representing the sense or anti-sense strand. As used herein, the term “nucleotide sequence” includes genomic DNA, cDNA, synthetic DNA, and RNA.

The term “nucleic acid” encompasses DNA, cDNA, RNA, heteroduplexes, and synthetic molecules capable of encoding a polypeptide. RNA includes mRNA, RNA, RNAi, siRNA, cRNA and autocatalytic RNA. Nucleic acids may be single stranded or double stranded, and may be chemical modifications. The terms “nucleic acid” and “polynucleotide” are used interchangeably. Because the genetic code is degenerate, more than one codon may be used to encode a particular amino acid, and the present compositions and methods encompass nucleotide sequences which encode a particular amino acid sequence. A nucleic acid comprises a nucleotide sequence which typically includes nucleotides that comprise an A, G, C, T or U base. However, nucleotide sequences may include other bases such as, without limitation inosine, methylycytosine, methylinosine, methyladenosine and/or thiouridine, although without limitation thereto.

One skilled in the art will recognize that nucleic acid sequences encompassed by the disclosure are also defined by the ability to hybridize under stringent hybridization conditions with nucleic acid sequences encoding the exemplified LDH variants. A nucleic acid is hybridizable to another nucleic acid sequence when a single stranded form of the nucleic acid can anneal to the other nucleic acid under appropriate conditions of temperature and solution ionic strength. Hybridization and washing conditions are well known in the art (Sambrook, et al. (Molecular cloning: a laboratory manual, Cold Spring Harbor Laboratory; 4th edition, 2012). Hybridization under highly stringent conditions means that conditions related to temperature and ionic strength are selected in such a way that they allow hybridization to be maintained between two complementarity DNA fragments. On a purely illustrative basis, the highly stringent conditions of the hybridization step for the purpose of defining the polynucleotide fragments described above are advantageously as follows.

DNA-DNA or DNA-RNA hybridization is carried out in two steps: (1) prehybridization at 42° C. for three hours in phosphate buffer (20 mM, pH 7.5) containing 5×SSC (1×SSC corresponds to a solution of 0.15 M NaCl+0.015 M sodium citrate), 50% formamide, 7% sodium dodecyl sulfate (SDS), 10×Denhardt's, 5% dextran sulfate and 1% salmon sperm DNA; (2) primary hybridization for 20 hours at a temperature depending on the length of the probe (i.e.: 42° C. for a probe>100 nucleotides in length) followed by two 20-minute washings at 20° C. in 2×SSC+2% SDS, one 20-minute washing at 20° C. in 0.1×SSC+0.1% SDS. The last washing is carried out in 0.1×SSC+0.1% SDS for 30 minutes at 60° C. for a probe>100 nucleotides in length. The highly stringent hybridization conditions described above for a polynucleotide of defined size can be adapted by a person skilled in the art for longer or shorter oligonucleotides, according to the procedures described in Sambrook, et al. (Molecular cloning: a laboratory manual, Cold Spring Harbor Laboratory; 3rd edition, 2001).

Stringent conditions may also be achieved with the addition of destabilizing agents such as formamide.

The term “operably linked” and its variants refer to chemical fusion or bonding or association of sufficient stability to withstand conditions encountered in the nucleotide incorporation methods utilized, between a combination of different compounds, molecules or other entities such as, but not limited to: between a mutant polymerase and a reporter moiety (e.g., fluorescent dye or nanoparticle); between a nucleotide and a reporter moiety (e.g., fluorescent dye); or between a promoter and a coding sequence (e.g., one encoding one of the polypeptides disclosed herein), if it controls the transcription of the sequence.

A “promoter” is a regulatory sequence that is involved in binding RNA polymerase to initiate transcription of a gene. The promoter may be an inducible promoter or a constitutive promoter. An exemplary promoter used herein is a T7 promoter, which is an inducible promoter.

A “periplasmic tag” or “periplasmic leader sequence” is a sequence of amino acids which, when attached to/present at the N-terminus of a protein/peptide, directs the protein/peptide to the bacterial periplasm, where the sequence is often removed by a signal peptidase. Protein/peptide secretion into the periplasm can increase the stability of recombinantly-expressed proteins/peptides.

“Recombinant” when used in reference to a cell, nucleic acid, protein or vector, indicates that the cell, nucleic acid, protein or vector, has been modified by the introduction of a “heterologous nucleic acid” or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

A Origami2(DE3)-“related strain” or a BL21(DE3)-related strain is a strain that has essentially the same functional properties and/or advantages in recombinant protein expression as do Origami2(DE3) or BL21(DE3) bacteria, respectively.

A “signal sequence” or “signal peptide” means a sequence of amino acids bound to the N-terminal portion of a protein, which facilitates the secretion of the mature form of the protein outside the cell. The definition of a signal sequence is a functional one. The mature form of the extracellular protein lacks the signal sequence which is cleaved off during the secretion process.

“Selective marker” refers to a gene capable of expression in a host that allows for ease of selection of those hosts containing an introduced nucleic acid or vector. Examples of selectable markers include but are not limited to antimicrobials (e.g., hygromycin, bleomycin, or chloramphenicol) and/or genes that confer a metabolic advantage, such as a nutritional advantage on the host cell.

“Under transcriptional control” is a term well understood in the art that indicates that transcription of a polynucleotide sequence, usually a DNA sequence, depends on its being operably linked to an element which contributes to the initiation of, or promotes transcription.

“Under translational control” is a term well understood in the art that indicates a regulatory process that occurs after mRNA has been formed.

As used herein, “transformed cell” includes cells that have been transformed or transduced by use of recombinant DNA techniques. Transformation typically occurs by insertion of one or more nucleotide sequences into a cell. The inserted nucleotide sequence may be a “heterologous nucleotide sequence,” i.e., is a sequence that is not natural to the cell that is to be transformed, such as a fusion protein.

As used herein, “transformed”, “stably transformed”, “transduced,” and “transgenic” used in reference to a cell means the cell has a non-native (e.g., heterologous) nucleic acid sequence integrated into its genome or as an episomal plasmid that is maintained through multiple generations.

“Variants” refer to both polypeptides and nucleic acids that are different from a related wild-type sequence. The term “variant” may be used interchangeably with the term “mutant.” Variants include insertions, substitutions, transversions, truncations, and/or inversions at one or more locations in the amino acid or nucleotide sequence, respectively, of a parent sequence. Variant nucleic acids can include sequences that are complementary to sequences that are capable of hybridizing to the nucleotide sequences presented herein. For example, a variant sequence is complementary to sequences capable of hybridizing under stringent conditions (e.g., 50° C. and 0.2.×SSC (1×SSC=0.15 M NaCl, 0.015 M sodium citrate, pH 7.0)) to the nucleotide sequences presented herein. More particularly, the term variant encompasses sequences that are complementary to sequences that are capable of hybridizing under highly stringent conditions (e.g., 65° C. and 0.1×SSC) to the nucleotide sequences presented herein. In one embodiment, certain polypeptides described herein can be said to be variants of wild-type LDH from Castellaniella defragrans.

The term “vector”, as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the claimed embodiments are intended to include such other forms of expression vectors, such as viral vectors (e.g., replication defective retroviruses, adenoviruses and adeno-associated viruses), which serve equivalent functions. Vectors also include cloning vectors, shuttle vectors, plasmids, phage particles, cassettes and the like.

Reference will now be made in detail to various disclosed embodiments. In the present description and claims, the newly disclosed polypeptides have improved activity in the catalysis of the dehydration of 3-buten-2-ol to 1,3-butadiene relative to wild-type LDH of Castellaniella defragrans. In the present description and claims, the activity of the claimed variant is measured relative to that of the protein of SEQ ID NO: 13. The numbering of the claimed variant is determined relative to that of the protein of SEQ ID NO: 11. The homology of the variant to the parent LDH is determined without taking into account the presence or lack of a periplasmic tag, and the presence of lack of a poly-His tag. Where it is said that the variant has one, two, or three alterations relative to the parent LDH of SEQ ID NO: 11, the presence or lack of a periplasmic tag, and the presence or lack of a poly-His tag are not taken into account when counting the variations. Furthermore, where it is said that the variant has one, two, or three alterations relative to the parent LDH of SEQ ID NO: 11, it is the same as saying that the variant has one, two, or three alterations relative to the mature form of the parent LDH of SEQ ID NO: 11 (i.e, the protein of SEQ ID NO:11 without the signal peptide, underlined in the protein sequence of the Sequence Listing).

Altered Properties of the Disclosed Variants

The following discusses the relationship between mutations that may be present in the polypeptides provided herein, and desirable alterations in properties (relative to those of the wild-type parent LDH of SEQ ID NO: 11, 13, 37, OR 38). These properties include: improved solubility, improved 3-buten-2-ol dehydratase activity, polypeptides with improved catalytic activity in the conversion of 3-methyl-3-buten-2-ol to isoprene.

Improved Solubility

In one embodiment, polypeptides with improved solubility are provided. Improved solubility can be measured by any method known to one of ordinary skill in the art. In one embodiment, the claimed polypeptides have improved solubility as measured by at least one assay. In one embodiment, improved solubility of a polypeptide refers to an increased level of protein in the soluble fraction of a bacterial cell extract, relative to the wild-type LDH (SEQ ID NO: 11, 13, 37, OR 38).

In some embodiments, the solubility is at least 80% of that of a polypeptide consisting of SEQ ID NO: 11, 13, 37, OR 38. In some embodiments, the solubility is increased about 1.5 fold or greater when compared to that of a polypeptide consisting of SEQ ID NO: 11, 13, 37, OR 38. In some embodiments, the solubility is increased about 2 fold or greater when compared to that of a polypeptide consisting of SEQ ID NO: 11, 13, 37, OR 38. In some embodiments, the solubility is increased about 2.5 fold or greater when compared to that of a polypeptide consisting of SEQ ID NO: 11, 13, 37, OR 38. In some embodiments, the solubility is increased about 3 fold or greater when compared to that of a polypeptide consisting of SEQ ID NO: 11, 13, 37, OR 38. In some embodiments, the solubility is increased about 3.5 fold or greater when compared to that of a polypeptide consisting of SEQ ID NO: 11, 13, 37, OR 38. In some embodiments, the solubility is increased about 4 fold or greater when compared to that of a polypeptide consisting of SEQ ID NO: 11, 13, 37, OR 38. In some embodiments, the solubility is increased about 4.5 fold or greater when compared to that of a polypeptide consisting of SEQ ID NO: 11, 13, 37, OR 38. In some embodiments, the solubility is increased about 5 fold or greater when compared to that of a polypeptide consisting of SEQ ID NO: 11, 13, 37, OR 38. All of these embodiments can be combined with any of the embodiments described below.

In some embodiments, the increased solubility is observed in at least one type of non-bacterial cells. In some embodiments, the increased solubility is observed in at least one type of bacteria. In some embodiments, the increased solubility is observed in more than one type of bacteria. In some embodiments, the bacteria are a strain of E. coli. In some embodiments, the bacteria are Origami2(DE3) or a related strain. In some embodiments, the bacteria are BL21(DE3) or a related strain. In some embodiments, the solubility of the peptide is increased when compared to that of a polypeptide consisting of SEQ ID NO: 11, 13, 37, OR 38.

One embodiment provides a polypeptide wherein:
a) the polypeptide has increased solubility relative to that of a polypeptide consisting of SEQ ID NO: 11, 13, 37, OR 38 in at least one cell type;
b) the polypeptide comprises an amino acid sequence with at least 90% amino acid sequence homology to SEQ ID NO:11
c) the polypeptide comprises 1-3 alteration(s) relative to SEQ ID NO:11 independently selected from:
   a substitution of the amino acid that occupies position 58 with a different amino acid selected from R and equivalent amino acids;
   a substitution of the amino acid that occupies position 83 with a different amino acid selected from A and equivalent amino acids; and
   a substitution of the amino acid that occupies position 252 with a different amino acid selected from A and equivalent amino acids.

In related embodiments, the polypeptide comprises an amino acid sequence with at least 91% amino acid sequence homology to SEQ ID NO:11. In related embodiments, the polypeptide comprises an amino acid sequence with at least 92% amino acid sequence homology to SEQ ID NO:11. In related embodiments, the polypeptide comprises an amino acid sequence with at least 93% amino acid sequence homology to SEQ ID NO:11. In related embodiments, the polypeptide comprises an amino acid sequence with at least 94% amino acid sequence homology to SEQ ID NO:11. In related embodiments, the polypeptide comprises an amino acid sequence with at least 95% amino acid sequence homology to SEQ ID NO:11. In related embodiments, the polypeptide comprises an amino acid sequence with at least 96% amino acid sequence homology to SEQ ID NO:11. In related embodiments, the polypeptide comprises an amino acid sequence with at least 97% amino acid sequence homology to SEQ ID NO:11. In related embodiments, the polypeptide comprises an amino acid sequence with at least 98% amino acid sequence homology to SEQ ID NO:11. In related embodiments, the polypeptide comprises an amino acid sequence with at least 99% amino acid sequence homology to SEQ ID NO:11.

In further related embodiments, the polypeptide has one alteration relative to SEQ ID NO: 11 selected from:
a substitution of the amino acid that occupies position 58 with a different amino acid selected from R and equivalent amino acids;
a substitution of the amino acid that occupies position 83 with a different amino acid selected from A and equivalent amino acids; and
a substitution of the amino acid that occupies position 252 with a different amino acid selected from A and equivalent amino acids.

In further related embodiments, the polypeptide has two alterations relative to SEQ ID NO:11 selected from:
a substitution of the amino acid that occupies position 58 with a different amino acid selected from R and equivalent amino acids;
a substitution of the amino acid that occupies position 83 with a different amino acid selected from A and equivalent amino acids; and
a substitution of the amino acid that occupies position 252 with a different amino acid selected from A and equivalent amino acids.

In further related embodiments, the polypeptide has three alterations relative to SEQ ID NO:11, namely:
a substitution of the amino acid that occupies position 58 with a different amino acid selected from R and equivalent amino acids;
a substitution of the amino acid that occupies position 83 with a different amino acid selected from A and equivalent amino acids; and
a substitution of the amino acid that occupies position 252 with a different amino acid selected from A and equivalent amino acids.

In a further related embodiment, the polypeptide has a substitution of the amino acid that occupies position 58 with a different amino acid selected from R and equivalent amino acids. In a further related embodiment, the polypeptide has a substitution of the amino acid that occupies position 83 with a different amino acid selected from A and equivalent amino acids. In a further related embodiment, the polypeptide has a substitution of the amino acid that occupies position 252 with a different amino acid selected from A and equivalent amino acids. Any of these polypeptides may comprise an amino acid sequence with at least 90, at least 91, at least 92, at least 93, at least 94, at least 95, at least 96, at least 97, at least 98, or at least 99% amino acid sequence homology to SEQ ID NO:11.

In a further related embodiment, the polypeptide has a A58R substitution. In a further related embodiment, the polypeptide has a H83A substitution. In a further related embodiment, the polypeptide has a H252A substitution. In a further related embodiment, the polypeptide has a A58R substitution and a H83A substitution. In a further related embodiment, the polypeptide has a A58R substitution and a H252A substitution. In a further related embodiment, the polypeptide has a H83A substitution and a H252A substitution. Any of these polypeptide s may comprise an amino acid sequence with at least 90, at least 91, at least 92, at least 93, at least 94, at least 95, at least 96, at least 97, at least 98, or at least 99% amino acid sequence homology to SEQ ID NO:11.

In a further related embodiment, the polypeptide has a A58R substitution, a H83A substitution, and a H252A substitution. In various embodiments, this polypeptide may comprise an amino acid sequence with at least 90, at least 91, at least 92, at least 93, at least 94, at least 95, at least 96, at least 97, at least 98, or at least 99% amino acid sequence homology to SEQ ID NO:11.

Improved Increased Specific Activity in the Catalysis of the Dehydration of 3-buten-2-ol to 1,3-butadiene

Some other embodiments provide polypeptides improved specific activity in the catalysis of the dehydration of 3-buten-2-ol to 1,3-butadiene, relative to that of a polypeptide consisting of SEQ ID NO: 11, 13, 37, OR 38. Improved specific activity in the catalysis of the dehydration of 3-buten-2-ol to 1,3-butadiene can be measured by any method known to one of ordinary skill in the art. In one embodiment, improved specific activity in the catalysis of the dehydration of 3-buten-2-ol to 1,3-butadiene of polypeptide refers to an increased specific activity in the catalysis of the dehydration of 3-buten-2-ol to 1,3-butadiene as measured by at least one assay. In one embodiment, improved specific activity in the catalysis of the dehydration of 3-buten-2-ol to 1,3-butadiene of polypeptide refers to an increased specific activity in the catalysis of the dehydration of 3-buten-2-ol to 1,3-butadiene of polypeptide isolated from a bacterial cell extract, relative to the similarly isolated polypeptide consisting of SEQ ID NO: 11, 13, 37, OR 38. In another embodiment, improved specific activity in the catalysis of the dehydration of 3-buten-2-ol to 1,3-butadiene of a polypeptide refers to an increased specific activity in the catalysis of the dehydration of 3-buten-2-ol to 1,3-butadiene of a bacterial cell extract/lyzate expressing a polypeptide, relative to a bacterial cell extract expressing a polypeptide consisting of SEQ ID NO: 11, 13, 37, OR 38.

In some embodiments, the specific activity is at least 80% of that of a polypeptide consisting of SEQ ID NO: 11, 13, 37, OR 38. In some embodiments, the specific activity in the catalysis of the dehydration of 3-buten-2-ol to 1,3-butadiene is increased about 1.5 fold or greater when compared to that of a polypeptide consisting of SEQ ID NO: 11, 13, 37, OR 38. In some embodiments, the specific activity in the catalysis of the dehydration of 3-buten-2-ol to 1,3-butadiene is increased about 2 fold or greater when compared to that of a polypeptide consisting of SEQ ID NO: 11, 13, 37, OR 38. In some embodiments, the specific activity in the catalysis of the dehydration of 3-buten-2-ol to 1,3-butadiene is increased about 2.5 fold or greater when compared to that of a polypeptide consisting of SEQ ID NO: 11, 13, 37, OR 38. In some embodiments, the specific activity in the catalysis of the dehydration of 3-buten-2-ol to 1,3-butadiene is increased about 3 fold or greater when compared to that of a polypeptide consisting of SEQ ID NO: 11, 13, 37, OR 38. In some embodiments, the specific activity in the catalysis of the dehydration of 3-buten-2-ol to 1,3-butadiene is increased about 3.5 fold or greater when compared to that of a polypeptide consisting of SEQ ID NO: 11, 13, 37, OR 38. In some embodiments, the specific activity in the catalysis of the dehydration of 3-buten-2-ol to 1,3-butadiene is increased about 4 fold or greater when compared to that of a polypeptide consisting of SEQ ID NO: 11, 13, 37, OR 38. In some embodiments, the specific activity in the catalysis of the dehydration of 3-buten-2-ol to 1,3-butadiene is increased about 4.5 fold or greater when compared to that of a polypeptide consisting of SEQ ID NO: 11, 13, 37, OR 38. In some embodiments, the specific activity in the catalysis of the dehydration of 3-buten-2-ol to 1,3-butadiene is increased about 5 fold or greater when compared to that of a polypeptide consisting of SEQ ID NO: 11, 13, 37, OR 38. All of these embodiments can be combined with any of the embodiments described below.

In some embodiments, the increase in the catalysis of the dehydration of 3-buten-2-ol to 1,3-butadiene is observed in at least one type of non-bacterial cells expressing a polypeptide consisting of SEQ ID NO: 11, 13, 37, OR 38. In some embodiments, the increased in the catalysis of the dehydration of 3-buten-2-ol to 1,3-butadiene is observed in at least one type of bacteria. In some embodiments, the increased in the catalysis of the dehydration of 3-buten-2-ol to 1,3-butadiene is observed in more than one type of bacteria. In some embodiments, the bacteria are a strain of E. coli. In some embodiments, the bacteria are Origami2(DE3) or a related strain. In some embodiments, the bacteria are BL21(DE3) or a related strain.

One embodiment provides an isolated polypeptide wherein:
a) the polypeptide has improved specific activity in the catalysis of the dehydration of 3-buten-2-ol to 1,3-butadiene relative to that of a polypeptide consisting of SEQ ID NO: 11, 13, 37, OR 38 in at least one cell type;
b) the polypeptide comprises an amino acid sequence with at least 90% amino acid sequence homology to SEQ ID NO:11
c) the polypeptide comprises 1-3 alteration(s) relative to SEQ ID NO:11 independently selected from:
   a substitution of the amino acid that occupies position 168 with a different amino acid selected from D and equivalent amino acids;
   a substitution of the amino acid that occupies position 230 with a different amino acid selected from E and equivalent amino acids; and
   a substitution of the amino acid that occupies position 366 with a different amino acid selected from V and equivalent amino acids.

In related embodiments, the polypeptide comprises an amino acid sequence with at least 91% amino acid sequence homology to SEQ ID NO:11. In related embodiments, the polypeptide comprises an amino acid sequence with at least 92% amino acid sequence homology to SEQ ID NO:11. In related embodiments, the polypeptide comprises an amino acid sequence with at least 93% amino acid sequence homology to SEQ ID NO:11. In related embodiments, the polypeptide comprises an amino acid sequence with at least 94% amino acid sequence homology to SEQ ID NO:11. In related embodiments, the polypeptide comprises an amino acid sequence with at least 95% amino acid sequence homology to SEQ ID NO:11. In related embodiments, the polypeptide comprises an amino acid sequence with at least 96% amino acid sequence homology to SEQ ID NO:11. In related embodiments, the polypeptide comprises an amino acid sequence with at least 97% amino acid sequence homology to SEQ ID NO:11. In related embodiments, the polypeptide comprises an amino acid sequence with at least 98% amino acid sequence homology to SEQ ID NO:11. In related embodiments, the polypeptide comprises an amino acid sequence with at least 99% amino acid sequence homology to SEQ ID NO:11.

In further related embodiments, the polypeptide has one alteration relative to SEQ ID NO:11 selected from:
a substitution of the amino acid that occupies position 168 with a different amino acid selected from D and equivalent amino acids;
a substitution of the amino acid that occupies position 230 with a different amino acid selected from E and equivalent amino acids; and
a substitution of the amino acid that occupies position 366 with a different amino acid selected from V and equivalent amino acids.

In further related embodiments, the polypeptide has two alterations relative to SEQ ID NO:11 selected from:
a substitution of the amino acid that occupies position 168 with a different amino acid selected from D and equivalent amino acids;
a substitution of the amino acid that occupies position 230 with a different amino acid selected from E and equivalent amino acids; and
a substitution of the amino acid that occupies position 366 with a different amino acid selected from V and equivalent amino acids.

In further related embodiments, the polypeptide has three alterations relative to SEQ ID NO:11, namely:
a substitution of the amino acid that occupies position 168 with a different amino acid selected from D and equivalent amino acids;
a substitution of the amino acid that occupies position 230 with a different amino acid selected from E and equivalent amino acids; and
a substitution of the amino acid that occupies position 366 with a different amino acid selected from V and equivalent amino acids.

In a further related embodiment, the polypeptide has a substitution of the amino acid that occupies position 168 with a different amino acid selected from D and equivalent amino acids. In a further related embodiment, the polypeptide has a substitution of the amino acid that occupies position 230 with a different amino acid selected from E and equivalent amino acids. In a further related embodiment, the polypeptide has a substitution of the amino acid that occupies position 366 with a different amino acid selected from V and equivalent amino acids. Any of these polypeptides may comprise an amino acid sequence with at least 90, at least 91, at least 92, at least 93, at least 94, at least 95, at least 96, at least 97, at least 98, or at least 99% amino acid sequence homology to SEQ ID NO:11.

In a further related embodiment, the polypeptide has a S168D substitution. In a further related embodiment, the polypeptide has a A230E substitution. In a further related embodiment, the polypeptide has a L366V substitution. In a further related embodiment, the polypeptide has a S168D substitution and a A230E substitution. In a further related embodiment, the polypeptide has a S168D substitution and a L366V substitution. In a further related embodiment, the polypeptide has a A230E substitution and a L366V substitution. Any of these polypeptides may comprise an amino acid sequence with at least 90, at least 91, at least 92, at least 93, at least 94, at least 95, at least 96, at least 97, at least 98, or at least 99% amino acid sequence homology to SEQ ID NO:11.

In a further related embodiment, the polypeptide has a S168D substitution, a A230E substitution, and a L366V substitution. In various embodiments, this polypeptide may comprise an amino acid sequence with at least 90, at least 91, at least 92, at least 93, at least 94, at least 95, at least 96, at least 97, at least 98, or at least 99% amino acid sequence homology to SEQ ID NO:11.

Improved Activity in the Catalysis of the Dehydration of Linalool to Myrcene

Some other embodiments provide polypeptides improved specific activity in the catalysis of the dehydration of linalool to myrcene, relative to that of a polypeptide consisting of SEQ ID NO: 11, 13, 37, OR 38. Improved specific activity in the catalysis of the dehydration of linalool to myrcene can be measured by any method known to one of ordinary skill in the art. In one embodiment, the polypeptide has improved specific activity in the catalysis of the dehydration of linalool to myrcene as measured by at least one method. In one embodiment, improved specific activity in the catalysis of the dehydration of linalool to myrcene of polypeptide refers to an increased specific activity in the catalysis of the dehydration of linalool to myrcene of polypeptide isolated from a bacterial cell extract, relative to the similarly isolated polypeptide consisting of SEQ ID NO: 11, 13, 37, OR 38. In another embodiment, improved specific activity in the catalysis of the dehydration of linalool to myrcene of an polypeptide refers to an increased specific activity in the catalysis of the dehydration of linalool to myrcene of a bacterial cell extract/lyzate expressing a polypeptide, relative to a bacterial cell extract expressing a polypeptide consisting of SEQ ID NO: 11, 13, 37, OR 38.

In some embodiments, the specific activity is at least 80% of that of a polypeptide consisting of SEQ ID NO: 11, 13, 37, OR 38. In some embodiments, the specific activity in the catalysis of the dehydration of linalool to myrcene is increased about 1.5 fold or greater when compared to that of a polypeptide consisting of SEQ ID NO: 11, 13, 37, OR 38. In some embodiments, the specific activity in the catalysis of the dehydration of linalool to myrcene is increased about 2 fold or greater when compared to that of a polypeptide consisting of SEQ ID NO: 11, 13, 37, OR 38. In some embodiments, the specific activity in the catalysis of the dehydration of linalool to myrcene is increased about 2.5 fold or greater when compared to that of a polypeptide consisting of SEQ ID NO: 11, 13, 37, OR 38. In some embodiments, the specific activity in the catalysis of the dehydration of linalool to myrcene is increased about 3 fold or greater when compared to that of a polypeptide consisting of SEQ ID NO: 11, 13, 37, OR 38. In some embodiments, the specific activity in the catalysis of the dehydration of linalool to myrcene is increased about 3.5 fold or greater when compared to that of a polypeptide consisting of SEQ ID NO: 11, 13, 37, OR 38. In some embodiments, the specific activity in the catalysis of the dehydration of linalool to myrcene is increased about 4 fold or greater when compared to that of a polypeptide consisting of SEQ ID NO: 11, 13, 37, OR 38. In some embodiments, the specific activity in the catalysis of the dehydration of linalool to myrcene is increased about 4.5 fold or greater when compared to that of a polypeptide consisting of SEQ ID NO: 11, 13, 37, OR 38. In some embodiments, the specific activity in the catalysis of the dehydration of linalool to myrcene is increased about 5 fold or greater when compared to that of a polypeptide consisting of SEQ ID NO: 11, 13, 37, OR 38. All of these embodiments can be combined with any of the embodiments described below.

In some embodiments, the increase in the catalysis of the dehydration of linalool to myrcene is observed in at least one type of non-bacterial cells expressing a polypeptide consisting of SEQ ID NO: 11, 13, 37, OR 38. In some embodiments, the increased in the catalysis of the dehydration of 3-buten-2-ol to 1,3-butadiene is observed in at least one type of bacteria. In some embodiments, the increased in the catalysis of the dehydration of 3-buten-2-ol to 1,3-butadiene is observed in more than one type of bacteria. In some embodiments, the bacteria are a strain of E. coli. In some embodiments, the bacteria are Origami2(DE3) or a related strain. In some embodiments, the bacteria are BL21(DE3) or a related strain.

One embodiment provides an isolated polypeptide wherein:
a) the polypeptide has improved specific activity in the catalysis of the dehydration of linalool to myrcene relative to that of a polypeptide consisting of SEQ ID NO:13 in at least one cell type;
b) the polypeptide comprises an amino acid sequence with at least 90% amino acid sequence homology to SEQ ID NO:11
c) the polypeptide comprises at least 1-9 alteration(s) relative to SEQ ID NO:11 independently selected from:
   a substitution of the amino acid that occupies position 83 with a different amino acid selected from A and equivalent amino acids;
   a substitution of the amino acid that occupies position 169 with a different amino acid selected from S, G, H, D and equivalent amino acids;
   a substitution of the amino acid that occupies position 186 with a different amino acid selected from C, M and equivalent amino acids; and
   a substitution of the amino acid that occupies position 359 with a different amino acid selected from S, L and equivalent amino acids.

In related embodiments, the polypeptide of the previous paragraph comprises an amino acid sequence with at least 91% amino acid sequence homology to SEQ ID NO:11. In related embodiments, the polypeptide comprises an amino acid sequence with at least 92% amino acid sequence homology to SEQ ID NO:11. In related embodiments, the polypeptide comprises an amino acid sequence with at least 93% amino acid sequence homology to SEQ ID NO:11. In related embodiments, the polypeptide comprises an amino acid sequence with at least 94% amino acid sequence homology to SEQ ID NO:11. In related embodiments, the polypeptide comprises an amino acid sequence with at least 95% amino acid sequence homology to SEQ ID NO:11. In related embodiments, the polypeptide comprises an amino acid sequence with at least 96% amino acid sequence homology to SEQ ID NO:11. In related embodiments, the polypeptide comprises an amino acid sequence with at least 97% amino acid sequence homology to SEQ ID NO:11. In related embodiments, the polypeptide comprises an amino acid sequence with at least 98% amino acid sequence homology to SEQ ID NO:11. In related embodiments, the polypeptide comprises an amino acid sequence with at least 99% amino acid sequence homology to SEQ ID NO:11.

One embodiment provides an isolated polypeptide wherein:
a) the polypeptide comprises an amino acid sequence with at least 90% amino acid sequence homology to SEQ ID NO:11; and
b) the polypeptide comprises 1-9 alteration(s) relative to SEQ ID NO:11 independently selected from:
   a substitution of the amino acid that occupies position 83 with a different amino acid selected from A and equivalent amino acids;
   a substitution of the amino acid that occupies position 169 with a different amino acid selected from S, G, H, D and equivalent amino acids;
   a substitution of the amino acid that occupies position 186 with a different amino acid selected from C, M and equivalent amino acids; and a substitution of the amino acid that occupies position 359 with a different amino acid selected from S, L and equivalent amino acids.

In related embodiments, the polypeptide of the previous paragraph comprises an amino acid sequence with at least 91% amino acid sequence homology to SEQ ID NO:11. In related embodiments, the polypeptide comprises an amino acid sequence with at least 92% amino acid sequence homology to SEQ ID NO:11. In related embodiments, the polypeptide comprises an amino acid sequence with at least 93% amino acid sequence homology to SEQ ID NO:11. In related embodiments, the polypeptide comprises an amino acid sequence with at least 94% amino acid sequence homology to SEQ ID NO:11. In related embodiments, the polypeptide comprises an amino acid sequence with at least 95% amino acid sequence homology to SEQ ID NO:11. In related embodiments, the polypeptide comprises an amino acid sequence with at least 96% amino acid sequence homology to SEQ ID NO:11. In related embodiments, the polypeptide comprises an amino acid sequence with at least 97% amino acid sequence homology to SEQ ID NO:11. In related embodiments, the polypeptide comprises an amino acid sequence with at least 98% amino acid sequence homology to SEQ ID NO:11. In related embodiments, the polypeptide comprises an amino acid sequence with at least 99% amino acid sequence homology to SEQ ID NO:11.

Another embodiment provides a polypeptide of any of the previous four paragraphs that has at least one, at least two, at least three, at least four, at least five, at least six, at least seven, at least eight, or at least 9 alteration relative to SEQ ID NO:11 selected from:
a substitution of the amino acid that occupies position 83 with a different amino acid selected from A and equivalent amino acids;
a substitution of the amino acid that occupies position 169 with a different amino acid selected from S, G, H, D and equivalent amino acids;
a substitution of the amino acid that occupies position 186 with a different amino acid selected from C, M and equivalent amino acids; and
a substitution of the amino acid that occupies position 359 with a different amino acid selected from S, L and equivalent amino acids.

In further related embodiments, the polypeptide has one alteration relative to SEQ ID NO:11 selected from:
a substitution of the amino acid that occupies position 83 with a different amino acid selected from A and equivalent amino acids;
a substitution of the amino acid that occupies position 169 with a different amino acid selected from S, G, H, D and equivalent amino acids;
a substitution of the amino acid that occupies position 186 with a different amino acid selected from C, M and equivalent amino acids; and
a substitution of the amino acid that occupies position 359 with a different amino acid selected from S, L and equivalent amino acids.

In further related embodiments, the polypeptide has two alterations relative to SEQ ID NO:11 selected from:
a substitution of the amino acid that occupies position 83 with a different amino acid selected from A and equivalent amino acids;
a substitution of the amino acid that occupies position 169 with a different amino acid selected from S, G, H, D and equivalent amino acids;
a substitution of the amino acid that occupies position 186 with a different amino acid selected from C, M and equivalent amino acids; and
a substitution of the amino acid that occupies position 359 with a different amino acid selected from S, L and equivalent amino acids.

In further related embodiments, the polypeptide has three alterations relative to SEQ ID NO:11 selected from:
a substitution of the amino acid that occupies position 83 with a different amino acid selected from A and equivalent amino acids;
a substitution of the amino acid that occupies position 169 with a different amino acid selected from S, G, H, D and equivalent amino acids;
a substitution of the amino acid that occupies position 186 with a different amino acid selected from C, M and equivalent amino acids; and
a substitution of the amino acid that occupies position 359 with a different amino acid selected from S, L and equivalent amino acids.

In further related embodiments, the polypeptide has four alterations relative to SEQ ID NO:11 selected from:
a substitution of the amino acid that occupies position 83 with a different amino acid selected from A and equivalent amino acids;
a substitution of the amino acid that occupies position 169 with a different amino acid selected from S, G, H, D and equivalent amino acids;
a substitution of the amino acid that occupies position 186 with a different amino acid selected from C, M and equivalent amino acids; and
a substitution of the amino acid that occupies position 359 with a different amino acid selected from S, L and equivalent amino acids.

In further related embodiments, the polypeptide has five alterations relative to SEQ ID NO:11 selected from:
a substitution of the amino acid that occupies position 83 with a different amino acid selected from A and equivalent amino acids;
a substitution of the amino acid that occupies position 169 with a different amino acid selected from S, G, H, D and equivalent amino acids;
a substitution of the amino acid that occupies position 186 with a different amino acid selected from C, M and equivalent amino acids; and
a substitution of the amino acid that occupies position 359 with a different amino acid selected from S, L and equivalent amino acids.

In further related embodiments, the polypeptide has six alterations relative to SEQ ID NO:11 selected from:
a substitution of the amino acid that occupies position 83 with a different amino acid selected from A and equivalent amino acids;
a substitution of the amino acid that occupies position 169 with a different amino acid selected from S, G, H, D and equivalent amino acids;
a substitution of the amino acid that occupies position 186 with a different amino acid selected from C, M and equivalent amino acids; and
a substitution of the amino acid that occupies position 359 with a different amino acid selected from S, L and equivalent amino acids.

In further related embodiments, the polypeptide has seven alterations relative to SEQ ID NO:11 selected from:
a substitution of the amino acid that occupies position 83 with a different amino acid selected from A and equivalent amino acids;
a substitution of the amino acid that occupies position 169 with a different amino acid selected from S, G, H, D and equivalent amino acids;
a substitution of the amino acid that occupies position 186 with a different amino acid selected from C, M and equivalent amino acids; and
a substitution of the amino acid that occupies position 359 with a different amino acid selected from S, L and equivalent amino acids.

In further related embodiments, the polypeptide has eight alterations relative to SEQ ID NO:11 selected from:
a substitution of the amino acid that occupies position 83 with a different amino acid selected from A and equivalent amino acids;
a substitution of the amino acid that occupies position 169 with a different amino acid selected from S, G, H, D and equivalent amino acids;
a substitution of the amino acid that occupies position 186 with a different amino acid selected from C, M and equivalent amino acids; and
a substitution of the amino acid that occupies position 359 with a different amino acid selected from S, L and equivalent amino acids.

In further related embodiments, the polypeptide has nine alterations relative to SEQ ID NO:11 selected from:
a substitution of the amino acid that occupies position 83 with a different amino acid selected from A and equivalent amino acids;
a substitution of the amino acid that occupies position 169 with a different amino acid selected from S, G, H, D and equivalent amino acids;
a substitution of the amino acid that occupies position 186 with a different amino acid selected from C, M and equivalent amino acids; and
a substitution of the amino acid that occupies position 359 with a different amino acid selected from S, L and equivalent amino acids.

Another embodiment provides a polypeptide that has only one of these nine specified substitutions relative to SEQ ID NO:11. In one embodiment, the substitution is R169H. In one embodiment, the substitution is R169D. In one embodiment, the substitution is I186M. In one embodiment, the substitution is R359S. In one embodiment, any of these polypeptides has improved specific activity in the catalysis of the dehydration of linalool to myrcene.

Another embodiment provides a polypeptide that has only two of these nine specified substitutions relative to SEQ ID NO:11. In one embodiment, the two substitutions are H83A and R169S. In one embodiment, the two substitutions are H83A and R169G. In one embodiment, the two substitutions are H83A and I186C. In one embodiment, the two substitutions are H83A and R359S. In one embodiment, the two substitutions are H83A and R359L. In one embodiment, any of these polypeptides has improved specific activity in the catalysis of the dehydration of linalool to myrcene.

Another embodiment provides a polypeptide that has only three of these nine specified substitutions relative to SEQ ID NO:11. In one embodiment, any of these polypeptides has improved specific activity in the catalysis of the dehydration of linalool to myrcene.

Another embodiment provides a polypeptide that has only four of these nine specified substitutions relative to SEQ ID NO:11. In one embodiment, any of these polypeptides has improved specific activity in the catalysis of the dehydration of linalool to myrcene.

Another embodiment provides a polypeptide that has only five of these nine specified substitutions relative to SEQ ID NO:11. In one embodiment, any of these polypeptides has improved specific activity in the catalysis of the dehydration of linalool to myrcene.

Another embodiment provides a polypeptide that has only six of these nine specified substitutions relative to SEQ ID NO:11. In one embodiment, any of these polypeptides has improved specific activity in the catalysis of the dehydration of linalool to myrcene.

Another embodiment provides a polypeptide that has only seven of these nine specified substitutions relative to SEQ ID NO:11. In one embodiment, any of these polypeptides has improved specific activity in the catalysis of the dehydration of linalool to myrcene.

Another embodiment provides a polypeptide that has only eight of these nine specified substitutions relative to SEQ ID NO:11. In one embodiment, any of these polypeptides has improved specific activity in the catalysis of the dehydration of linalool to myrcene.

Another embodiment provides a polypeptide that has only nine of these nine specified substitutions relative to SEQ ID NO:11. In one embodiment, any of these polypeptides has improved specific activity in the catalysis of the dehydration of linalool to myrcene.

Another embodiment provides a polypeptide according to any one of paragraphs [0199] to [0221], wherein the improved/increased specific activity in the catalysis of the dehydration of linalool to myrcene is observed in at least one type of non-bacterial cells.

Another embodiment provides a polypeptide according to any one of paragraphs [0199] to [0221], wherein the improved/increased specific activity in the catalysis of the dehydration of linalool to myrcene is observed in at least one type of bacteria.

Another embodiment provides a polypeptide according to any one of paragraphs [0199] to [0221], wherein the improved/increased specific activity in the catalysis of the dehydration of linalool to myrcene is observed in more than one type of bacteria. In some embodiments, the bacteria are a strain of E. coli. In some embodiments, the bacteria are Origami2(DE3), BL21(DE3), or a related strain.

Another embodiment provides a polypeptide according to any one of paragraphs [0199] to [0221], wherein the polypeptide has both alterations that improve/increase the specific activity in the catalysis of the dehydration of linalool to myrcene, and/or alterations that improve solubility, and/or alterations that improve specific activity in the catalysis of the dehydration of 3-buten-2-ol to 1,3-butadiene

Another embodiment provides a polypeptide according to any one of paragraphs [0199] to [0221], wherein one or more additional substitutions, deletions, insertions, and/or inversions are introduced into the polypeptide.

Another embodiment provides a polypeptide according to any one of paragraphs [0199] to [0221], wherein the polypeptide further contains an N-terminal periplasmic tag.

Another embodiment provides a polypeptide according to any one of paragraphs [0199] to [0221], wherein the polypeptide lacks an N-terminal periplasmic tag.

Another embodiment provides a polypeptide according to any one of paragraphs [0199] to [0221], wherein the polypeptide further contains an N-terminal periplasmic tag and a C-terminal poly-His tag.

Another embodiment provides a polypeptide according to any one of paragraphs [0199] to [0221], wherein lacks a N-terminal periplasmic tag and contains a C-terminal poly-His tag.

Another embodiment provides a polypeptide according to any one of paragraphs [0199] to [0221], wherein the polypeptide further contains a C-terminal poly-His tag.

Another embodiment provides a composition comprising a substrate and a means for enzymatically producing myrcene from said substrate.

Another embodiment provides a method of producing myrcene comprising: a step for enzymatically converting linalool to myrcene; and measuring and/or harvesting the myrcene thereby produced.

Another embodiment provides an apparatus comprising a container and a means for producing myrcene.

Another embodiment provides a method of designing a polypeptide with improved specific activity in the catalysis of the dehydration of linalool to myrcene relative to that of a polypeptide consisting of SEQ ID NO: 11, 13, 37, OR 38, the method comprising mutating a means for enzymatically converting linalool to myrcene.

It will be understood that additional embodiments encompass polypeptides incorporating both modifications that improve stability, modifications that improve the catalysis of the dehydration of 3-methyl-3-buten-2-ol to isoprene, and modifications that improve specific activity in the catalysis of the dehydration of 3-buten-2-ol to 1,3-butadiene. Furthermore, it may be advantageous to introduce additional point-mutations (e.g., deletions, insertions, inversions, substitutions) in any of the polypeptides described herein.

Any of the polypeptides described herein may either contain or lack a N-terminal periplasmic tag. In some embodiments, the periplasmic tag is that sequence underlined in the protein of SEQ ID NO. 11. In one embodiment, the polypeptide may contain a C-terminal tag. In some embodiments, the C-terminal tag is a poly-Histidine tag consisting of six Histidines. In some embodiments, the polypeptide contains both a periplasmic tag and a C-terminal tag. In some embodiments, the polypeptide contains only a periplasmic tag. In some embodiments, the polypeptide contains a C-terminal tag. In any of these embodiments, the C-terminal tag can be a poly-Histidine tag.

In one embodiment, the amino acid sequence of the polypeptide is that of SEQ ID NO. 14. In one embodiment, the amino acid sequence of the polypeptide is that of SEQ ID NO. 15. In one embodiment, the amino acid sequence of the polypeptide is that of SEQ ID NO. 25. In one embodiment, the amino acid sequence of the polypeptide is that of SEQ ID NO. 32. In one embodiment, the amino acid sequence of the polypeptide is that of SEQ ID NO. 35. In one embodiment, the amino acid sequence of the polypeptide is that of SEQ ID NO. 36. All of these polypeptides lack periplasmic tags but have poly-His tags.

In one embodiment, the amino acid sequence of the polypeptide is that of SEQ ID NO. 14 without the poly-His tag. In one embodiment, the amino acid sequence of the polypeptide is that of SEQ ID NO. 15 without the poly-His tag. In one embodiment, the amino acid sequence of the polypeptide is that of SEQ ID NO. 25 without the poly-His tag. In one embodiment, the amino acid sequence of the polypeptide is that of SEQ ID NO. 32 without the poly-His tag. In one embodiment, the amino acid sequence of the polypeptide is that of SEQ ID NO. 35 without the poly-His tag. In one embodiment, the amino acid sequence of the polypeptide is that of SEQ ID NO. 36 without the poly-His tag.

Improved Activity in the Catalysis of the Dehydration of 3-methyl-3-buten-2-ol to Isoprene.

Provided herein are also polypeptides with improved activity in the catalysis of the dehydration of 3-methyl-3-buten-2-ol to isoprene; compositions comprising such polypeptides; nucleic acids encoding them, host cells comprising such nucleic acids, antibodies against such polypeptides, and a variety of methods of making and using such polypeptides. Compositions comprising a substrate and a means for producing isoprene are also provided. These are described in more detail in the EXAMPLES, claims, and SUMMARY sections of this disclosure.

Derivatives of the mutant polypeptides are also provided.

In one embodiment, derivative polypeptides are polypeptides that have been further altered, for example by conjugation or complexing with other chemical moieties, by post-translational modification (e.g. phosphorylation, acetylation and the like), modification of glycosylation (e.g. adding, removing or altering glycosylation), and/or inclusion/substitution of additional amino acid sequences as would be understood in the art.

Additional amino acid sequences may include fusion partner amino acid sequences which create a fusion protein. By way of example, fusion partner amino acid sequences may assist in detection and/or purification of the isolated fusion protein. Non-limiting examples include metal-binding (e.g. poly-histidine) fusion partners, maltose binding protein (MBP), Protein A, glutathione S-transferase (GST), fluorescent protein sequences (e.g. GFP), epitope tags such as myc, FLAG, and haemagglutinin tags.

Other derivatives contemplated by the embodiments include, but are not limited to, modification to side chains, incorporation of unnatural amino acids and/or their derivatives during peptide, or protein synthesis and the use of crosslinkers and other methods which impose conformational constraints on the polypeptides and fragments thereof.

Nucleic Acids

The embodiments also encompass nucleic acid molecules encoding relatives of the polypeptides disclosed herein. “Relatives” of the polypeptide-encoding nucleic acid sequences include those sequences that encode the polypeptides disclosed herein but that differ conservatively because of the degeneracy of the genetic code. Allelic variants that later develop through culture can be identified with the use of well-known molecular biology techniques, such as polymerase chain reaction (PCR) and hybridization techniques as outlined below. Relative nucleic acid sequences also include synthetically derived nucleic acid sequences that have been generated, for example, by using site-directed mutagenesis but which still encode the polypeptides disclosed.

The skilled artisan will further appreciate that changes can be introduced by mutation of the nucleic acid sequences thereby leading to changes in the amino acid sequence of the encoded polypeptides, without altering the biological activity of these proteins. Thus, relative nucleic acid molecules can be created by introducing one or more nucleotide substitutions, nucleotide additions and/or nucleotide deletions into the corresponding nucleic acid sequence disclosed herein, such that one or more amino acid substitutions, amino acid additions or amino acid deletions are introduced into the encoded protein. Mutations can be introduced by standard techniques, such as site-directed mutagenesis and PCR-mediated mutagenesis. Such relative nucleic acid sequences are also encompassed by the present embodiments.

Alternatively, variant nucleic acid sequences can be made by introducing mutations randomly along all or part of the coding sequence, such as by saturation mutagenesis and the resultant mutants can be screened for ability to confer improved solubility or increased specific activity in the catalysis of the dehydration of 3-buten-2-ol to 1,3-butadiene to identify mutants that retain the improved activity of the polypeptides described herein. Following mutagenesis, the encoded protein can be expressed recombinantly, and the activity of the protein can be determined using standard assay techniques, including those described herein.

With the polypeptides and their aminoacid sequence as disclosed herein, the skilled person may determine suitable polynucleotides that encode those polypeptides. Those having ordinary skill in the art will readily appreciate that due to the degeneracy of the genetic code, a multitude of nucleotide sequences encoding the polypeptides described herein exist. The sequence of the polynucleotide gene can be deduced from a polypeptide sequence through use of the genetic code. Computer programs such as “BackTranslate” (GCG™ Package, Acclerys, Inc. San Diego, Calif.) can be used to convert a peptide sequence to the corresponding nucleotide sequence encoding the peptide. Furthermore, synthetic polypeptide polynucleotide sequences as described herein can be designed so that they will be expressed in any cell type, prokaryotic or eukaryotic.

Accordingly, some embodiments relate to polynucleotides either comprising or consisting essentially of a nucleic acid sequence encoding a polypeptide as described above and elsewhere herein. In some embodiments, the nucleic acid sequence is a DNA sequence (e.g., a cDNA sequence). In other embodiments, the nucleic acid sequence is a RNA sequence. In some embodiments, the nucleic acid is a cDNA encoding any of the polypeptides described herein. The nucleotide sequences encoding the polypeptides may be prepared by any suitable technologies well known to those skilled in the art, including, but not limited to, recombinant DNA technology and chemical synthesis. Synthetic polynucleotides may be prepared using commercially available automated polynucleotide synthesizers.

One aspect pertains to isolated or recombinant nucleic acid molecules comprising nucleic acid sequences encoding the polypeptides described herein or biologically active portions thereof, as well as nucleic acid molecules sufficient for use as hybridization probes to identify nucleic acid molecules encoding proteins with regions of sequence homology to the polypeptides described herein. Nucleic acid molecules that are fragments of these nucleic acid sequences encoding polypeptides are also encompassed by the embodiments. By “fragment” is intended a portion of the nucleic acid sequence encoding a portion of a polypeptide. In some embodiments, a fragment of a nucleic acid sequence may encode a biologically active portion of a polypeptide or it may be a fragment that can be used as a hybridization probe or PCR primer using methods well known to one of ordinary skill in the art.

In some embodiments, the nucleic acid has been codon optimized for expression of any one of the polypeptides described herein.

In other embodiments, the nucleic acid is a probe, which may be a single or double-stranded oligonucleotide or polynucleotide, suitably labeled for the purpose of detecting complementary sequences of polynucleotides encoding the variants described herein, such as in arrays, Northern, or Southern blotting. Methods for detecting labeled nucleic acids hybridized to an immobilized nucleic acid are well known to practitioners in the art. Such methods include autoradiography, chemiluminescent, fluorescent and colorimetric detection.

In some embodiments, the polynucleotide comprises a sequence encoding any one of the polypeptides described herein operably linked to a promoter sequence. Constitutive or inducible promoters as known in the art are contemplated herein. The promoters may be either naturally occurring promoters, or hybrid promoters that combine elements of more than one promoter. Non-limiting examples of promoters include SV40, cytomegalovirus (CMV), and HIV-1 LTR promoters.

In some embodiments, the polynucleotide comprises a sequence encoding any one of the polypeptides described herein operably linked to a sequence encoding another protein, which can be a fusion protein or another protein separated by a linker. In some embodiments, the linker has a protease cleavage site, such as for Factor Xa or Thrombin, which allow the relevant protease to partially digest the fusion variant polypeptide described herein and thereby liberate the recombinant polypeptide therefrom. The liberated polypeptide can then be isolated from the fusion partner by, for example, subsequent chromatographic separation. In some embodiments, the polynucleotide comprises a sequence encoding any one of the polypeptides described herein operably linked to both a promoter and a fusion protein.

Some other embodiments provide genetic constructs in the form of, or comprising genetic components of, a plasmid, bacteriophage, a cosmid, a yeast or bacterial artificial chromosome, as are well understood in the art. Genetic constructs may be suitable for maintenance and propagation of the isolated nucleic acid in bacteria or other host cells, for manipulation by recombinant DNA technology and/or expression (expression vectors) of the nucleic acid or an encoded polypeptide as described herein.

Some other embodiments relate to recombinant expression vectors comprising a DNA sequence encoding one or more of the polypeptides described herein. In some embodiments, the expression vector comprises one or more of said DNA sequences operably linked to a promoter. Suitably, the expression vector comprises the nucleic acid encoding one of the polypeptides described herein operably linked to one or more additional sequences. In some embodiments, the expression vector may be either a self-replicating extra-chromosomal vector such as a plasmid, or a vector that integrates into a host genome. Non-limiting examples of viral expression vectors include adenovirus vectors, adeno-associated virus vectors, herpes viral vectors, retroviral vectors, lentiviral vectors, and the like. For example, adenovirus vectors can be first, second, third, and/or fourth generation adenoviral vectors or gutless adenoviral vectors. Adenovirus vectors can be generated to very high titers of infectious particles, infect a great variety of cells, efficiently transfer genes to cells that are not dividing, and are seldom integrated in the host genome, which avoids the risk of cellular transformation by insertional mutagenesis. The vector may further include sequences flanking the polynucleotide giving rise to RNA which comprise sequences homologous to eukaryotic genomic sequences or viral genomic sequences. This will allow the introduction of the polynucleotides described herein into the genome of a host cell.

An integrative cloning vector may integrate at random or at a predetermined target locus in the chromosome(s) of the host cell into which it is to be integrated.

Specific embodiments of expression vectors can be found elsewhere in this disclosure (see below).

Some other embodiments relate to host cells comprising a DNA molecule encoding a polypeptide as described herein. In some embodiments, these host cells can be described as expression systems. Suitable host cells for expression may be prokaryotic or eukaryotic. Without limitation, suitable host cells may be mammalian cells (e.g. HeLa, HEK293T, Jurkat cells), yeast cells (e.g. Saccharomyces cerevisiae), insect cells (e.g. Sf9, Trichoplusia ni) utilized with or without a baculovirus expression system, or bacterial cells, such as E. coli (Origami2(DE3), BL21(DE3)), or a Vaccinia virus host. Introduction of genetic constructs into host cells (whether prokaryotic or eukaryotic) is well known in the art, as for example described in Current Protocols in Molecular Biology Eds. Ausubel et al., (John Wiley & Sons, Inc. current update Jul. 2, 2014).

A further embodiment relates to a transformed or transduced organism, such as an organism selected from plant and insect cells, bacteria, yeast, baculovirus, protozoa, nematodes, algae, and transgenic mammals (mice, rats, pigs, etc.). The transformed organism comprises a DNA molecule of the embodiments, an expression cassette comprising the DNA molecule or a vector comprising the expression cassette, which may be stably incorporated into the genome of the transformed organism.

Methods for Preparing the Disclosed Polypeptides

The polypeptides described herein (inclusive of fragments and derivatives) may be prepared by any suitable procedure known to those of skill in the art. In some embodiments, the protein is a recombinant protein.

By way of example only, a recombinant polypeptide may be produced by a method including the steps of: (i) preparing an expression construct which comprises a nucleic acid expressing one or more of the polypeptides described herein, operably linked to one or more regulatory nucleotide sequences; (ii) transfecting or transforming a suitable host cell with the expression construct; (iii) expressing a recombinant protein in said host cell; and (iv) isolating the recombinant protein from said host cell or using the resultant host cell as is or as a cell extract.

Several methods for introducing mutations into genes, cDNA, and other polynucleotides are known in the art, including the use of proprietary library generation methods that are commercially available. The DNA sequence encoding a wild-type LDH may be isolated from any cell or microorganism producing the LDH in question, using various methods well known in the art. In one embodiment, the cDNA encoding the wild-type LDH is obtained from Castellaniella defragrans cells, cDNA libraries, or the like.

In one embodiment, the mutations are introduced into a wild-type LDH using Site-Directed Mutagenesis. Once an LDH-encoding DNA sequence has been isolated, and desirable sites for mutation identified, mutations may be introduced using synthetic oligonucleotides. These oligonucleotides contain nucleotide sequences flanking the desired mutation sites; mutant nucleotides are inserted during oligonucleotide synthesis. In a specific method, a single-stranded gap of DNA, bridging the LDH-encoding sequence, is created in a vector carrying the LDH gene. Then the synthetic nucleotide, bearing the desired mutation, is annealed to a homologous portion of the single-stranded DNA. The remaining gap is then filled in with DNA polymerase I (Klenow fragment) and the construct is ligated using T4 ligase.

Another embodiment for introducing mutations into LDH-encoding DNA sequences involves the 3-step generation of a PCR fragment containing the desired mutation introduced by using a chemically synthesized DNA strand as one of the primers in the PCR reactions. From the PCR-generated fragment, a DNA fragment carrying the mutation may be isolated by cleavage with restriction endonucleases and reinserted into an expression plasmid.

Expression of the Polypeptides

According to some embodiments, a DNA sequence encoding the polypeptide produced by methods described above, or by any alternative methods known in the art, can be expressed, in enzyme form, using an expression vector, which typically includes control sequences encoding a promoter, operator, ribosome binding site, translation initiation signal, and, optionally, a repressor gene or various activator genes. For each combination of a promoter and a host cell, culture conditions are available which are conducive to the expression the DNA sequence encoding the desired polypeptide. After reaching the desired cell density or titre of the polypeptide the culture is stopped and the polypeptide is recovered using known procedures. Alternatively, the host cell is used directly (e.g., pellet, suspension), i.e., without isolation of the recombinant protein.

The recombinant expression vector carrying the DNA sequence encoding a polypeptide as described herein may be any vector, which may conveniently be subjected to recombinant DNA procedures, and the choice of vector will often depend on the host cell into which it is to be introduced. Thus, the vector may be an autonomously replicating vector, i.e., a vector that exists as an extrachromosomal entity, the replication of which is independent of chromosomal replication, e.g., a plasmid, a bacteriophage or an extrachromosomal element, minichromosome, or an artificial chromosome. Alternatively, the vector may be one which, when introduced into a host cell, is integrated into the host cell genome and replicated together with the chromosome(s) into which it has been integrated.

In the vector, the DNA sequence typically is operably connected to a suitable promoter sequence. The promoter may be any DNA sequence that shows transcriptional activity in the host cell of choice and may be derived from genes encoding proteins either homologous or heterologous to the host cell. Examples of suitable promoters for directing the transcription of the DNA sequence encoding a polypeptide as described herein, especially in a bacterial host, are the promoter of the lac operon of E. coli, the Streptomyces coelicolor agarase gene dagA promoters, the promoters of the Castellaniella defragrans, and others. For transcription in a fungal host, examples of useful promoters are those derived from the gene encoding A. oryzae TAKA amylase, Rhizomucor miehei aspartic proteinase, A. niger neutral LDH, A. niger acid stable LDH, A. niger glucoamylase, Rhizomucor miehei lipase, A. oryzae alkaline protease, A. oryzae triose phosphate isomerase or A. nidulans acetamidase. The promoters can be selected based on the desired outcome. The nucleic acids can be combined with constitutive, tissue-preferred, inducible, or other promoters for expression in the host cell or organism. The above list of promoters is not meant to be limiting. Any appropriate promoter can be used in the embodiments.

In some embodiments, the expression vector described may also comprise a suitable transcription terminator and, in eukaryotes, polyadenylation sequences operably connected to the DNA sequence encoding the polypeptide as described herein. Termination and polyadenylation sequences may suitably be derived from the same sources as the promoter or not.

In some embodiments, the vector may further comprise a DNA sequence enabling the vector to replicate in the host cell in question. Examples of such sequences are the origins of replication of plasmids pUC19, pACYC177, pUB110, pE194, pAMB1 and pIJ702. The above list of origins of replication is not meant to be limiting. Any appropriate origins of replication can be used in the embodiments

In some embodiments, the vector may also comprise a selectable marker. Selectable marker genes are utilized for the selection of transformed cells or tissues, e.g., a gene the product of which complements a defect in the host cell, such as the dal genes from B. subtilis or B. licheniformis, or one which confers antibiotic resistance such as ampicillin, kanamycin, chloramphenicol or tetracyclin resistance. Furthermore, the vector may comprise Aspergillus selection markers such as amdS, argB, niaD and sC, a marker giving rise to hygromycin resistance, or the selection may be accomplished by co-transformation. The above list of selectable marker genes is not meant to be limiting. Any selectable marker gene can be used in the embodiments.

Appropriate culture media and conditions for the above-described host cells are known in the art. While intracellular expression may be advantageous in some respects, e.g., when using certain bacteria as host cells, it is often preferred that the expression is extracellular or periplasmic. In some embodiments, the Castellaniella defragrans LDHs mentioned herein comprise a pre-region/signal/leader sequence permitting secretion of the expressed protease into the culture medium or periplasm. If desirable, this pre-region may be replaced by a different preregion or signal sequence, conveniently accomplished by substitution of the DNA sequences encoding the respective preregions.

The procedures used to ligate the DNA construct encoding a polypeptide, the promoter, terminator and other elements, respectively, and to insert them into suitable vectors containing the information necessary for replication, are well known to persons skilled in the art (cf., for instance, Sambrook et al., Molecular Cloning: A Laboratory Manual, supra).

The cells disclosed herein, either comprising a DNA construct or an expression vector as defined above, are advantageously used as host cells in the recombinant production of a polypeptide as described herein. The cell may be transformed with the DNA construct encoding the polypeptide as described herein, conveniently by integrating the DNA construct (in one or more copies) in the host chromosome. This integration is generally considered to be an advantage as the DNA sequence is more likely to be stably maintained in the cell. Integration of the DNA constructs into the host chromosome may be performed according to conventional methods, e.g., by homologous or heterologous recombination. Alternatively, the cell may be transformed with an expression vector as described above in connection with the different types of host cells.

In some embodiments, a cell as described herein may be a cell of a higher organism such as a mammal or an insect, a microbial cell, e.g., a bacterial or a fungal (including yeast) cell, or the like.

Without limitation, examples of suitable bacteria are Castellaniella defragrans, gram-positive bacteria such as Bacillus subtilis, Bacillus licheniformis, Bacillus lentus, Bacillus brevis, Bacillus stearothermophilus, Bacillus alkalophilus, Bacillus amyloliquefaciens, Bacillus coagulans, Bacillus circulans, Bacillus lautus, Bacillus megaterium, Bacillus thuringiensis, or Streptomyces lividans or Streptomyces murinus, or gram negative bacteria such as E. coli. In one embodiment, the transformation of the bacteria may, for instance, be effected by protoplast transformation or by using competent cells in a manner known per se.

In some other embodiments, a yeast organism may be selected from a species of Saccharomyces or Schizosaccharomyces, e.g., Saccharomyces cerevisiae. The filamentous fungus may advantageously belong to a species of Aspergillus, e.g., Aspergillus oryzae or Aspergillus niger. Fungal cells may be transformed by a process involving protoplast formation and transformation of the protoplasts followed by regeneration of the cell wall in a manner known per se. Suitable procedure for transformation fungal host cells are well known in the art.

In yet a further set of embodiments, the present disclosure relates to a method of producing a polypeptide as described herein, which method comprises cultivating a host cell as described above under conditions conducive to the production of the variant and recovering the variant from the cells and/or culture medium. In some embodiments, the cells are cultured under aerobic conditions. In other embodiments, the cells are cultured under anaerobic conditions.

The medium used to cultivate the cells may be any conventional medium suitable for growing the host cell in question and obtaining expression of the polypeptide as described herein. Suitable media are available from commercial suppliers or may be prepared according to published recipes (e.g., as described in catalogues of the American Type Culture Collection).

Purification of the Polypeptides

The polypeptide secreted from the host cells may conveniently be recovered from the culture medium by well-known procedures, including separating the cells from the medium by centrifugation or filtration, and precipitating proteinaceous components of the medium by means of a salt such as ammonium sulphate, followed by the use of chromatographic procedures such as ion exchange chromatography, affinity chromatography (e.g., Ni—Cd), or the like.

For example, fermentation, separation, and concentration techniques are known in the art and conventional methods can be used in order to prepare the concentrated polypeptide-containing solution. After fermentation, a fermentation broth is obtained, and the microbial cells and various suspended solids, including residual raw fermentation materials, are removed by conventional separation techniques to obtain a polypeptide solution. Filtration, centrifugation, microfiltration, rotary vacuum drum filtration, followed by ultra-filtration, extraction or chromatography, or the like are generally used.

In some instances, it is desirable to concentrate the solution containing the polypeptide to optimize recovery, since the use of unconcentrated solutions requires increased incubation time to collect concentrates containing the purified polypeptide. The solution is concentrated using conventional techniques until the desired enzyme concentration is obtained. Concentration of the enzyme variant containing solution may be achieved by any of the techniques discussed above. In one embodiment, rotary vacuum evaporation and/or ultrafiltration is used.

In one embodiment, a “precipitation agent” for purposes of purification is meant to be a compound effective to precipitate the polypeptide from the concentrated enzyme variant solution in solid form, whatever its nature may be, i.e., crystalline, amorphous, or a blend of both. Precipitation can be performed using, for example, a metal halide precipitation agent. Metal halide precipitation agents include: alkali metal chlorides, alkali metal bromides and blends of two or more of these metal halides. The metal halide may be selected from the group consisting of sodium chloride, potassium chloride, sodium bromide, potassium bromide and blends of two or more of these metal halides. Suitable metal halides include sodium chloride and potassium chloride, particularly sodium chloride, which can further be used as a preservative.

In one embodiment, a metal halide precipitation agent is used in an amount effective to precipitate the polypeptide. The selection of at least an effective amount and an optimum amount of metal halide effective to cause precipitation of the enzyme variant, as well as the conditions of the precipitation for maximum recovery including incubation time, pH, temperature and concentration of polypeptide, will be readily apparent to one of ordinary skill in the art after routine testing.

In some embodiments, at least about 5% w/v (weight/volume) to about 25% w/v of metal halide is added to the concentrated enzyme polypeptide solution, and usually at least 8% w/v. In some embodiments, no more than about 25% w/v of metal halide is added to the concentrated enzyme polypeptide solution and usually no more than about 20% w/v. The optimal concentration of the metal halide precipitation agent will depend, among others, on the nature of the specific polypeptide and on its concentration in the concentrated polypeptide solution.

Another alternative embodiment to effect precipitation of the enzyme is to use of organic compounds, which can be added to the concentrated enzyme polypeptide solution. The organic compound precipitating agent can include: 4-hydroxybenzoic acid, alkali metal salts of 4-hydroxybenzoic acid, alkyl esters of 4-hydroxybenzoic acid, and blends of two or more of these organic compounds. The addition of the organic compound precipitation agents can take place prior to, simultaneously with or subsequent to the addition of the metal halide precipitation agent, and the addition of both precipitation agents, organic compound and metal halide, may be carried out sequentially or simultaneously.

In some embodiments, the organic compound precipitation agents are selected from the group consisting of alkali metal salts of 4-hydroxybenzoic acid, such as sodium or potassium salts, and linear or branched alkyl esters of 4-hydroxybenzoic acid, wherein the alkyl group contains from 1 to 12 carbon atoms, and blends of two or more of these organic compounds. In some embodiments, the organic compound precipitations agents can be for example linear or branched alkyl esters of 4-hydroxybenzoic acid, wherein the alkyl group contains from 1 to 10 carbon atoms, and blends of two or more of these organic compounds. In some embodiments, suitable organic compounds include linear alkyl esters of 4-hydroxybenzoic acid, wherein the alkyl group contains from 1 to 6 carbon atoms, and blends of two or more of these organic compounds. Methyl esters of 4-hydroxybenzoic acid, propyl ester of 4-hydroxybenzoic acid, butyl ester of 4-hydroxybenzoic acid, ethyl ester of 4-hydroxybenzoic acid and blends of two or more of these organic compounds can also be used. Additional organic compounds also include, but are not limited to, 4-hydroxybenzoic acid methyl ester (methyl PARABEN) and 4-hydroxybenzoic acid propyl ester (propyl PARABEN), which are also amylase preservative agents.

In some embodiments, addition of the organic compound precipitation agent provides the advantage of high flexibility of the precipitation conditions with respect to pH, temperature, polypeptide concentration, precipitation agent concentration, and time of incubation.

In some embodiments, the organic compound precipitation agent is used in an amount effective to improve precipitation of the enzyme polypeptide by means of the metal halide precipitation agent. The selection of at least an effective amount and an optimum amount of organic compound precipitation agent, as well as the conditions of the precipitation for maximum recovery including incubation time, pH, temperature and concentration of enzyme variant, will be readily apparent to one of ordinary skill in the art, in light of the present disclosure, after routine testing.

In some embodiments, at least about 0.01% w/v of organic compound precipitation agent is added to the concentrated enzyme polypeptide solution and usually at least about 0.02% w/v. In some embodiments, no more than about 0.3% w/v of organic compound precipitation agent is added to the concentrated enzyme polypeptide solution and usually no more than about 0.2% w/v.

In some embodiments, the concentrated enzyme polypeptide solution, containing the metal halide precipitation agent and, in one aspect, the organic compound precipitation agent, is adjusted to a pH that necessarily will depend on the enzyme variant to be purified. In some embodiments, the pH is adjusted to a level near the isoelectric point (pI) of the polypeptide. For example, the pH can be adjusted within a range of about 2.5 pH units below the pI to about 2.5 pH units above the pI.

The incubation time necessary to obtain a purified enzyme polypeptide precipitate depends on the nature of the specific enzyme polypeptide, the concentration of polypeptide, and the specific precipitation agent(s) and its (their) concentration. In some embodiments, the time effective to precipitate the enzyme polypeptide is between about 1 to about 30 hours; usually it does not exceed about 25 hours. In the presence of the organic compound precipitation agent, the time of incubation can still be reduced to less than about 10 hours, and in most cases even about 6 hours.

In some embodiments, the temperature during incubation is between about 4° C. and about 50° C. In some embodiments, the method is carried out at a temperature between about 10° C. and about 45° C., and particularly between about 20° C. and about 40° C. The optimal temperature for inducing precipitation varies according to the solution conditions and the enzyme variant or precipitation agent(s) used.

In some embodiments, the overall recovery of purified enzyme polypeptide precipitate, and the efficiency with which the process is conducted, is improved by agitating the solution comprising the enzyme polypeptide, the added metal halide and the added organic compound. In some embodiments, the agitation step is done both during addition of the metal halide and the organic compound, and during the subsequent incubation period. Suitable agitation methods include mechanical stirring or shaking, vigorous aeration, or any similar technique.

In some embodiments, after the incubation period, the purified enzyme polypeptide is then separated from the impurities and collected by conventional separation techniques, such as filtration, centrifugation, microfiltration, rotary vacuum filtration, ultrafiltration, press filtration, cross membrane microfiltration, cross flow membrane microfiltration or the like. Cross membrane microfiltration can be one method used. In some embodiments, further purification of the purified enzyme polypeptide precipitate can be obtained by washing the precipitate with water. For example, the purified enzyme polypeptide precipitate is washed with water containing the metal halide precipitation agent, for example, with water containing the metal halide and the organic compound precipitation agents.

Compositions

Some embodiments relate to compositions comprising one or more of the polypeptides described herein alone or in combination, including in combination with wild type LDH. In some embodiments, the composition comprises one or more polypeptides with improved solubility. In some embodiments, the composition comprises one or more polypeptides with improved increased specific activity in the catalysis of the dehydration of 3-buten-2-ol to 1,3-butadiene. In other embodiments, the composition comprises one or more polypeptides with improved solubility and one or more polypeptides with increased specific activity in the catalysis of the dehydration of 3-buten-2-ol to 1,3-butadiene.

In some embodiments the composition may be composed of polypeptides, from (1) commercial suppliers; (2) cloned genes expressing polypeptides; (3) complex broth (such as that resulting from growth of a microbial strain or any other host cell in media), wherein the strains/host cells secrete polypeptides into the media; (4) cell lysates of strains/host cells grown as in (3); and/or (5) any other host cell material expressing polypeptides. Different polypeptides in a composition may be obtained from different sources.

In some embodiments, the composition comprises 3-buten-2-ol and one or more polypeptides described herein. In other embodiments, the composition further comprises a wild-type LDH.

In some embodiments, the composition comprises 1,3-butadiene and one or more polypeptide described herein. In other embodiments, the composition further comprises a wild-type LDH.

In some embodiments, the composition comprises a rubber product polymerized from 1,3-butadiene produced in the presence of a polypeptide as described herein.

In some embodiments, the composition comprises a copolymer polymerized from 1,3-butadiene produced in the presence of a polypeptide as described herein.

In some embodiments, the composition comprises a plastic product polymerized from 1,3-butadiene produced in the presence of a polypeptide as described herein.

Antibodies capable of binding to a polypeptide of the embodiments, or to relatives or fragments thereof that encompass at least one of the improved mutations/alterations described herein, are also encompassed. Methods for producing antibodies are well known in the art (see, for example, Harlow and Lane, (1988) Antibodies: A Laboratory Manual, Cold Spring Harbor Laboratory, Cold Spring Harbor, N.Y.).

Methods of Use

The polypeptides, nucleic acids, and compositions described herein may be used in many different applications.

One embodiment relates to a method of producing 1,3-butadiene comprising dehydrating 3-buten-2-ol to 1,3-butadiene in the presence of a polypeptide as described herein. Another embodiment related to a method of producing myrcene comprising dehydrating linalool in the presence of a polypeptide as described herein.

Another embodiment relates to the use of the polypeptides described herein in the terpene industry. One embodiment provides for the use of myrcene in the perfume industry, for example, as an intermediate in the production of fragrances. Another embodiment provides myrcene for use in the pharmacological industry. In one embodiment, myrcene produced with any one of the polypeptides described herein can be used as an analgesic. In one embodiment, myrcene produced with any one of the polypeptides described herein can be used as an anti-inflammatory. In one embodiment, myrcene produced with any one of the polypeptides described herein can be used as a sedative.

Another embodiment relates to the use of a polypeptide as described herein in the preparation of a product, wherein the product is polymerized from 1,3-butadiene produced in the presence of the polypeptide. In one embodiment, the product is a rubber product. In one embodiment, the product is a copolymer. In another embodiment, the product is a plastic.

Another embodiment relates to a method of constructing a variant of a wild type LDH of SEQ ID NO:11 or SEQ ID NO:10, which method comprises (a) making alterations in the amino acid sequence each of which is an insertion, a deletion or a substitution of an amino acid residue at one or more positions of SEQ ID NO:11, (b) preparing the variant resulting from those alterations, (c) testing the 1,3-butadiene producing activity of the variant, (d) optionally repeating steps a)-c) recursively; and (e) selecting a variant having an improved 1,3-butadiene producing activity as compared to the wild type LDH of SEQ ID NO:10.

All of the claims presented herein are incorporated by reference, in their full extent, into the specification.

### EXAMPLES

All of the compositions and methods disclosed and claimed herein can be made and executed without undue experimentation in light of the present disclosure and the knowledge of one of ordinary skill in the art. In some cases, the compositions and methods of this disclosure have been described in terms of embodiments; however these embodiments are in no way intended to limit the scope of the claims, and it will be apparent to those of skill in the art that variations may be applied to the compositions and/or methods and in the steps or in the sequence of steps of the methods described herein without departing from the concept, spirit and scope of the invention. More specifically, it will be apparent that certain components which are both chemically and physiologically related may be substituted for the components described herein while the same or similar results would be achieved. All such similar substitutes and modifications apparent to those skilled in the art are deemed to be within the spirit, scope and concept of the invention as defined by the appended claims.

### Example 1

Cloning and Expression of C. defragrans LDH and Other Putative LDHs Cloning

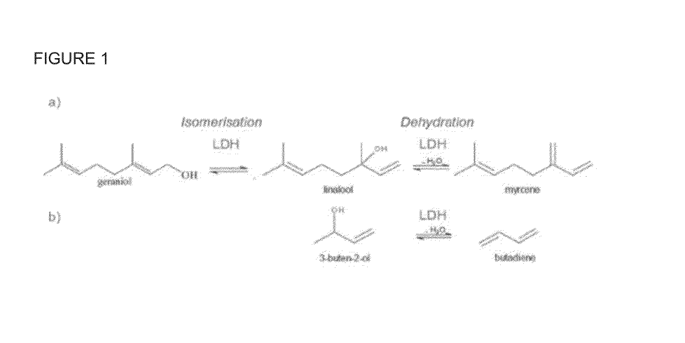

Linalool dehydratase (EC 4.2.1.127) is a unique bi-functional enzyme which naturally catalyzes the dehydration of linalool to myrcene and the isomerization of linalool to geraniol (FIG. 1). Seven different (putative) LDHs were cloned into an expression vector under the control of a T7 promoter (Table 1: for SEQ ID NOs see SEQUENCE LISTING.). The LDH from C. defragrans was provided as a codon-optimized gene. All other genes were ordered as synthetic genes from Geneart/Life Technologies (codon-optimized for E. coli). Based on the LDH from C. defragrans, three different constructs were built: with periplasmic tag (Plasmid-ID: pPI002), without periplasmic tag (Plasmid-ID: pPI003) and without periplasmic tag including a C-terminal His6-tag (SEQ ID NO: 39) (Plasmid-ID: pPI010). All plasmids are listed in Table 1.

TABLE 1
Overview over the initial constructs used in this study.
Plasmids pPI002 to pPI010 are all based on the same
expression plasmid (T7-Promoter, kanamycin-resistance). Table
1 discloses “His₆” as SEQ ID NO: 39.

| Plasmid | | Protein ID | |
|---|---|---|---|
| ID | Gene Abbreviation | (Genbank) | Source |
| pPI001 | LDHCd | E1XUJ2 | C. defragrans |
| (from | (with periplasmic tag | | |
| Invista) | and C-His₆-tag) | | |
| pPI002 | LDHCd | E1XUJ2 | C. defragrans |
| | (with periplasmic tag) | | |
| pPI003 | LDHCd | E1XUJ2 | C. defragrans |
| | (without periplasmic | | |
| | tag) | | |
| pPI004 | LDHCg1 | ELA33010 | Colletotrichum |
| | | | gloeosporioides |
| pPI005 | LDHNp | EOD44468 | Neofusicoccum |
| | | | parvum |
| pPI006 | LDHTI | WP_004338616 | Thauera |
| | | | linaloolentis |
| pPI007 | LDHCg2 | ELA28661 | Colletotrichum |
| | | | gloeosporioides |
| pPI008 | LDHM | YP_004525079 | Mycobacterium |
| | | | sp. JDM601 |
| pPI009 | LDHOt | WP_006561625 | Oscillochloris |
| | | | trichoides |
| pPI010 | LCHCd | E1XUJ2 | C. defragrans |
| | (without periplasmic | | |
| | tag, with C-His₆-tag) | | |

The sequences of all genes used in this study were verified by sequencing and are given in FASTA-format below (see SEQUENCE LISTING).

Growth and Expression

A complex medium (e.g., TB or LB) containing 50 μg/mL kanamycin was inoculated with a fresh overnight culture of the desired strain. Cells were incubated at 37° C. When the culture reached an OD600 of 0.8-1, cells were induced with 0.05 mM IPTG. Expression was overnight at 30° C. After measuring the OD600, the culture was centrifuged and the supernatant discarded. Pellets were used immediately or stored at −20° C.

Improvement in Solubility:

Cell Disruption:

Cells were disrupted by chemo-enzymatic cell lysis using a buffer containing lysozyme, denarase (c-LEcta nuclease, benzonase or DNase can be used as a replace-ment) and detergent based lysis reagent in 50 mM potassium phosphate buffer, pH 7. Cells were suspended to OD600=20 (e.g. a pellet from a culture volume of 2 mL with OD600=12 would be suspended 1.2 mL of lysis buffer).

Cell debris was separated by centrifugation and the supernatant (=soluble fraction) was transferred to fresh tubes. The remaining pellet was suspended in 50 mM potassium phosphate buffer pH 7 in the same volume as the supernatant

Preparation of SDS-Samples:

25 μL supernatant or suspended pellet were mixed with 25 μL 2×SDS-staining reagent and incubated for 5 min at 95° C.

SDS-Page:

10 μL of each sample (containing the soluble or insoluble fraction corresponding to an expression culture with OD600=10) were loaded on a 15% SDS-page. Soluble expression was quantified using the software Gelanalyzer2010.

### Example 2

### Conversion of 3-buten-2-ol to 1,3-butadiene with Whole Cells

To assess the butadiene formation from 3-buten-2-ol, expression cultures were centrifuged (3,275×g, 20 min, and 4° C.) and the pellet suspended in M9-medium (see Example 4) to a cell density of 160 mg/m L. In a 1.5 mL GC glass vial, 1.4 mL of cell suspension were added to 100 μL of a 300 mM 3-buten-2-ol solution (cfinal=20 mM). The vials were sealed immediately and incubated at 30° C., 120 rpm. After the specified time intervals, samples were analyzed for 1,3-butadiene formation by headspace GC, as described in Example 4.

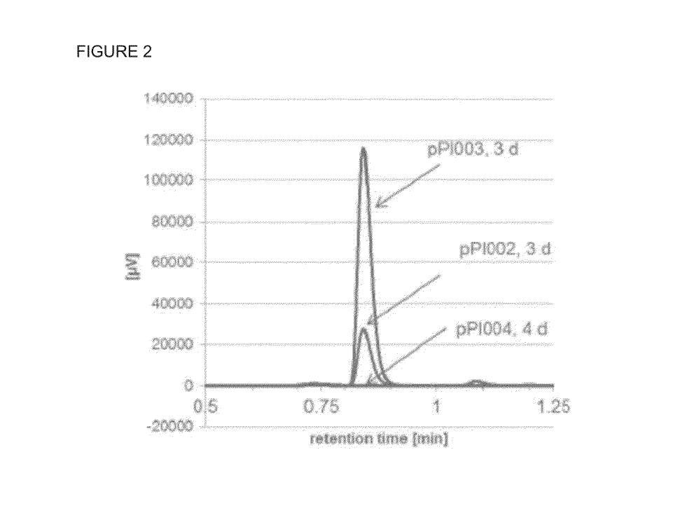

Butadiene formation was tested with the periplasmic and cytosolic linalool dehydratase from C. defragrans (pPI002 and pPI003) and LDHCg1 (from Colletotrichum gloeosporioides, pPI004). Both constructs of the linalool dehydratase from C. defragrans showed significant 1,3-butadiene formation after 3 days incubation (FIG. 2). The putative linalool dehydratase from Colletotrichum gloeosporioides (pPI004 LDHCg1) however, did not show any butadiene formation after up to 4 days incubation.

TABLE 2
Comparison of the different (putative) linalool dehydratases
tested in this study. Only the linalool dehydratase of C. defragrans
showed 1,3-butadiene formation from 3-buten-2-ol. Table 2 discloses
“His₆” as SEQ ID NO: 39.

| | | Activity | myrcene | 3-buten- | |
|---|---|---|---|---|---|
| | | in cell- | formation | 2-ol | |
| | | based | from | degra- | Buta- |
| | | assay | linalool | dation | diene |
| | Plasmid | (→ | (‘natural | during | for- |
| Organism | ID | growth) | substrate’) | growth | mation |
| C. defragrans with | pPI002 | Yes | Yes | Yes | Yes |
| periplasmatic tag | | | | | |
| C. defragrans with- | pPI003, | No | Yes | Yes | Yes |
| out periplasmatic | pPI010 | | | | |
| tag | (with C- | | | | |
| | His₆-tag) | | | | |
| Colletotrichum | pPI004 | Yes | No | Yes | No |
| gloeosporioides | | | | | |
| Neofusicoccum | pPI005 | No | No | n.d | n.d |
| parvum | | | | | |
| Thauera | pPI006 | No | No | n.d | n.d |
| linaloolentis | | | | | |
| Colletotrichum | pPI007 | No | No | n.d | n.d |
| gloeosporioides | | | | | |
| Mycobacterium sp. | pPI008 | No | No | n.d | n.d |
| JDM601 | | | | | |
| Oscillochloris | pPI009 | No | No | n.d | n.d |
| trichoides | | | | | |

Only the linalool dehydratase from C. defragrans was active towards the desired reaction (conversion of 3-buten-2-ol to 1,3-butadiene). The cytosolically expressed enzyme (pPI003), showed higher butadiene formation than the one with a periplasmic tag. The enzyme from Colletotrichum gloeosporioides was able to degrade 3-buten-2-ol, but no butadiene formation could be detected. Additionally, it was inactive towards linalool. All other alternative candidates showed no activity towards either 3-buten-2-ol or linalool.

Based on these results, the linalool dehydratase from C. defragrans without a periplasmic tag was chosen as the template for enzyme engineering. In order to allow for the quantification of the enzyme by blotting, a C-His6-tag (SEQ ID NO: 39) was added before the first round of engineering (construct pPI010, see Table 1).

### Example 3

### Construction of the Disclosed Polypeptides

All amino acid numberings described herein refer to the numbering of the originally published sequence of the wild-type linalool dehydratase from C. defragrans (Genbank Accession E1XUJ2.1, s. sequence 1 in the SEQUENCE LISTING) which, contrary to all variants described herein, contains a periplasmic tag. Thus, H83A on sequence E1XUJ2.1 corresponds to H58A on a sequence without a periplasmic tag.

The first round of engineering consisted of a carefully selected set of single mutants with the goal of identifying hot-spot positions amenable for engineering and testing the general applicability of such an engineering strategy.

Library Construction

First Round of Engineering: The first library, containing 93 single mutants, was constructed with a c-LEcta proprietary AGM method. The wild-type gene from C. defragrans without a periplasmic tag but including a C-His6 tag (SEQ ID NO: 39) was used as a template (Plasmid ID: pPI010, s. sequence 2 and 3 in the SEQUENCE LISTING). Origami2(DE3) was chosen as expression host, because previous studies had reported issues with soluble expression in BL21(DE3).

All variants were tested for myrcene formation as described in Example 4. In this experiment, pellets of expression cultures of cells harbouring the different linalool dehydratase variants were concentrated 10-fold for the assay.

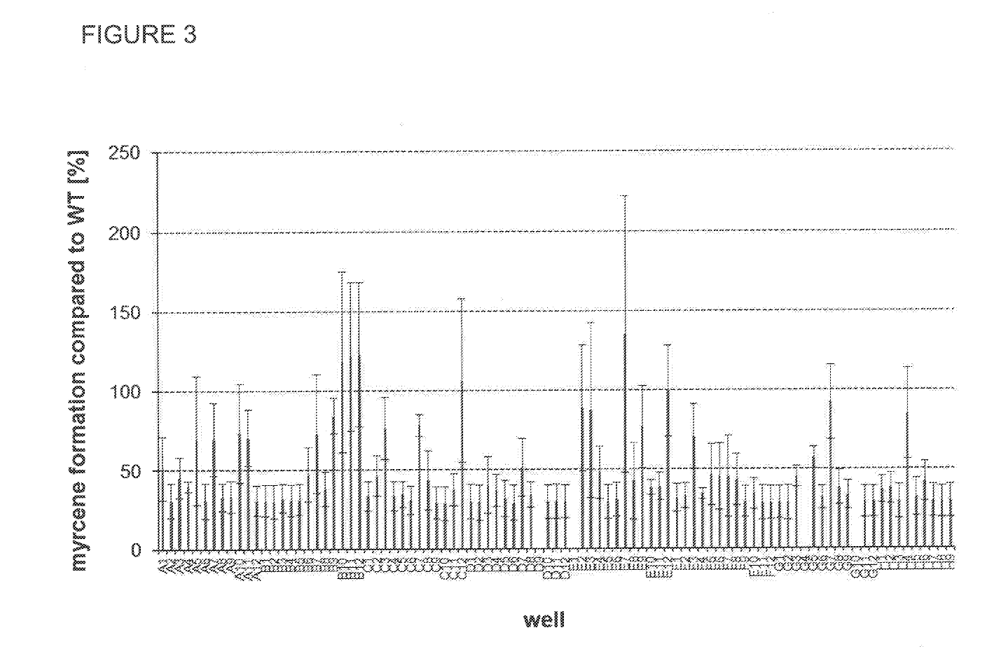

Several variants were identified, which showed an increase in myrcene formation compared to the wild-type (FIG. 3). Additionally, the vast majority of variants was active. However, as the error bars indicate, an unusually high variance between replicate experiments was observed, which shows that the screening assay suffers from quite a high error margin. Random testing of a number of expression cultures revealed higher differences in growth with Origami2(DE3) than are usually observed with BL21(DE3). This suggested that the high deviations in the assay might stem from uneven growth of Origami2(DE3) in deep-well plates.

Based on these results, a selection of nine variants was expressed in shaking flasks and tested for butadiene formation as described in Example 4. All tested variants showed significantly higher butadiene formation than the wild-type enzyme (Table 1). The best two variants, H83A and H252A, both exhibited a nearly 3-fold enhancement in butadiene production within two days which increased to 3.8-fold after five days (later experiments showed that the 3-fold improvement of the best variants resulted from improved soluble expression rather than an increase in specific activity.

TABLE 3
All variants showed higher butadiene
formation than the wild-type enzyme

| | | | | butadiene | |
|---|---|---|---|---|---|
| | | | | formation | |
| | | peak area butadiene, mean | | in % com- | |
| Plasmid | | value of two replicates ± σ | | pared to WT | |
| ID | mutant¹⁾ | 2 d | 5 d | 2 d | 5 d |
| pPI010 | — | 37756 ± 2317 | 64979 ± 7 | 100 | 100 |
| | H83Q | 57651 ± 1456 | 154048 ± 3438 | 153 | 237 |
| pPI011 | H83A | 109392 ± 2602 | 247288 ± 740 | 290 | 391 |
| | V103A | 68610 ± 3276 | 154622 ± 789 | 182 | 238 |
| pPI031 | V122I | 87625 ± 4058 | 173389 ± 4605 | 232 | 267 |
| pPI032 | D137R | 97440 ± 2280 | 220227 ± 1191 | 258 | 339 |
| pPI033 | S168D | 55312 ± 2538 | 118633 ± 1675 | 146 | 183 |
| pPI023 | R169D | 76914 ± 38 | 156585 ± 36636 | 204 | 241 |
| pPI024 | I186M | 92446 ± 2730 | 152675 ± 3928 | 245 | 235 |
| pPI012 | H252A | 111681 ± 1812 | 243895 ± 15 | 296 | 375 |

(Note:
At this point of the project, the effect on soluble expression of the host Origami2(DE3) had not yet been clearly elucidated; later experiments showed that the 3-fold improvement of the best variants resulted from improved soluble expression rather than an increase in specific activity. Butadiene formation of the 9 chosen primary hits compared to the wild-type enzyme (pPI010) in Origami2(DE3). For the assay, expression cultures were resuspended in M9 medium to 160 mg/mL,
¹⁾amino acid numbering refers to sequence E1XUJ2.1 (i.e. with a periplasmic tag)

The progression of butadiene production was tested with the C. defragrans linalool dehydratase variant H83A (pPI011). For the assay pellets of an expression culture of BL21(DE3)_pPI011 was suspended in M9-medium to a concentration of 160 mg/mL and butadiene formation was tested as described in Example 4.

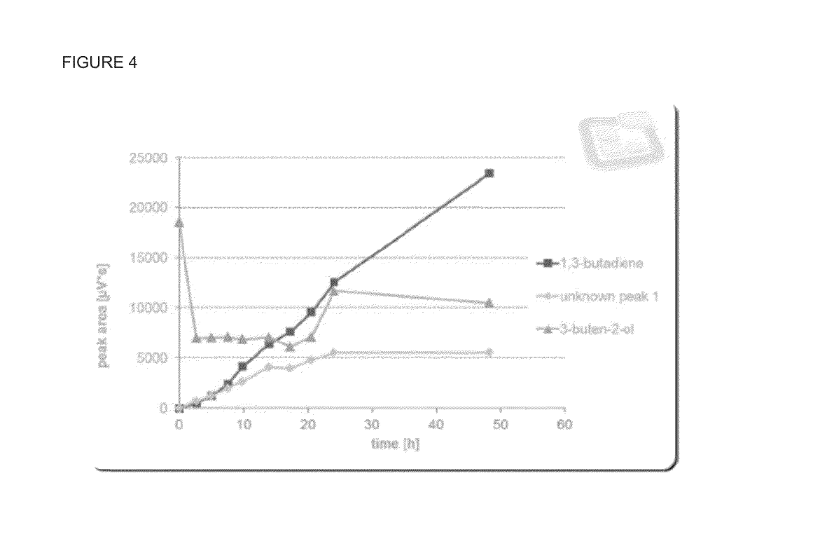

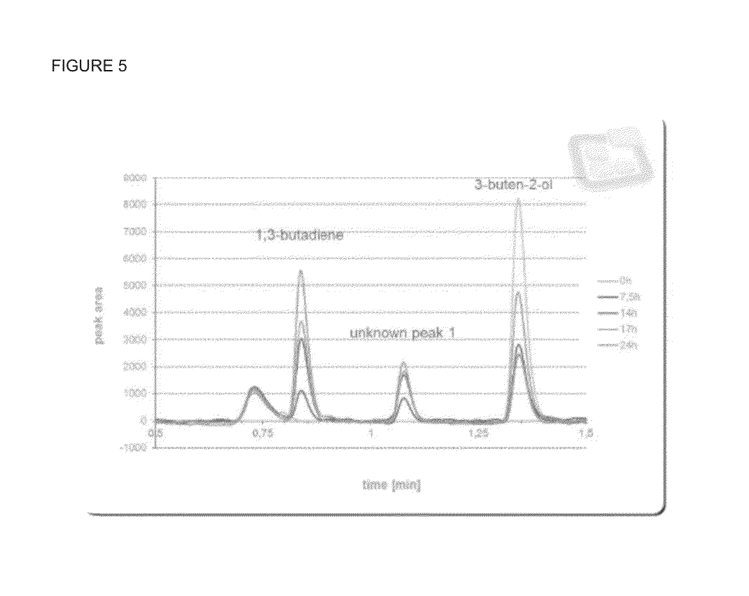

Butadiene production was fairly linear over the first 48 h (FIG. 4). Interestingly, the formation of a second product with a retention time of 1.08 min could be observed (unknown peak 1; FIG. 5).

Second Round of Engineering: For the second round of engineering, the two best variants from the first round of engineering as well as a double mutant combining these two variants were chosen as new templates. The three templates were combined with one additional mutation at the positions listed in Table 4. The additional positions were chosen either based on the results of the first round of engineering or because they were previously suspected as beneficial mutations. In order to increase the diversity of the new library, a partial saturation of the positions was chosen leading to 465 distinct variants (Table 4).

The library was constructed using the c-LEcta proprietary AGM method. As a host, BL21(DE3) was chosen because Origami2(DE3) showed uneven growth in the previous round of engineering that hindered accurate data evaluation in the screening.

TABLE 4
Selection of additional mutations in the second round
of engineering (1) amino acid numbering refers to sequence
E1XUJ2.1 (i.e. with a periplasmic tag).

| Wild-type | posi- | | |
|---|---|---|---|
| amino acid | tion¹⁾ | variant amino acid | reason for selection |
| V | 103 | ARNDCGHILFPSTYV | results from 1^{st} round |
| | | | of engineering |
| V | 122 | ARNDCGHILFPSTYV | results from 1^{st} round |
| | | | of engineering + |
| | | | Arzeda mutant |
| D | 137 | ARNDCGHILFPSTYVW | results from 1^{st} round |
| | | | of engineering |
| S | 168 | ARNDCGHILFPSTYVQ | results from 1^{st} round |
| | | | of engineering |
| R | 169 | ARNDCGHILFPSTYV | results from 1^{st} round |
| | | | of engineering |
| I | 186 | ARNDCGHILFPSTYVM | results from 1^{st} round |
| | | | of engineering |
| M | 273 | ARNDCGHILFPSTYV | Arzeda mutant |
| A | 323 | ARNDCGHILFPSTYV | Arzeda mutant |
| R | 359 | ARNDCGHILFPSTYV | Arzeda mutant |

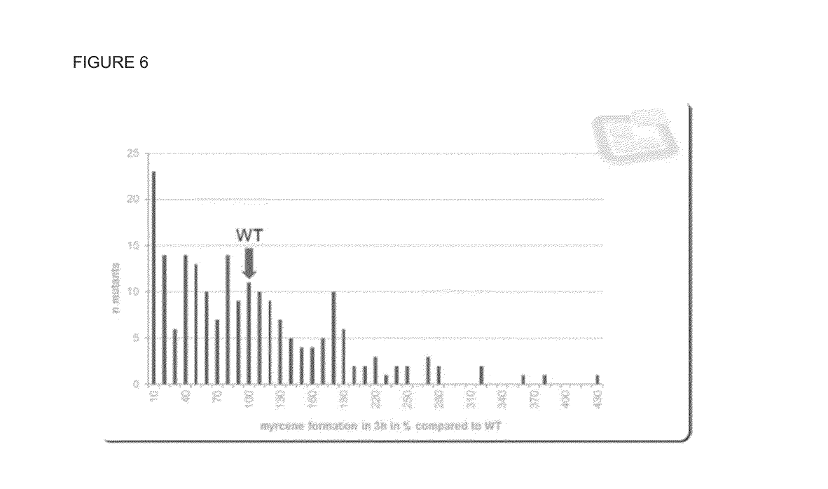

480 clones were screened for myrcene production analogues to the first library, as described in Example 4. A great number of clones were identified which showed higher myrcene formation than the wild-type (FIG. 6). Due to the method of library construction (i.e. the use of degenerate primers), several primary hits were found repeatedly. Sequencing revealed 9 distinct single and double mutants which were chosen for further characterization (Table 5).

TABLE 5
None of the selected primary hits from the second round of
engineering showed a significant increase in butadiene formation
compared to WT = pPI010 (1) amino acid numbering refers
to sequence E1XUJ2.1 (i.e. with a periplasmic tag).

| | | peak area | butadiene formation in |
|---|---|---|---|
| Plasmid ID | mutant¹⁾ | butadiene | % compared to WT |
| pPI010 | WT | 60400 | 100 |
| pPI011 | H83A | 68827 | 114 |
| pPI015 | H83A, R169S | 63466 | 105 |
| pPI016 | H83A, R169G | 61432 | 102 |
| pPI018 | H83A, I186C | 69073 | 114 |
| pPI020 | H83A, R359S | 51775 | 86 |
| pPI021 | H83A, R359L | 45648 | 76 |
| pPI022 | R169H | 59379 | 98 |
| pPI023 | R169D | 70613 | 117 |
| pPI024 | I186M | 66026 | 109 |
| pPI025 | R259S | 55382 | 92 |

Butadiene formation was tested with the selected nine variants as described in Example 4. For comparison, the wild-type enzyme and the single mutant H83A (pPI011) were included as well. None of the tested variants showed any improvement compared to the wild-type (Table 5). Additionally, H83A (pPI011), which had been identified as a beneficial mutation in the first round of engineering, did not show increased butadiene formation compared to the wild-type in this experiment (compare Table 5 and Table 3). In contrast to the screening in the first round of engineering, BL21(DE3) was used as the host. It was therefore decided to test the effect of Origami2(DE3) and BL21(DE3) on enzyme expression and activity.

Expression of variants from the first round of engineering in Origami2(DE3) and BL21(DE3): Previous experiments had shown that the expression of the wild-type enzyme was similar in BL21(DE3) and Origami2(DE3). However, the results from the second round of engineering suggested that expression of mutant enzymes might be different in the two hosts.

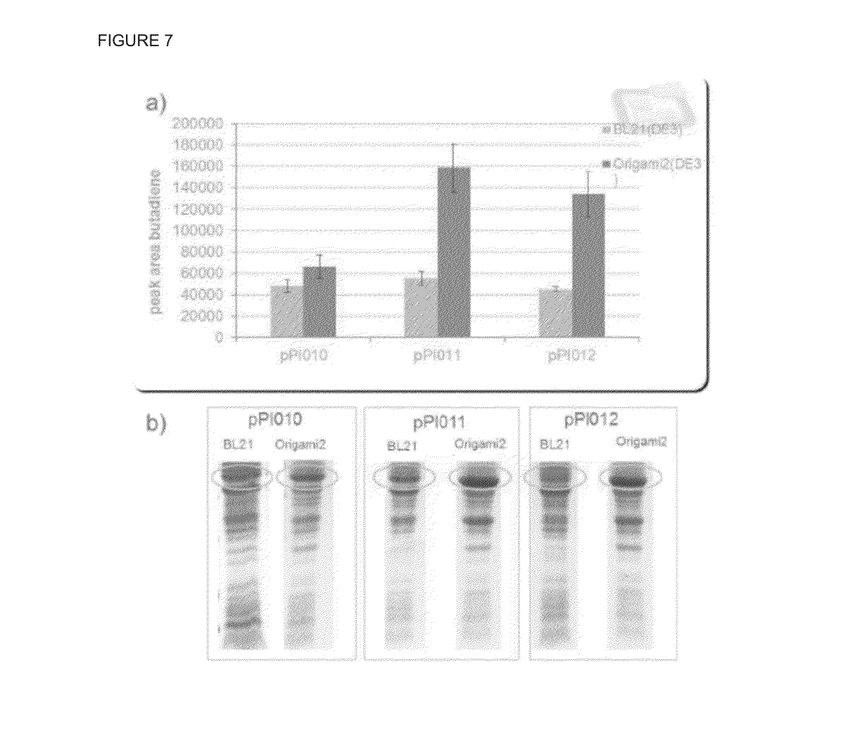

The wild-type enzyme and the variants H83A (pPI011) and H252A (pPI012) were tested for butadiene formation and soluble expression in Origami2(DE3) and BL21(DE3). Direct comparison of soluble expression and butadiene formation confirmed that the wild-type enzyme (pPI010) showed similar soluble expression in the two different hosts (FIG. 7). In contrast, the two single mutants H83A (pPI011) and H252A (pPI012) from the first round of engineering both exhibited a much higher soluble expression in Origami2(DE3) than in BL21(DE3) which results in significantly increased butadiene formation. These results suggest that the mutations did not lead (as desired) to an increased specific activity of the enzyme, but to an increased soluble expression.

Conclusions from the first two engineering rounds: In the first round of engineering, in which Origami2(DE3) was used as a host, several variants could be identified which showed improved butadiene formation compared to the wild-type enzyme. This improvement however, stems from improved soluble expression of these variants, which only appears in Origami2 (DE3) and not in BL21(DE3), and is hence not an enhancement of the specific activity.

The second round of engineering was done in BL21(DE3). The second round was based on hits from the first round of which at least the two best mutants only possess increased solubility in Origami. Additionally, all variants which exhibited improved activity towards the natural reaction (myrcene-formation) in the second round of engineering did not possess an increased activity towards the desired reaction (butadiene formation). Expression levels were more consistent in BL21 than in Origami.

Reassessment of Variants from First Round of Engineering

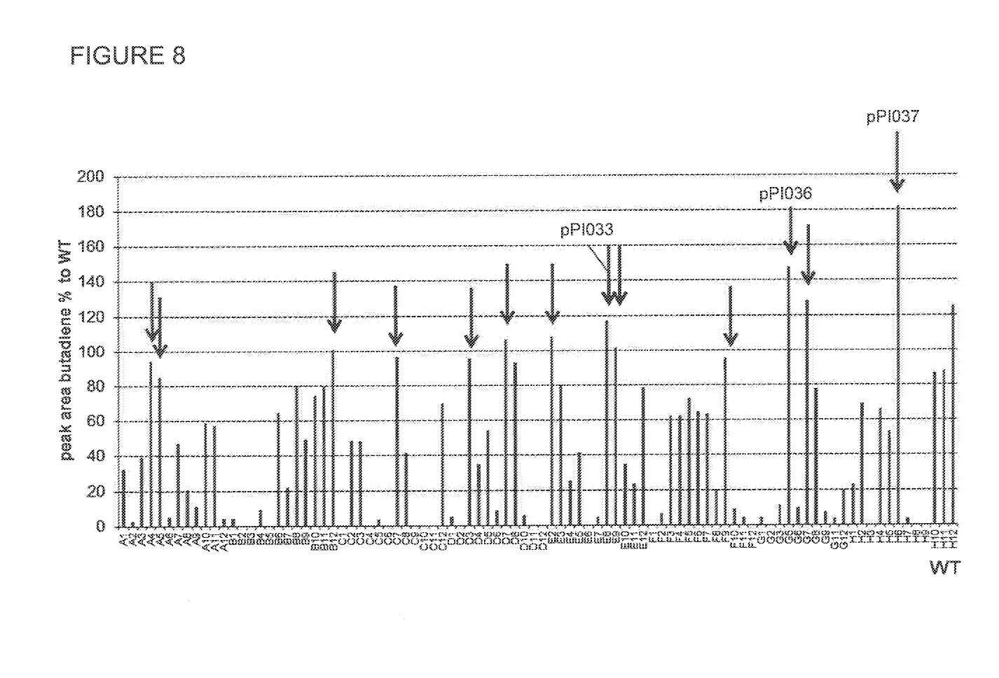

In order to reassess the variants of the first round of engineering, all variant plasmids were isolated from Origami2(DE3) and transformed into BL21(DE3). The amount of expression culture needed was produced by ten parallel expression cultures in deep-well plates which were subsequently pooled for the reaction. Butadiene formation was tested using the standard assay of Example 4 with an average cell density of OD600=63. The reaction was started and measured in eight consecutive batches so that the duration of the reaction was 48±0.5 h for all samples. Three primary hits could be identified, which showed between 20-80% increase in butadiene formation compared to the wild-type (FIG. 8, dark grey arrows). Ten further variants showed butadiene production similar to the wild-type enzyme (FIG. 8, light grey arrows).

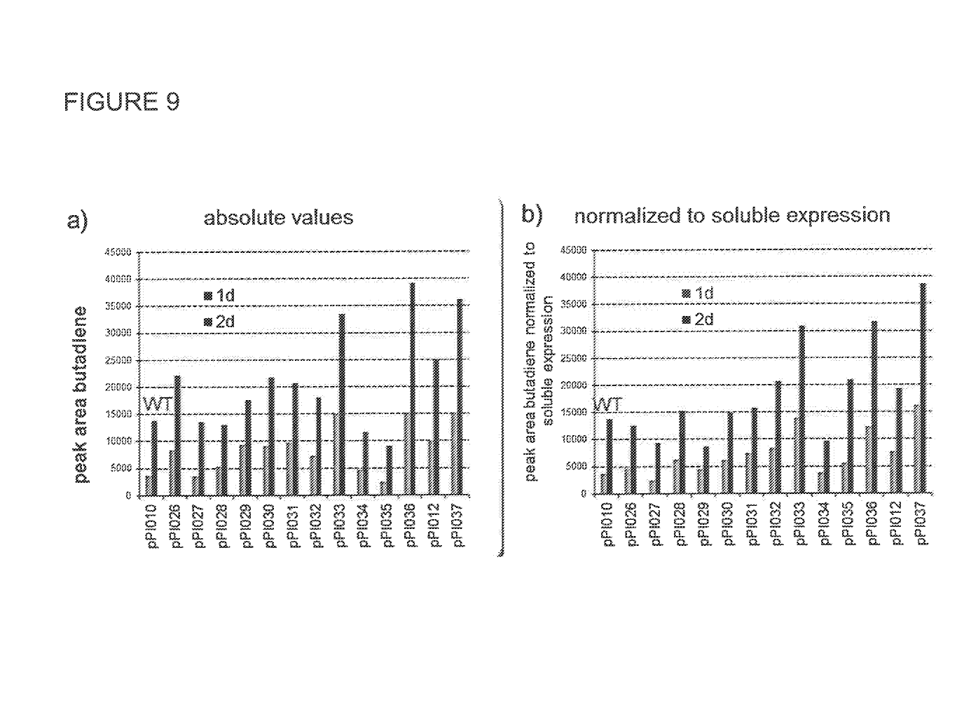

The three primary hits, together with 10 variants which showed significant butadiene production (FIG. 8, light and dark grey arrows) were chosen for further characterization. Butadiene formation was tested using the miniature assay (s. Table 4). The amount of soluble expression of each variant was assessed by SDS-page and quantified using the software Gelanalyzer2010. The measured peak areas were normalized to the amount of soluble expression. Three variants, could be identified which showed over 2-fold increase in butadiene formation compared to the wild-type enzyme (FIG. 9). Remarkably, only two of them corresponded to the primary hits found in the previous experiment (pPI036 and pPI037 in FIG. 8). The third hit (pPI033) had not shown significant improvements compared to the wild-type in the primary screening (compare FIG. 8 and FIG. 9). Additionally, normalizing butadiene formation to the amount of soluble expression of each variant revealed that some putative hits only exhibited increased soluble expression and no improvement in specific activity (e.g. pPI026).

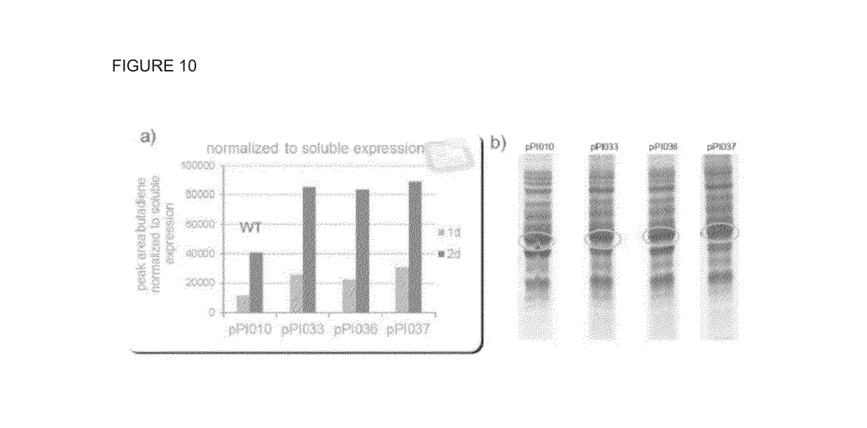

In order to confirm the results from the miniature assay, the three hits (pPI033, pPI026 and pPI037) were tested using the standard assay for butadiene formation and the measured peak areas were normalized to the amount of soluble expression as described above (FIG. 10, Table 6). Again, all three variants exhibited over 2-fold increase in butadiene formation compared to the wild-type enzyme. As can be seen in FIG. 10b, they showed very similar soluble expression compared to the wild-type enzyme; thus, the observed increase in butadiene formation results from an increased specific activity.

TABLE 6
Enhancement of butadiene formation of the three identified
variants compared to the wild-type. (1) amino acid numbering
refers to sequence E1XUJ2.1 (i.e. with a periplasmic tag)

| | | butadiene formation normalized to enzyme | |
|---|---|---|---|
| | | expression in % compared to WT | |
| Plasmid ID | Mutation¹⁾ | 1 d (=20 h) | 1 d (=20 h) |
| pPI010 (WT) | — | 100 | 100 |
| pPI033 | S168D | 219 | 209 |
| pPI036 | A230E | 191 | 204 |
| pPI037 | L366V | 264 | 218 |

These results show that selection and mutation of residues according to the annotated lead-sequence (built by using c-LEcta's MDM approach), yielded variants of the linalool dehydratase from C. defragrans with an approximately 2-fold increased specific activity (Table 6, FIG. 10).

### Example 4

### Screening for Variants with Desirable Activities

Detection of Myrcene Formation from Linalool by HPLC

96 deep well plates with expression cultures of mutant libraries were centrifuged (20 min, 4° C., 3,275×g), the supernatant discarded and the cells suspended in 50 mM TrisHCl pH 9 at 1/10 or ⅕ of the culture volume as specified. 100 μL/well of resting cells were transferred to a 96-deep well plate. After the addition of 80 μL/well 50 mM TrisHCl pH9 and 20 μL/well of a 100 mM linalool stock solution in EtOH (cfinal=10 mM), the plate was sealed with a solvent-resistant sealing tape (Steinbrenner #SL_AM0550) and incubated at 30° C., 300 rpm. After 3 h the plate was incubated on ice for 2 min, the reaction was quenched and the samples pre-pared for HPLC analysis as described in 3.5. HPLC analyses were always performed on the same day in order to avoid product evaporation.

Peak areas of myrcene formation were compared to peak areas of the WT-enzyme (the WT-enzyme was always present at least twice on the same plate).

Detection of 1,3-butadiene Formation from 3-buten-2-ol by Head-Space GC

For the detection of 1,3-butadiene formation from 3-buten-2-ol, expression cultures were centrifuged (3,275×g, 20 min, and 4° C.) and the pellet suspended in M9-medium either to equal OD600 (88.9) or equal biomass/mL (160 mg/mL) as specified. M9-medium: 200 mL of sterile 5×M9 salt solution (3.2 g Na2HPO4*7H2O, 7.5 g KH2PO4, 1.25 g NaCl, 2.5 g NH4Cl in 500 mL) were added to 10 mL of sterile 100×M9 additives (1.2 g MgSO4, 73 mg CaCl2*2H2O, 10 g glucose, 1.7 g thiamine HCl om 50 mL) and the volume was made up to 1 L with distilled water. Cell suspensions were transferred to a 1.5 mL HPLC vial. After addition of 100 μL of a 300 mM 3-buten-2-ol solution (cfinal=20 mM), vials were sealed immediately and incubated at 30° C., 120 rpm under a fume hood. Butadiene formation was analyzed by headspace GC as described below and the peak areas compared to those produced by the WT-enzyme.

GC Head-Space Analysis of 1,3-butadiene

Preparation of calibration standards: Butadiene calibration standards were prepared on ice on the day of the analysis as follows: from a freshly prepared 20 mM stock solution in hexane (prepared from a 15% solution in hexane, Sigma #695904) calibration standards were prepared directly in 50 mM Tris HCl-buffer pH 9 in GC-vials and sealed immediately. Without any incubation, an aliquot of the gaseous head-space above the sample was directly injected into the GC apparatus with a gas-tight syringe. (Due to the extremely low solubility of butadiene in water, it easily goes into the gaseous head-space of the vial without additional heating. Analysis of three replicates stored at 4° C., RT or 30° C. prior to analysis showed no differences in butadiene peak areas).

GC-FID operating conditions: Separation was achieved on a ZB1 column (Phenomenex, 1.0 μm thickness, 0.32 mm ID, 15 m length). The operating parameters were as the follows: split injection (split ratio=5), 8 μL injection, injection port temperature 200° C.; column temperature 50° C. for 10 min, FID-detector 200° C.

### Example 5

### Miniature Assay for Butadiene

By using a combination of lower cell densities and smaller reaction volumes (=the miniature assay), the screening throughput was substantially increased, because the amount of expression culture needed for the assay can be supplied by cultivation in 96-deep well format (Table 7).

TABLE 4
Comparison of the standard and miniature
assay for butadiene formation.

| | standard assay | | miniature assay | |
|---|---|---|---|---|
| Vᵥᵢₐₗ = | 1.925 | mL | 0.41 | mL |
| V_{Headspace} = | 0.425 | mL | 0.11 | mL |
| V_{Assay} = | 1.5 | mL | 0.3 | mL |
| cell density | OD₆₀₀ = 83 in the assay | | OD₆₀₀ = 56-75 in the assay | |
| resting cells = | | | | |
| incubation = | 30° C., 120 rpm, 2 d | | 30° C., 300 rpm, 2 d | |
| V_{expression culture} | ~10 | mL | ~1.4-2 | mL |
| for one assay = | | | | |

### Example 6

### Linalool Conversion to Myrcene

A number of polypeptides that differ from the polypeptide of SEQ ID NO. 11 in at least one position were tested for myrcene formation. 96 deep well plates with expression cultures of mutant polypeptides and control wild type LDH were centrifuged (20 min, 4° C., 3,275×g), the supernatant discarded and the cells suspended in 50 mM TrisHCl pH 9 at 1/10 or ⅕ of the culture volume as specified. 100 μL/well of resting cells were transferred to a 96-deep well plate. After the addition of 80 μL/well 50 mM TrisHCl pH9 and 20 μL/well of a 100 mM linalool stock solution in EtOH (cfinal=10 mM), the plate was sealed with a solvent-resistant sealing tape (Steinbrenner #SL_AM0550) and incubated at 30° C., 300 rpm. After 3 h the plate was incubated on ice for 2 min, the reaction was quenched and the samples pre-pared for HPLC analysis.

HPLC analyses were always performed on the same day in order to avoid product evaporation.

Chromatographic System:

Separations were carried out with gradient elution at 2 mL/min on a 4.6×150 mm Gemini 5 μm C18 110 Å column (Phenomenex) at 35° C. The mobile phase consisted of A=triethylamin acetate buffer pH 6.5 and B=acetonitrile. Gradient elution was as follows: 50% B for 3.75 min, increase to 90% B in 1 min, 1 min at 90% B, 90-50% B in 1 min and 3 min equilibration with 50% B. The injection volume was 5 μL. Linalool and geraniol were detected at 210 nm, myrcene at 230 nm

Sample Preparation

6.4 μL 1.25 M HCl were added to a 200 μL sample in order to lower the pH to approx. 3 and quench the enzymatic reaction. After vortexing, the pH was neutralised by addition of 4 μL 0.4 M NaOH. 200 μL of 50/50 (v/v) MeOH/50 mM TrisHCl pH 9 were added to the reaction mixture, followed by incubation on a rotary shaker at RT for 5 min. After centrifugation, the supernatant was transferred to HPLC-vials and submitted to analysis.

Validation.

For the evaluation of linearity and the lower limit of quantification, samples with various amounts of the respective analyte (0.05-5 mM geraniol, 0.05-5 mM myrcene and 0.05-10 mM linalool, n=2) were prepared in 50 mM Tris HCl-buffer pH 9. The lower limit of quantification was 0.05 mM for all analytes. The linear range was 0.05-2.5 mM for myrcene, at least 0.05-5 mM for geraniol and at least 0.05-10 mM for linalool

Peak areas of myrcene formation were compared to peak areas of the WT-LDH (the WT-enzyme was always present at least twice on the same plate).

| | | myrcene formation in | | butadiene |
|---|---|---|---|---|
| | | 3 h [in % compared to | | formation |
| Plasmid | | WT] using cells from | peak area | in % com- |
| ID | mutant1) | equal culture volume | butadiene | pared to WT |
| pPI010 | WT | 100 | 60400 | 100 |
| pPI011 | H83A | | 68827 | 114 |
| pPI015 | H83A, | 377 | 63466 | 105 |
| | R169S | | | |
| pPI016 | H83A, | 270 | 61432 | 102 |
| | R169G | | | |
| pPI018 | H83A, | 124 | 69073 | 114 |
| | I186C | | | |
| pPI020 | H83A, | 211 | 51775 | 86 |
| | R359S | | | |
| pPI021 | H83A, | 217 | 45648 | 76 |
| | R359L | | | |
| pPI022 | R169H | 319 | 59379 | 98 |
| pPI023 | R169D | 319 | 70613 | 117 |
| pPI024 | I186M | 427 | 66026 | 109 |
| pPI025 | R359S | 227 | 55382 | 92 |

1)Amino acid numbering refers to the sequence E1XUJ2.1 from Genbank (i.e. with a periplasmic tag)

### Example 7

### Conversion of 3-Methyl-3-Buten-2-Ol to Isoprene

For this assay, an alternative purification protocol was used. From a fairly fresh LB plate containing the desired clone transformant, one colony (or small scratch) was picked to inoculate 10 to 50 mL of LB supplemented with the relevant antibiotic and the pre-culture was incubated overnight at 37° C., 230 rpm.

The following morning, prepare the TB auto-induce medium (Merck/Code product: 71491-5) by mixing 60 g TB/L supplemented with 10 mL Glycerol/L of TB and microwaved during 3+2 minutes at full power. Let the TB cool down under the hood before using it and splitting it in sterile flasks. Then, Spin down the pre-culture incubated overnight and discard the supernatant. Resuspend the preculture in 1 to 5 mL of freshly prepared TB medium and use it to inoculate 100 to 500 mL of TB dispensed in the sterile flasks, supplemented with the appropriate antibiotic. Incubate the flasks of inoculated flasks at 28-30° C. for at least 20 h, 230 rpm.

The main culture was centrifugated at least at 3000 g/20 min/4° C. and the pellets used immediately. The pellets were resuspended in 10 to 20 mL of Buffer A (=50 mM Tris+150 mM NaCl+40 mM Imidazole+5% Glycerol−pH 8.5).

The resuspended cells were then sonicated in ice for 5 min at 35-40% Amplitude with 5″ ON and 15″ OFF sonication pulse. The sonicated cells were centrifugated at least at 15500 g, 20 min at 4° C. The supernatant containing the soluble fraction of proteins was recovered and used for His-trap protein purification.

The filtered soluble fraction of proteins obtained after extraction of proteins by sonication was used for His-tag protein purification. A 1 mL His-trap (GE Healthcare/Code product: 17-5319-01) column was equilibrated with 5-10 volumes column (VC) using Buffer A*. The soluble fraction of proteins was loaded onto the His-trap column manually using a syringe and 5-10 VC of Buffer A were used to wash the His-trap column. 5-10 VC of Buffer B** were used to elute the His-tagged protein directly to a 4 or 20 mL centrifugal filtration unit (VWR/Code product: 512-2850) with a relevant cut-off (5 kD). The centrifugal unit was spinned at 3500 g/5° C. to a volume lower than 400 uL concentrate. Around 3 mL of Buffer C*** was added to the concentrate and the centrifugal unit was again spinned at 3500 g/5° C. to a volume lower than 400 uL. This step was made to remove most of the imidazole used in Buffer B to elute the His-tagged protein. * Buffer A=50 mM Tris+150 mM NaCl+40 mM Imidazole+5% (v/v) Glycerol−pH 8.5** Buffer B=Buffer A+400 mM Imidazole−pH8.5*** Buffer C=Buffer A without Imidazole−pH8.5

The concentrate was recovered and according to the working concentration (≈2 mg/mL), Buffer C was used to top-up to the desired volume. The concentration was checked using a Nanodrop spectrophotometer.

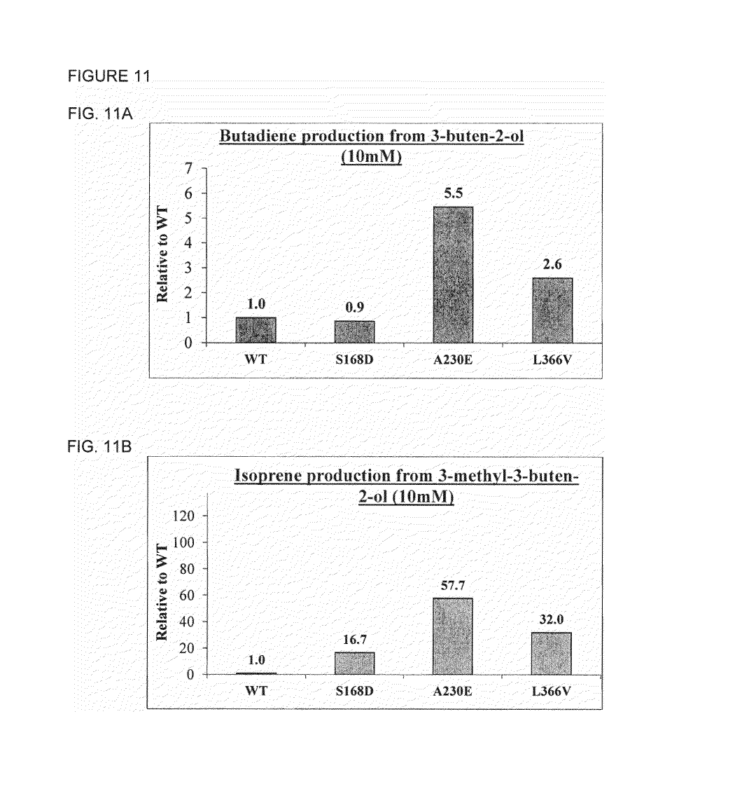

The purified proteins were used for butadiene assay. A 1 mL reaction made of 2 mg/mL of each purified enzyme with 10 mM of 3-buten-2-ol for the biosynthesis of 1,3-butadiene was prepared in a 1.7 mL crimped glass vial. The vials were incubated at least 48 h at 30° C., 170 rpm. The butadiene was analyzed by head-space GC-MS using an authentic standard to set up a standard curve for quantification. The results are shown in FIG. 11A. Mutants A230E and L366V all showed improved activity in dehydration of 3-buten-2-ol to butadiene, relative to WT cdLD.

Purified mutant polypeptides and WT control, were also tested for their ability to produce isoprene from 3-methyl-3-buten-2-ol. A 1 mL reaction made of 2 mg/mL of each purified enzyme with 10 mM of 3-methyl-3-buten-2-ol for the biosynthesis of isoprene was prepared in a 1.7 mL crimped glass vial. The vials were incubated at least 48 h at 30° C., 170 rpm. The isoprene was analyzed by head-space GC-MS using an authentic standard to set up a standard curve for quantification. The results are shown in FIG. 11B. Mutants S168D, A230E, and L366V showed increase isoprene-production activity, relative to WT cdLD.

### Sequences of Some of the Variants Disclosed

SEQ ID NO: 1:
>LDHCd with periplasmic tag and with C-His6-tag
(SEQ ID NO: 39), Linalool Dehydratase from
Castellaniella defragrans, codon-optimized.
ATGGGCTTTACCCTGAAAACCACCGCTATCGTGTCTGCGGCAGCCCT
TCTTGCTGGATTTGGACCTCCACCGCGTGCAGCCGAACTGCCACCTGGTC
GCTTGGCCACGACCGAGGACTATTTCGCACAACAGGCCAAACAAGCGGTT
ACTCCGGATGTGATGGCTCAACTGGCGTACATGAACTATATTGACTTTAT
CAGCCCCTTCTATTCTCGCGGTTGTAGCTTTGAGGCTTGGGAACTGAAGC
ATACCCCACAGCGCGTGATTAAGTACAGCATCGCGTTTTACGCTTATGGC
CTGGCAAGTGTGGCGCTGATTGATCCGAAACTGCGTGCGTTAGCCGGTCA
TGATCTCGACATTGCGGTGTCGAAAATGAAGTGCAAACGGGTATGGGGCG
ATTGGGAGGAAGATGGGTTCGGTACCGATCCGATCGAGAAAGAGAACATC
ATGTACAAAGGCCATTTAAACCTGATGTATGGGTTGTACCAGCTCGTAAC
AGGCAGTCGTCGCTATGAAGCCGAACACGCACATCTCACCCGCATCATTC
ACGATGAGATTGCGGCGAATCCTTTTGCGGGCATTGTGTGTGAACCGGAT
AATTACTTCGTTCAGTGCAATTCGGTGGCGTATTTATCCTTGTGGGTCTA
TGACCGGCTGCATGGTACTGATTACCGTGCTGCAACACGCGCATGGCTGG
ACTTCATCCAGAAAGACCTGATTGACCCGGAACGTGGTGCGTTCTACCTG
TCATATCACCCCGAATCTGGCGCAGTTAAGCCGTGGATTAGCGCGTATAC
GACAGCCTGGACGTTAGCGATGGTACACGGAATGGACCCGGCGTTTTCCG
AACGCTATTATCCGCGCTTTAAACAGACCTTCGTCGAAGTCTACGACGAA
GGCCGTAAAGCCCGTGTTCGCGAAACTGCCGGGACGGATGATGCCGATGG
TGGCGTTGGTCTGGCATCCGCGTTTACGCTGCTTCTGGCACGCGAGATGG
GCGATCAGCAACTGTTCGATCAGTTACTTAACCACTTGGAACCGCCCGCC
AAACCGAGCATTGTCTCAGCTAGTCTGCGCTATGAACATCCGGGGTCGTT
GCTCTTCGATGAACTGCTGTTTCTGGCAAAAGTGCATGCGGGCTTTGGTG
CCCTGTTACGTATGCCACCTCCGGCTGCCAAACTGGCGGGCAAACATCAT
CACCATCACCATTAA
SEQ ID NO: 2:
>LDHCd with periplasmic tag, Linalool Dehydratase
from Castellaniella defragrans, codon-optim ised
ATGGGCTTTACCCTGAAAACCACCGCTATCGTGTCTGCGGCAGCCCT
TCTTGCTGGATTTGGACCTCCACCGCGTGCAGCCGAACTGCCACCTGGTC
GCTTGGCCACGACCGAGGACTATTTCGCACAACAGGCCAAACAAGCGGTT
ACTCCGGATGTGATGGCTCAACTGGCGTACATGAACTATATTGACTTTAT
CAGCCCCTTCTATTCTCGCGGTTGTAGCTTTGAGGCTTGGGAACTGAAGC
ATACCCCACAGCGCGTGATTAAGTACAGCATCGCGTTTTACGCTTATGGC
CTGGCAAGTGTGGCGCTGATTGATCCGAAACTGCGTGCGTTAGCCGGTCA
TGATCTCGACATTGCGGTGTCGAAAATGAAGTGCAAACGGGTATGGGGCG
ATTGGGAGGAAGATGGGTTCGGTACCGATCCGATCGAGAAAGAGAACATC
ATGTACAAAGGCCATTTAAACCTGATGTATGGGTTGTACCAGCTCGTAAC
AGGCAGTCGTCGCTATGAAGCCGAACACGCACATCTCACCCGCATCATTC
ACGATGAGATTGCGGCGAATCCTTTTGCGGGCATTGTGTGTGAACCGGAT
AATTACTTCGTTCAGTGCAATTCGGTGGCGTATTTATCCTTGTGGGTCTA
TGACCGGCTGCATGGTACTGATTACCGTGCTGCAACACGCGCATGGCTGG
ACTTCATCCAGAAAGACCTGATTGACCCGGAACGTGGTGCGTTCTACCTG
TCATATCACCCCGAATCTGGCGCAGTTAAGCCGTGGATTAGCGCGTATAC
GACAGCCTGGACGTTAGCGATGGTACACGGAATGGACCCGGCGTTTTCCG
AACGCTATTATCCGCGCTTTAAACAGACCTTCGTCGAAGTCTACGACGAA
GGCCGTAAAGCCCGTGTTCGCGAAACTGCCGGGACGGATGATGCCGATGG
TGGCGTTGGTCTGGCATCCGCGTTTACGCTGCTTCTGGCACGCGAGATGG
GCGATCAGCAACTGTTCGATCAGTTACTTAACCACTTGGAACCGCCCGCC
AAACCGAGCATTGTCTCAGCTAGTCTGCGCTATGAACATCCGGGGTCGTT
GCTCTTCGATGAACTGCTGTTTCTGGCAAAAGTGCATGCGGGCTTTGGTG
CCCTGTTACGTATGCCACCTCCGGCTGCCAAACTGGCGGGCAAATAA
SEQ ID NO: 3:
>LDHCd without periplasmic tag, Linalool Dehydratase
from Castellaniella defragrans, codon-optim ised
ATGGCCGAACTGCCACCTGGTCGCTTGGCCACGACCGAGGACTATT
TCGCACAACAGGCCAAACAAGCGGTTACTCCGGATGTGATGGCTCAACTG
GCGTACATGAACTATATTGACTTTATCAGCCCCTTCTATTCTCGCGGTTG
TAGCTTTGAGGCTTGGGAACTGAAGCATACCCCACAGCGCGTGATTAAGT
ACAGCATCGCGTTTTACGCTTATGGCCTGGCAAGTGTGGCGCTGATTGAT
CCGAAACTGCGTGCGTTAGCCGGTCATGATCTCGACATTGCGGTGTCGAA
AATGAAGTGCAAACGGGTATGGGGCGATTGGGAGGAAGATGGGTTCGGTA
CCGATCCGATCGAGAAAGAGAACATCATGTACAAAGGCCATTTAAACCTG
ATGTATGGGTTGTACCAGCTCGTAACAGGCAGTCGTCGCTATGAAGCCGA
ACACGCACATCTCACCCGCATCATTCACGATGAGATTGCGGCGAATCCTT
TTGCGGGCATTGTGTGTGAACCGGATAATTACTTCGTTCAGTGCAATTCG
GTGGCGTATTTATCCTTGTGGGTCTATGACCGGCTGCATGGTACTGATTA
CCGTGCTGCAACACGCGCATGGCTGGACTTCATCCAGAAAGACCTGATTG
ACCCGGAACGTGGTGCGTTCTACCTGTCATATCACCCCGAATCTGGCGCA
GTTAAGCCGTGGATTAGCGCGTATACGACAGCCTGGACGTTAGCGATGGT
ACACGGAATGGACCCGGCGTTTTCCGAACGCTATTATCCGCGCTTTAAAC
AGACCTTCGTCGAAGTCTACGACGAAGGCCGTAAAGCCCGTGTTCGCGAA
ACTGCCGGGACGGATGATGCCGATGGTGGCGTTGGTCTGGCATCCGCGTT
TACGCTGCTTCTGGCACGCGAGATGGGCGATCAGCAACTGTTCGATCAGT
TACTTAACCACTTGGAACCGCCCGCCAAACCGAGCATTGTCTCAGCTAGT
CTGCGCTATGAACATCCGGGGTCGTTGCTCTTCGATGAACTGCTGTTTCT
GGCAAAAGTGCATGCGGGCTTTGGTGCCCTGTTACGTATGCCACCTCCGG
CTGCCAAACTGGCGGGCAAATAA
SEQ ID NO: 4:
>LDHCg1 codon-optimised nucleotide sequence from
putative linalool dehydratase ELA33010
(Colletotrichum gloeosporioides)
ATGGCAACCGCAACCATTACCAGCACCCAGACCAATAATGGCACCCT
GGAACTGCGTGGTGAAGCACCGAGCAAACTGCCGAAAACCCTGCCTGCAG
ATTTTATTGAACGTTTTCCGAAACTGAGCCGTGAACAGGCAGGTCATCTG
CGTCATTTTCATAATCTGGCAACCCAGAAAGATGGCGAATGGAAACACAT
GGGTAGCCAAGAACCGGGTCAAGAATGGCTGGATGCATATCGTTATCAGC
TGGCAACCATGGCATATGCAGCCGGTGCAGCACATTATCATCATCTGCCT
GCACTGCGTAGCACCTTTAAAAGCCTGCTGGAAAGCCTGATTCATAAAAT
GCTGCTGCGTGATGTTTGGGGTTATTGGTTTCTGACCAGCCATAGCGGTA
TTATGGTTGATCCGGATATTAAAGAACTGCGTAAACCGTGGGCAGATCCG
GTTGTTCGTGAAAACATTATGTATAGTGGCCATCTGCTGCTGATGGTTAG
CCTGCATGAAATGCTGTTTCATGAAGGTCGTTTTGATGATGAAGGTAGCA
TTGCCTTTAATTGGAACCCGATTTTTTGGGGTATGGGTCCGGAACGTTTT
TGTTATACCCGTAAAACACTGCAAGAAGCAATTCTGCGCGAAATGGAACG
TGAAAATTGGCTGGGTGTTTGTTGTGAACCGAATAGCATTTTTGTTGTGT
GCAATCAGTTTCCGCTGATTGCCCTGCGTTATAATGATGTGCGTGATAAA
ATTGATCTGAGTCCGGGTGTTCTGGAAAAATATCAGGCAGCATGGAAAAG
CAAAGGTATGATTAGTGATGATGGCCTGATTGTGGATTGGTATAGCCCGA
AACAGAATCGTACCAAACCGCCTAGCGATATTGCATTTACCAGCTGGGCA
CTGGCATTTATGAATAGCTGGAACCCGGATTTTGCACGTCGTACCAGCAA
AGATATTGCAATTGGTTATCTGGCCAAAAGCCATGAAGATCATGTTTTTG
TTCCGGATCCTGAAGTGAGCTTTAAAATCCGTGAACTGGTTGCAAGCGAA
CGTCTGGATCCGATGGATCCGGCAACCTATGCACGTGCCGCAAAAGCAGT
TGCAGAACAGAATCTGCCTGCAAGCGGTTTTCCGTTTACAAAACCGCATT
TTGCATATGCCGCAATGTGGGCCAGCGAACTGGGTGATCCTGAACATCTG
GATGGTCTGCTGGCCTATGCAGATGTTAAAATGAATCCGACCTGGGAAGA
TGGTGGTCTGTTTTATGGTGGTAGCGGTAAAAGCGAAGCAGCAAGCGGTG
TTGATGTTATTAGCGGTAATGCAGCAGTTGCGTATGCACGTTTTAATGTG
CCGGATGGTCAGCGTACCATGTATGAAAAACCGTGGGATGCAGAACATTT
TGCAACCGTTCCGTTTGTGAAAAATGTTGATCTGGCAAGTGGTGTGGATT
TTCTGCGTGGTAGCTGGGATGAAGAACTGCAGGCACTGGCCGTTACCCTG
CGTAGTTGGGATGGCACCAATAAAAGCGTTCAGCCGCAGTTTACCGGTCT
GCAAGAGGGTAATTATGGTATTTATCAGAATGGTGTGCTGCACCAGACCG
AAGAGGTTAAAAGCCGTGATGATGTGATTGCATTTGGTCTGCAGGTTAGC
GGTGATGAAGTGGATCTGGTTCTGGTTCGTAGCCATTAA
SEQ ID NO: 5:
>LDHNp codon-optimised nucleotide sequence from
putative linalool dehydratase E0D44468
(Neofusicoccum parvum)
ATGGCAAGCCAGACCGCAACCACCACCGCAGCACCGGGTAGCATTC
CGCTGAGCACACCGGGTCCGCTGCCGATTGCACTGCCGAGCCATATTCTG
AGCAAATTTCCGGCACTGACACCGGCACAGGCAGGTCATCTGCGTCATTTT
CATAATCTGGCAACCCAGCTGGATGGTGAATGGCGTCATATGGGTGCACAG
GATCCGGGTCAAGAATGGCTGGATGCATATCGTTATCAGCTGGCAACCATG
GCATATGCAGCCGGTGCAGCACATTATCATCGTCTGCCTGCACTGCGTAGC
GTTTTTCGTGTTCTGCTGGAACAGCTGATTCATAAAATGCTGCGTCGTGAA
GTTTGGGGTTATTGGTATCTGACCAGCCAGAGCGGTCGTTTTGTTGATCCG
GATATTGAAGAACTGCGTAAACCGTGGTCAGATCCGATTAAACGTGAAAAC
ATTATGTACAGTGGCCATCTGCTGCTGATGGTTAGCCTGCATGCAATGCTG
TTTGATGATGATAAATATGATCAGCCGGATGCCCTGGTTTTTGATTGGAAT
CCGATTTTTTGGGGTATGGGTCCGGAAAAATTCTGTTATAGCCGTAGCAGC
CTGCAGAAAGCAATTCTGGATGAAATGGAACGTACCAATTGGATGGGTGTT
TGTTGTGAACCGAATAGCGTGTTTGTGGTTTGTAATCAGTTTCCGCTGATT
GCCATTCGCTATAACGATGTTCGTAATGGCACCAATGTTATTGATGGTGTG
CTGGATAAATATCGTGCAGCATGGGATAGCCGTAATGGTTTTACCCAGGGT
GGTGATCAGATGGTTGCATGGTGGCGTCCGAAACAGCAGGATTTTGTTCCG
GGTAGCAGCATTGGTTTTAGCAGCTGGGCAAGCGCATTTATGAATGCATGG
AATCCGAGCTATTGTCATGCAATGTATCCGAGCTTTGCACTGGGTAATCTG
ACCCGTCATCCGAGCGGTCGTGTTAATCTGAATCCGCCTGCAGTTGCAGCA
GAAATTCGTGCACTGGTTCATGATGATCCGGCAACCGATCCGCATGCACCG
GCAACCCTGGATCGTGCACGTGGTCGTGCAGCCGAAAAAGCAGCAGCAGCC
GCAGCACGTCAGCAGCAACAGCCTCCGGGTCCGCCTAAACCGCCTGCAAGT
CCGGAATTTGGTTATGTTGTTAAATGGGTTATTAGTCCGGTGGTGAAAAAT
CTGCCTGCAGGTCTGTATGGTATTTATGAAGGTGGTAAACTGGTTCAGACC
CGTAGCACCGGTGGTGGTGATGGTGGTATTGATCTGGAACTGCAGGTTGGC
GGTGATGAACTGGATGTTGTTCTGCTGAAACAGAAATAA
SEQ ID NO: 6:
>LDHTI codon-optimised nucleotide sequence from
putative linalool dehydratase WP_004338616
(Thauera linaloolentis)
ATGGAAAGCACCCGTATGCTGCGTCAGCCGATTCAGCTGCTGCAGG
GTCATAAAGGTCCGGTTACCGCAAGCCGTCATCGTCGTAATGCAGTTGTT
TATGCACTGCTGTGTCTGCTGGCACTGCTGCCGGTTGCCACCGGTCAGAG
CGCAGCATGGCAGGCAGCAGGTCTGGGTCTGTTTATGCCTGGTGCAGGTT
TTCTGGCACTGGGTGGTGCATGGGCTCTGCTGTTTCCGCTGACCGTTTTT
GTTTTTTGGCTGGCAGTTATTGCATGGTTTTGGAGCGGTATGGTTGTTGC
ACCGCTGACCCTGTGGCTGGGCACCGCTGCACTGGCAGGATGGCTGGCTG
GTGAAGCAATTTGGCCTCCGGCAGTTTATCTGGCTCCGGCAGCCGCAGCA
GCAACCTTTCTGTTTTTTCAGTATCGTGGTGCAAAACGTCGTGCAAAAGA
TCGTGAACATTTCAAATTTCGCCAGAGCTTTTTTGCAGAAAGCCTGGCCG
AAGTTCATCAGCGTGCAGCAACCGAACCGGAACCGGGTGAACGTGAACTG
ACACCGGATCAGCTGCAGGGCGTTCGTTATCTGCTGGAACTGGCGCTGCA
GCCGGTGGGTCAGTATAAAGGTTATACCATTATCGATCAGTTTCAGCCTG
CAGCACTGCGTTATCAGCTGAACCATATTGGTTTTGCACTGGGTATGGTA
CAGGGTCACTATACCCCGAATTTTCAGGGTTATCTGGGTCAGGCACAGCG
TAATGTTATTGATACCTATCGTGAACGTAAAGTGTGGGGTTATTGGGTTT
ATGAAAGCATGTGGGGTCACTTTAACTTCAGCGATTTTGATCCGGCACGC
AAAGATAATATCATGCTGACCGGTTGGTATGGTATGCATGTTGGCCAGTA
TATGCTGAATGCCGGTGATACCCGTTATAGCCAGCCTGGTAGCCTGAGCT
TTCGTCTGAATGATAAGACCTGTTATCATCATGATATCCATAGCATTAAT
CAGAGCGTGCGTGAAAACTTTCAGAGCAGCGATTTTTGTCTGTATCCGTG
TGAACCGAATTGGGTTTATCCGGTTTGTAATATGTATGGTATGAGCAGCC
TGGCAGTTTATGATACCCTGTTTGAACGTCGTGATACCGCACAGGTTCTG
CCGAAATGGCTGCATATGCTGGATACCGAATTTACCGATCAGAAAGGTAG
CCTGGTTGGTCTGCGTAGCTATTGGACCGGTCTGGAAATGCCGTTTTATA
CCGGTGAAGCAGGTTTTGCATTTTTCGCCAACATTTTTAGCACCGATCTG
GCACGTAAACTGTGGGCAGTTGGTCGTAAAGAACTGAGCATGTGTCTGAC
CCAGGATGCAGAAGGTCAGACCCGTCTGACACTGCCGAAAGAAGCACTGG
CCTTTTTTGATACCATTGATGCAGGTAATTATCGCCCTGGTAAACTGTTT
GCATATGTTGCAGTTCAGATGTGTGCACGTGAATTTGGTGATGATGAACT
GGCAGAAGCAGCACGTCGTAGCATGGATCAGGATTGTGGTCCGGTTGTTG
AAAATGGTGTTGCACGTTATACCAAAGGTAGCACCCTGGCCAATATTTGG
GGTGTTGAAGGTCGTCTGATGCGTACCGGTGATTTTCGTAATAGCTTTGT
TAAAGGTCCGCCTAGCAGCGTGTTTGATGGTCCGCTGCTGGGTGATGCCC
GTTATCCGGAAATTCTGGTTGCAAAAGCATTTAGTCGCGGTGATGATCTG
GAACTGGTGCTGTATCCGGGTGCCGGTGATGGTCCTCAGACCCTGGGTTT
TGAACGCCTGAAACCGGGTGTTCGTTATGTTGTGGAAGGTGCAGCAAGCG
GTGAATTTACAGCAGATGCAGATGGTCGTGCAAGCCTGGCCGTTACCCTG
AGCGGTCGTACCGCACTGCATATTAAACCGGGTCATTAA
SEQ ID NO: 7:
>LDHCg2 codon-optimised nucleotide sequence from
putative linalool dehydratase ELA28661
(Colletotrichum gloeosporioides)
ATGGCACCGAGCACCATGACCACCACCACCGAAACCACCAAAACCA
ATGGTGTTAATCATCTGGATGCAGCAGAACTGCCGAGCAAAATTGCACCG
AGTAGCAAATGGGTTGATACCAGCGATAGCATTAAAGCAGATCCGAGCAC
CTCAGTTAAAGCAAGTGATGGTCCGGTTGGTGATTTTGGTTGGGGTCCGT
GGAAAATTCAGGTTCCGGTTGAATATACCTTTCTGAGCCTGGCAGGTCTG
TGGTCATTTCATAATCTGGAAAGCACCAGCTATCGTGCAGCAGCACTGGG
TTTTCTGTTTCCGGGTGCAGGTTTTACCGCAGTTGCAAGCCCGACCGCAG
TGGCAGCATTTCTGCTGACCCTGGTTCTGATTCCGGTTAGCATTTTTGTT
TGGTTTGCAATGGGTGGTATTGCATTTCCGATTGCACTGTGGATTGGTAG
CAGCTTTATGGCAGGTCGTCTGGCACAGGATACCCTGTTTGAACAGAGCG
CAGCACTGTGGGCACTGGGCTGTTTTAGCGGTATTACCTGGCTGATGAAT
AATGCAAGCAGCCTGAATGCAGCAGGTTATAGCAAAGCACAAGAACGCAA
CAAATATCTGGTTCAGGCAGTTGAAGAACAAATGGCAGATGCAGCACCGG
CACCGCAGAGCGGTGATCGTGAACTGAGTCTGGAAACCCTGCGTCATGTT
CAGCATATGATTGAACGTGGTCTGAGTCCGCGTGATGATTTTAGCTTTCA
TGATGTGATTGATCAGTTTCAGACCGGTGCCATTCGTTATCAGCTGTATG
GCACCATTGATGCACTGAGCCTGTATCAGTGTCATTATGTTCCGGGTTTT
CATGGCTATCTGAGCAAAGCATGTCAGAACGCAATTGAAAAAAGCCTGCA
GAAACGCATTATGAGCTATTGGAAATGGGAAAGCATCTTTGGTCGTTTTA
CCCTGAGCGATTGGGATCCGATCAAAAAAGATAACATTATGGTGACCGGT
TATCTGAGCGCAGCCATTGGTCTGTACGGTCAGGCAAGCGGTGATCGTCA
GTATAACAAAAAAGATGCCCTGGAATTCGTGATCGATGATGGCAAACACT
ATAAAACCAATTATGAAGGTCTGGCCGATGCCCTGTTTAATAACATGACC
GAAAATCCGTATTGTCTGTATCCGTGTGAACCGAATTGGACCTATAGCCT
GTGTAATCTGACCGGTATGGCAGGTCTGGTTATTAGCGATCGTCTGCTGG
GTCGTGATCTGGGTGTTAAACTGCGTAATCGTTTTGAACGTAGCCTGGAA
GAGGAATTTACCGAATGTGATGGTCGTATTCTGCCGATTCGTAGCGAATT
TACAGGTCTGACCCTGCCTGGTCTGTGTGGCACCCTGACCGATTGTATTA
ATGCAATGCTGCTGACCGCATATCTGCCGCATCTGGCACATCGTAATTGG
GCAATGATTCGTAAAGAGTTCCTGAAATACGATAAAAACGGTCAGCTGGA
AGTTCGTAGCCTGAAAGGTGCAGATAAAATGGATCCGCGTAATTATCGTG
CAAGCGAAGGTCCGCTGCGTGCATTTATTGCAGCAACCGCAGCAGAATTT
GGCAATGAAAAAATTCGCAAAGAAGCACTGCATCAGCTGGATAATACCTA
TTTTCCGGTTGAAGCAACCAAAAGCGGTAGCCTGCGTAATAAACGTAGCG
GTCCTCTGCTGGAAGAGGCACCGTTTCCGGATGTTCTGGTTGCAAAAGCA
TATAGCAATGATGGTAAACAGCTGGATCTGGTGCTGTATAATGGTGCAGA
ACCGGGTACATTTGAACTGGGCTTTGAACGTCTGGTTCCGGGTAAAGAAT
ATTCACTGAGCACCGGTGGTAGCGTTAAAGCAAATAACAAAGGTAAAGCC
ACCGTTAAAGTTAGCGTGAAAGGTCGTACCCAGATTATTCTGAAACCGGT
TGTTTAA
SEQ ID NO: 8:
>LDHM codon-optimised nucleotide sequence from
putative linalool dehydratase YP_004525079
(Mycobacterium sp. JDM601)
ATGAGCGCACCGACCATTGAAAGCGAACAGAGCACCGATATTGGTTA
TGTTTTTGAAGTTCCGGATCGTCCGAATGGTCCGGCAGTTCGTCGTCTGC
TGCGTCGTAGCGGTGCACTGCTGGGTGCAGTTGGCACCGTTGCAACCCTG
ACCGCATGGCGTAGCAAACGTCCGCGTGTTCGTGCAATTGCCCTGGGTCT
GCTGGCACCGGGTGGTGGTCAGCTGTATACCCGTAGTCCGCTGCGTTTTG
TTGCAACCGTTGCAGGTTTTTTTGCAAGCCTGGTTGCATGGTTTGGTAGC
GGTAACATTATTGCACCGGTTCTGGTTTGGCTGACCGCAGCAACCAATGC
AGGTAGCCATGCAGCCGGTGGTCGTCGTACCTGGAATGGTGCACGTCGTG
TTATTCCGACCGCAGTTGCCGGTGCAGCAGCAGCCGGTATTGTTGCCCGT
CGTCGTGCATTTCATGCAGCACAGGCACGTGGTCGTACCCGTGCAGAATA
TCTGACCACCGCACCGCGTCTGGATCCGCAGCCGAAAGATGCAGCAAGCG
AAGAACTGAGCCCGACCGATCTGGCAGTTCTGCGTAGCCTGCTGGATCGT
GCACTGCAGCCGCTGGAAAATTTTGATGGTTTTGATCGCATTGATCAGTT
TCAGACCAGCGCAATGCGTTATCAGTGCAATTTTATGCAGTATGCACTGG
CAACCGCACAGCTGCATGCAACCCCGAGCTTTCATGGTTATCTGAGCGCA
GCACAGCGTAATCTGATTGATAAACTGACCCTGCCTGCAGTTTGGCGTTA
TTGGGCATTTGAACAGACCTGGGGTAATCTGAGCCTGGATTGGGATCCGA
TGAAACGTGATAACATTATGCTGAGCGGTTATCTGGGTATGATGCTGGGA
GCATATGAAAGCAATACCGGTGATGATCGTTATCGTCGTTCAGGTGCCCT
GCCGTTTCGTCTGGGGAAACGTGATTGGCCGTATACCCATGATATGGTTA
GCGCAGCAGTTCATGATAATATGCAGCGTAGCGGTATGACCCTGTTTCCG
TGTGAACCGAATTGGATTTATAGCGCATGTAATATGCCTGCAATTAGCAG
CCTGATGATGAGCGATCGTCTGCATGGCACCCGTTATATTGAAAGCGTTG
GTGAAGATTTTCGTCGTCGCCTGCATGGTGAATTTATCACACCGGATGGT
CGTATTACCGCAATTCGTAGCAGCCGTCTGGGTGTTACCATTCCGATGCT
GACCAGCACCATGGCAGATTGTGGTCTGGCAAGCATGCTGCATGCATTTG
ATCCGGAACTGGCACAGCGTTGTTGGACCATTGCACGTCGCGAATTTATT
GATACCACCGGTCCGGAACCTGTTATTGTTCTGCGTGGTTGGGATGCAAT
TGATACCGGTAATTATCGTAAAACCACCCTGGGTGCAGTTGCACCGGTTA
TGTGGGCAGCAGCAGAAATGGGTGATACCGATCTGGTTGCACAGCTGACC
ACCACACTGGAACGTCATGCACAGCCGACCGAAACCGGTGGTGCACGTTG
GTATGCAGAACTGAGCACCAATATGAATGCAATGGCAGCACTGGCACGTT
TTAATCCTCCGGGTGGCCAGCGTGCACTGATTAGCGCAGGTCCGGGTACA
CAGATTCTGACAGGTCCGGTTCTGGATGATGTTGTTTATCCGGAAGTTCT
GGTTGCGAGCGCACGTACCGATGGTGCCGATCTGCGTCTGGTTCTGCGTC
CGGGTGCCGGTGCAGCACGTGTTAGCATTGGTGTTCGTCATCTGCATCCT
GGTGGTCGTTATCGTGTTAATGGTGCCGTTGATAGCGAAGTTACCGCAGA
TAATCAGGGTCGTAGCCATCTGGAAGTTGATCTGATTGATCGTACCGAAG
TTAGCCTGACTCCGGCACCGTAA
SEQ ID NO: 9:
>LDHOt codon-optimised nucleotide sequence from
putative linalool dehydratase WP_006561625
(Oscillochloris trichoides)
ATGCTGCCGGAACGTCTGACCGCATATCTGCGTTATTGTACCCGTCT
GGCACTGCAGGCACCGAATCGTTGGGATGGTTTTGATCTGCATGCACCGG
ATGCACGTCCGACCGCACTGCGTAATCAGATCTTTTTTGTTGGTTGTGCA
CTGGCAGCCCTGGCACGTCATCCGCATGCAGCACAAGAGGAACGTGCAAT
GGCAGTTGATGCCCTGGCAGATCTGACCGATCGTATGATTCAGCGTCGTG
TTTGGGCAGCATGGGCAACCGAAACCGAACGTACCAGCCTGCGTCCGGAT
CCTGTTGATGCAGGTTATGGCACCTATACCGCACCGCTGGCAATGCTGTT
TGGTCTGCAGGGTGTTCTGGGTGGTCAGGTTCGTTATGGTGAAGATCCGT
TTACCCTGCGTTGGAGCGCAGATGTTCGTAGCTGTTATACCGTTCGTGAA
CTGATTGCAGCACTGGCAAAACAGAGCCAGGATAGTCCGGAAGGTGCAAT
TCGTTGTGAAGGTGATCTGGCAACCCCGAGCGCCATGGCAGCGCTGGTTT
GGGCACTGCGTCTGCATGATCTGGCCTATGCAACCGAATATGGCACCAGC
GGCACCACCTGGCTGAAAACCCTGGGTGAACGTATGGCAATTCGTGGTCC
GCGTCTGTTTAATCGTCATACCCTGGCAGCAGGTTGGAATATTGCAAATC
GTCGTGCAAGCGGTAGTGCAGATGGTCTGGAAGATGCATGGGCACTGGCC
CTGAGCGCTCCGCTGGATCGCGAACTGATCGCAGGCTTGGCAGAACGTTA
TTGGGCAGGCGCAGATAAACTGCGTGAACGTGGTGATGCACTGAGCCTGG
GTTTTAGCTATCTGCTGGCAGTTGAACTGGGTGAAACCCAGCTGGCAGCA
AGCCTGCTGGCAAGCGCAGAACAGCGTTTTGGTTTTGATGAAGATGATGA
ACAGGGTCGTCGCATTAAAGATAGTCCGGTTACCCCGTGGGTTACCGCAC
TGTTTGCAATTGGTGAAGCCGGTGGTATGGCACGTCTGCTGGAAGCAGCA
CTGCCTCCGCTGCCGCAGCCGGAAATTCCTGCACCGGGTTGGCCTGAATG
GCCAGAGTGGCCAGAATGGCCTCCGCTGGATCTGGCAGAACCGCAGCTGG
AACAGGATCAGGCCGAAGAACGTCGTGATCAGGCAGCAGAAGAAGAAGGT
TGTTAA
SEQ ID NO: 10:
>LDHCd without periplasmic tag and with C-His6-
tag (SEQ ID NO: 39), Linalool Dehydratase from
Castellaniella defragrans, codon-optimised
ATGGCCGAACTGCCACCTGGTCGCTTGGCCACGACCGAGGACTATT
TCGCACAACAGGCCAAACAAGCGGTTACTCCGGATGTGATGGCTCAACTG
GCGTACATGAACTATATTGACTTTATCAGCCCCTTCTATTCTCGCGGTTG
TAGCTTTGAGGCTTGGGAACTGAAGCATACCCCACAGCGCGTGATTAAGT
ACAGCATCGCGTTTTACGCTTATGGCCTGGCAAGTGTGGCGCTGATTGAT
CCGAAACTGCGTGCGTTAGCCGGTCATGATCTCGACATTGCGGTGTCGAA
AATGAAGTGCAAACGGGTATGGGGCGATTGGGAGGAAGATGGGTTCGGTA
CCGATCCGATCGAGAAAGAGAACATCATGTACAAAGGCCATTTAAACCTG
ATGTATGGGTTGTACCAGCTCGTAACAGGCAGTCGTCGCTATGAAGCCGA
ACACGCACATCTCACCCGCATCATTCACGATGAGATTGCGGCGAATCCTT
TTGCGGGCATTGTGTGTGAACCGGATAATTACTTCGTTCAGTGCAATTCG
GTGGCGTATTTATCCTTGTGGGTCTATGACCGGCTGCATGGTACTGATTA
CCGTGCTGCAACACGCGCATGGCTGGACTTCATCCAGAAAGACCTGATTG
ACCCGGAACGTGGTGCGTTCTACCTGTCATATCACCCCGAATCTGGCGCA
GTTAAGCCGTGGATTAGCGCGTATACGACAGCCTGGACGTTAGCGATGGT
ACACGGAATGGACCCGGCGTTTTCCGAACGCTATTATCCGCGCTTTAAAC
AGACCTTCGTCGAAGTCTACGACGAAGGCCGTAAAGCCCGTGTTCGCGAA
ACTGCCGGGACGGATGATGCCGATGGTGGCGTTGGTCTGGCATCCGCGTT
TACGCTGCTTCTGGCACGCGAGATGGGCGATCAGCAACTGTTCGATCAGT
TACTTAACCACTTGGAACCGCCCGCCAAACCGAGCATTGTCTCAGCTAGT
CTGCGCTATGAACATCCGGGGTCGTTGCTCTTCGATGAACTGCTGTTTCT
GGCAAAAGTGCATGCGGGCTTTGGTGCCCTGTTACGTATGCCACCTCCGG
CTGCCAAACTGGCGGGCAAACATCATCACCATCACCATTAA
SEQ ID NO: 11:
>gi|403399445|sp|E1XUJ2.1|LDI_CASDE RecName:
Full = Linalool dehydratase/isomerase; AltName:
Full = Geraniol isomerase; AltName: Full =
Linalool dehydratase-isomerase; AltName: Full =
Myrcene hydratase; Flags: Precursor; underlined
is the periplasmic tag.
MRFTLKTTAIVSAAALLAGFGPPPRAAELPPGRLATTEDYFAQQAKQAV
TPDVMAQLAYMNYIDFISPFYSRGCSFEAWELKHTPQRVIKYSIAFYAYG
LASVALIDPKLRALAGHDLDIAVSKMKCKRVWGDWEEDGFGTDPIEKENI
MYKGHLNLMYGLYQLVTGSRRYEAEHANLTRIIHDEIAANPFAGIVCEPD
NYFVQCNSVAYLSLVVVYDRLHGTDYRAATRAWLDFIQKDLIDPERGAFY
LSYHPESGAVKPWISAYTTAWTLAMVHGMDPAFSERYYPRFKQTFVEVYD
EGRKARVRETAGTDDADGGVGLASAFTLLLAREMGDQQLFDQLLNHLEPP
AKPSIVSASLRYEHPGSLLFDELLFLAKVHAGFGALLRMPPPAAKLAGK
SEQ ID NO: 12:
>LDHCd without a periplasmic tag and with C-His₆-
tag (SEQ ID NO: 39), Linalool Dehydratase from
Castellaniella defragrans, codon-optimised (in
Plasmid pPI010)
ATGGCCGAACTGCCACCTGGTCGCTTGGCCACGACCGAGGACTATT
TCGCACAACAGGCCAAACAAGCGGTTACTCCGGATGTGATGGCTCAACTG
GCGTACATGAACTATATTGACTTTATCAGCCCCTTCTATTCTCGCGGTTG
TAGCTTTGAGGCTTGGGAACTGAAGCATACCCCACAGCGCGTGATTAAGT
ACAGCATCGCGTTTTACGCTTATGGCCTGGCAAGTGTGGCGCTGATTGAT
CCGAAACTGCGTGCGTTAGCCGGTCATGATCTCGACATTGCGGTGTCGAA
AATGAAGTGCAAACGGGTATGGGGCGATTGGGAGGAAGATGGGTTCGGTA
CCGATCCGATCGAGAAAGAGAACATCATGTACAAAGGCCATTTAAACCTG
ATGTATGGGTTGTACCAGCTCGTAACAGGCAGTCGTCGCTATGAAGCCGA
ACACGCACATCTCACCCGCATCATTCACGATGAGATTGCGGCGAATCCTT
TTGCGGGCATTGTGTGTGAACCGGATAATTACTTCGTTCAGTGCAATTCG
GTGGCGTATTTATCCTTGTGGGTCTATGACCGGCTGCATGGTACTGATTA
CCGTGCTGCAACACGCGCATGGCTGGACTTCATCCAGAAAGACCTGATTG
ACCCGGAACGTGGTGCGTTCTACCTGTCATATCACCCCGAATCTGGCGCA
GTTAAGCCGTGGATTAGCGCGTATACGACAGCCTGGACGTTAGCGATGGT
ACACGGAATGGACCCGGCGTTTTCCGAACGCTATTATCCGCGCTTTAAAC
AGACCTTCGTCGAAGTCTACGACGAAGGCCGTAAAGCCCGTGTTCGCGAA
ACTGCCGGGACGGATGATGCCGATGGTGGCGTTGGTCTGGCATCCGCGTT
TACGCTGCTTCTGGCACGCGAGATGGGCGATCAGCAACTGTTCGATCAGT
TACTTAACCACTTGGAACCGCCCGCCAAACCGAGCATTGTCTCAGCTAGT
CTGCGCTATGAACATCCGGGGTCGTTGCTCTTCGATGAACTGCTGTTTCT
GGCAAAAGTGCATGCGGGCTTTGGTGCCCTGTTACGTATGCCACCTCCGG
CTGCCAAACTGGCGGGCAAACATCATCACCATCACCATTAA
SEQ ID NO: 13:
>Protein sequence of the C. defragrans linalool
dehydratase without a periplasmic tag including
a C-His6-tag (SEQ ID NO: 39)
(as in plasmid ID pPP010)
MAELPPGRLATTEDYFAQQAKQAVTPDVMAQLAYMNYIDFISPFYSRGC
SFEAWELKHTPQRVIKYSIAFYAYGLASVALIDPKLRALAGHDLDIAVSK
MKCKRVWGDWEEDGFGTDPIEKENIMYKGHLNLMYGLYQLVTGSRRYEAE
HAHLTRIIHDEIAANPFAGIVCEPDNYFVQCNSVAYLSLWVYDRLHGTDY
RAATRAWLDFIQKDLIDPERGAFYLSYHPESGAVKPWISAYTTAWTLAMV
HGMDPAFSERYYPRFKQTFVEVYDEGRKARVRETAGTDDADGGVGLASAF
TLLLAREMGDQQLFDQLLNHLEPPAKPSIVSASLRYEHPGSLLFDELLFL
AKVHAGFGALLRMPPPAAKLAGKHHHHHH
SEQ ID NO: 14:
>LDHCd1-B11 (LDHCd variant H83A, part of plasmid
ID pPI011)
MAELPPGRLATTEDYFAQQAKQAVTPDVMAQLAYMNYIDFISPFYSRGCS
FEAWELKATPQRVIKYSIAFYAYGLASVALIDPKLRALAGHDLDIAVSKM
KCKRVWGDWEEDGFGTDPIEKENIMYKGHLNLMYGLYQLVTGSRRYEAEH
AHLTRIIHDEIAANPFAGIVCEPDNYFVQCNSVAYLSLVVVYDRLHGTDY
RAATRAWLDFIQKDLIDPERGAFYLSYHPESGAVKPWISAYTTAWTLAMV
HGMDPAFSERYYPRFKQTFVEVYDEGRKARVRETAGTDDADGGVGLASAF
TLLLAREMGDQQLFDQLLNHLEPPAKPSIVSASLRYEHPGSLLFDELLFL
AKVHAGFGALLRMPPPAAKLAGKHHHHHH
SEQ ID NO: 15:
>LDHCd1-G7 (LDHCd variant H252A, part of plasmid
ID pPI012)
MAELPPGRLATTEDYFAQQAKQAVTPDVMAQLAYMNYIDFISPFYSRGCS
FEAWELKHTPQRVIKYSIAFYAYGLASVALIDPKLRALAGHDLDIAVSKM
KCKRVWGDWEEDGFGTDPIEKENIMYKGHLNLMYGLYQLVTGSRRYEAEH
AHLTRIIHDEIAANPFAGIVCEPDNYFVQCNSVAYLSLVVVYDRLHGTDY
RAATRAWLDFIQKDLIDPERGAFYLSYAPESGAVKPWISAYTTAWTLAMV
HGMDPAFSERYYPRFKQTFVEVYDEGRKARVRETAGTDDADGGVGLASAF
TLLLAREMGDQQLFDQLLNHLEPPAKPSIVSASLRYEHPGSLLFDELLFL
AKVHAGFGALLRMPPPAAKLAGKHHHHHH
SEQ ID NO: 16:
>LDHCd2-D5 (LDHCd variant H83A, R169S, part of
plasmid ID pPI015)
MAELPPGRLATTEDYFAQQAKQAVTPDVMAQLAYMNYIDFISPFYSRGCS
FEAWELKATPQRVIKYSIAFYAYGLASVALIDPKLRALAGHDLDIAVSKM
KCKRVWGDWEEDGFGTDPIEKENIMYKGHLNLMYGLYQLVTGSSRYEAEH
AHLTRIIHDEIAANPFAGIVCEPDNYFVQCNSVAYLSLVVVYDRLHGTDY
RAATRAWLDFIQKDLIDPERGAFYLSYHPESGAVKPWISAYTTAWTLAMV
HGMDPAFSERYYPRFKQTFVEVYDEGRKARVRETAGTDDADGGVGLASAF
TLLLAREMGDQQLFDQLLNHLEPPAKPSIVSASLRYEHPGSLLFDELLFL
AKVHAGFGALLRMPPPAAKLAGKHHHHHH
SEQ ID NO: 17:
>LDHCd2-E5 (LDHCd variant H83A, R169G, part of
plasmid ID pPI016)
MAELPPGRLATTEDYFAQQAKQAVTPDVMAQLAYMNYIDFISPFYSRGCS
FEAWELKATPQRVIKYSIAFYAYGLASVALIDPKLRALAGHDLDIAVSKM
KCKRVWGDWEEDGFGTDPIEKENIMYKGHLNLMYGLYQLVTGSGRYEAEH
AHLTRIIHDEIAANPFAGIVCEPDNYFVQCNSVAYLSLVVVYDRLHGTDY
RAATRAWLDFIQKDLIDPERGAFYLSYHPESGAVKPWISAYTTAWTLAMV
HGMDPAFSERYYPRFKQTFVEVYDEGRKARVRETAGTDDADGGVGLASAF
TLLLAREMGDQQLFDQLLNHLEPPAKPSIVSASLRYEHPGSLLFDELLFL
AKVHAGFGALLRMPPPAAKLAGKHHHHHH
SEQ ID NO: 18:
>LDHCd2-G6 (LDHCd variant H83A, I186C, part of
plasmid ID pPI018)
MAELPPGRLATTEDYFAQQAKQAVTPDVMAQLAYMNYIDFISPFYSRGCS
FEAWELKATPQRVIKYSIAFYAYGLASVALIDPKLRALAGHDLDIAVSKM
KCKRVWGDWEEDGFGTDPIEKENIMYKGHLNLMYGLYQLVTGSRRYEAEH
AHLTRIIHDECAANPFAGIVCEPDNYFVQCNSVAYLSLVVVYDRLHGTDY
RAATRAWLDFIQKDLIDPERGAFYLSYHPESGAVKPWISAYTTAVVTLAM
VHGMDPAFSERYYPRFKQTFVEVYDEGRKARVRETAGTDDADGGVGLASA
FTLLLAREMGDQQLFDQLLNHLEPPAKPSIVSASLRYEHPGSLLFDELLF
LAKVHAGFGALLRMPPPAAKLAGKHHHHHH
SEQ ID NO: 19:
>LDHCd2-F9 (LDHCd variant H83A, R359S, part of
plasmid ID pPI020)
MAELPPGRLATTEDYFAQQAKQAVTPDVMAQLAYMNYIDFISPFYSRGCS
FEAWELKATPQRVIKYSIAFYAYGLASVALIDPKLRALAGHDLDIAVSKM
KCKRVWGDWEEDGFGTDPIEKENIMYKGHLNLMYGLYQLVTGSRRYEAEH
AHLTRIIHDEIAANPFAGIVCEPDNYFVQCNSVAYLSLVVVYDRLHGTDY
RAATRAWLDFIQKDLIDPERGAFYLSYHPESGAVKPWISAYTTAWTLAMV
HGMDPAFSERYYPRFKQTFVEVYDEGRKARVRETAGTDDADGGVGLASAF
TLLLAREMGDQQLFDQLLNHLEPPAKPSIVSASLSYEHPGSLLFDELLFL
AKVHAGFGALLRMPPPAAKLAGKHHHHHH
SEQ ID NO: 20:
>LDHCd2-G9 (LDHCd variant H83A, R359L, part of
plasmid ID pPI021)
MAELPPGRLATTEDYFAQQAKQAVTPDVMAQLAYMNYIDFISPFYSRGCS
FEAWELKATPQRVIKYSIAFYAYGLASVALIDPKLRALAGHDLDIAVSKM
KCKRVWGDWEEDGFGTDPIEKENIMYKGHLNLMYGLYQLVTGSRRYEAEH
AHLTRIIHDEIAANPFAGIVCEPDNYFVQCNSVAYLSLVVVYDRLHGTDY
RAATRAWLDFIQKDLIDPERGAFYLSYHPESGAVKPWISAYTTAWTLAMV
HGMDPAFSERYYPRFKQTFVEVYDEGRKARVRETAGTDDADGGVGLASAF
TLLLAREMGDQQLFDQLLNHLEPPAKPSIVSASLLYEHPGSLLFDELLFL
AKVHAGFGALLRMPPPAAKLAGKHHHHHH
SEQ ID NO: 21:
>LDHCd3-G5 (LDHCd variant R169H, part of plasmid
ID pPI022)
MAELPPGRLATTEDYFAQQAKQAVTPDVMAQLAYMNYIDFISPFYSRGCS
FEAWELKHTPQRVIKYSIAFYAYGLASVALIDPKLRALAGHDLDIAVSKM
KCKRVWGDWEEDGFGTDPIEKENIMYKGHLNLMYGLYQLVTGSHRYEAEH
AHLTRIIHDEIAANPFAGIVCEPDNYFVQCNSVAYLSLVVVYDRLHGTDY
RAATRAWLDFIQKDLIDPERGAFYLSYHPESGAVKPWISAYTTAWTLAMV
HGMDPAFSERYYPRFKQTFVEVYDEGRKARVRETAGTDDADGGVGLASAF
TLLLAREMGDQQLFDQLLNHLEPPAKPSIVSASLRYEHPGSLLFDELLFL
AKVHAGFGALLRMPPPAAKLAGKHHHHHH
SEQ ID NO: 22:
>LDHCd3-H5 (LDHCd variant R169D, part of plasmid
ID pPI023)
MAELPPGRLATTEDYFAQQAKQAVTPDVMAQLAYMNYIDFISPFYSRGCS
FEAWELKHTPQRVIKYSIAFYAYGLASVALIDPKLRALAGHDLDIAVSKM
KCKRVWGDWEEDGFGTDPIEKENIMYKGHLNLMYGLYQLVTGSDRYEAEH
AHLTRIIHDEIAANPFAGIVCEPDNYFVQCNSVAYLSLVVVYDRLHGTDY
RAATRAWLDFIQKDLIDPERGAFYLSYHPESGAVKPWISAYTTAWTLAMV
HGMDPAFSERYYPRFKQTFVEVYDEGRKARVRETAGTDDADGGVGLASAF
TLLLAREMGDQQLFDQLLNHLEPPAKPSIVSASLRYEHPGSLLFDELLFL
AKVHAGFGALLRMPPPAAKLAGKHHHHHH
SEQ ID NO: 23:
>LDHCd3-C6 (LDHCd variant I186M, part of plasmid
ID pPI024)
MAELPPGRLATTEDYFAQQAKQAVTPDVMAQLAYMNYIDFISPFYSRGCS
FEAWELKHTPQRVIKYSIAFYAYGLASVALIDPKLRALAGHDLDIAVSKM
KCKRVWGDWEEDGFGTDPIEKENIMYKGHLNLMYGLYQLVTGSRRYEAEH
AHLTRIIHDEMAANPFAGIVCEPDNYFVQCNSVAYLSLVVVYDRLHGTDY
RAATRAWLDFIQKDLIDPERGAFYLSYHPESGAVKPWISAYTTAVVTLAM
VHGMDPAFSERYYPRFKQTFVEVYDEGRKARVRETAGTDDADGGVGLASA
FTLLLAREMGDQQLFDQLLNHLEPPAKPSIVSASLRYEHPGSLLFDELLF
LAKVHAGFGALLRMPPPAAKLAGKHHHHHH
SEQ ID NO: 24:
>LDHCd3-G9 (LDHCd variant R359S, part of plasmid
ID pPI025)
MAELPPGRLATTEDYFAQQAKQAVTPDVMAQLAYMNYIDFISPFYSRGCS
FEAWELKHTPQRVIKYSIAFYAYGLASVALIDPKLRALAGHDLDIAVSKM
KCKRVWGDWEEDGFGTDPIEKENIMYKGHLNLMYGLYQLVTGSRRYEAEH
AHLTRIIHDEIAANPFAGIVCEPDNYFVQCNSVAYLSLVVVYDRLHGTDY
RAATRAWLDFIQKDLIDPERGAFYLSYHPESGAVKPWISAYTTAWTLAMV
HGMDPAFSERYYPRFKQTFVEVYDEGRKARVRETAGTDDADGGVGLASAF
TLLLAREMGDQQLFDQLLNHLEPPAKPSIVSASLSYEHPGSLLFDELLFL
AKVHAGFGALLRMPPPAAKLAGKHHHHHH
SEQ ID NO: 25:
>LDHCd1-A4 (LDHCd variant A58R, part of plasmid
ID pPI026)
MAELPPGRLATTEDYFAQQAKQAVTPDVMAQLRYMNYIDFISPFYSRGCS
FEAWELKHTPQRVIKYSIAFYAYGLASVALIDPKLRALAGHDLDIAVSKM
KCKRVWGDWEEDGFGTDPIEKENIMYKGHLNLMYGLYQLVTGSRRYEAEH
AHLTRIIHDEIAANPFAGIVCEPDNYFVQCNSVAYLSLVVVYDRLHGTDY
RAATRAWLDFIQKDLIDPERGAFYLSYHPESGAVKPWISAYTTAVVTLAM
VHGMDPAFSERYYPRFKQTFVEVYDEGRKARVRETAGTDDADGGVGLASA
FTLLLAREMGDQQLFDQLLNHLEPPAKPSIVSASLRYEHPGSLLFDELLF
LAKVHAGFGALLRMPPPAAKLAGKHHHHHH
SEQ ID NO: 26:
>LDHCd1-A5 (LDHCd variant Y59A, part of plasmid
ID pPI027)
MAELPPGRLATTEDYFAQQAKQAVTPDVMAQLAAMNYIDFISPFYSRGCS
FEAWELKHTPQRVIKYSIAFYAYGLASVALIDPKLRALAGHDLDIAVSKM
KCKRVWGDWEEDGFGTDPIEKENIMYKGHLNLMYGLYQLVTGSRRYEAEH
AHLTRIIHDEIAANPFAGIVCEPDNYFVQCNSVAYLSLVVVYDRLHGTDY
RAATRAWLDFIQKDLIDPERGAFYLSYHPESGAVKPWISAYTTAWTLAMV
HGMDPAFSERYYPRFKQTFVEVYDEGRKARVRETAGTDDADGGVGLASAF
TLLLAREMGDQQLFDQLLNHLEPPAKPSIVSASLRYEHPGSLLFDELLFL
AKVHAGFGALLRMPPPAAKLAGKHHHHHH
SEQ ID NO: 27:
>LDHCd1-B12 (LDHCd variant T84E, part of plasmid )
ID pPI028
MAELPPGRLATTEDYFAQQAKQAVTPDVMAQLAYMNYIDFISPFYSRGCS
FEAWELKHEPQRVIKYSIAFYAYGLASVALIDPKLRALAGHDLDIAVSKM
KCKRVWGDWEEDGFGTDPIEKENIMYKGHLNLMYGLYQLVTGSRRYEAEH
AHLTRIIHDEIAANPFAGIVCEPDNYFVQCNSVAYLSLVVVYDRLHGTDY
RAATRAWLDFIQKDLIDPERGAFYLSYHPESGAVKPWISAYTTAWTLAMV
HGMDPAFSERYYPRFKQTFVEVYDEGRKARVRETAGTDDADGGVGLASAF
TLLLAREMGDQQLFDQLLNHLEPPAKPSIVSASLRYEHPGSLLFDELLFL
AKVHAGFGALLRMPPPAAKLAGKHHHHHH
SEQ ID NO:28:
>LDHCd1-C7 (LDHCd variant I93L, part of plasmid
ID pPI029)
MAELPPGRLATTEDYFAQQAKQAVTPDVMAQLAYMNYIDFISPFYSRGCS
FEAWELKHTPQRVIKYSLAFYAYGLASVALIDPKLRALAGHDLDIAVSKM
KCKRVWGDWEEDGFGTDPIEKENIMYKGHLNLMYGLYQLVTGSRRYEAEH
AHLTRIIHDEIAANPFAGIVCEPDNYFVQCNSVAYLSLVVVYDRLHGTDY
RAATRAWLDFIQKDLIDPERGAFYLSYHPESGAVKPWISAYTTAWTLAMV
HGMDPAFSERYYPRFKQTFVEVYDEGRKARVRETAGTDDADGGVGLASAF
TLLLAREMGDQQLFDQLLNHLEPPAKPSIVSASLRYEHPGSLLFDELLFL
AKVHAGFGALLRMPPPAAKLAGKHHHHHH
SEQ ID NO: 29:
>LDHCd1-D3 (LDHCd variant K109A, part of plasmid
ID pP1030)
MAELPPGRLATTEDYFAQQAKQAVTPDVMAQLAYMNYIDFISPFYSRGCS
FEAWELKHTPQRVIKYSIAFYAYGLASVALIDPALRALAGHDLDIAVSKM
KCKRVWGDWEEDGFGTDPIEKENIMYKGHLNLMYGLYQLVTGSRRYEAEH
AHLTRIIHDEIAANPFAGIVCEPDNYFVQCNSVAYLSLVVVYDRLHGTDY
RAATRAWLDFIQKDLIDPERGAFYLSYHPESGAVKPWISAYTTAWTLAMV
HGMDPAFSERYYPRFKQTFVEVYDEGRKARVRETAGTDDADGGVGLASAF
TLLLAREMGDQQLFDQLLNHLEPPAKPSIVSASLRYEHPGSLLFDELLFL
AKVHAGFGALLRMPPPAAKLAGKHHHHHH
SEQ ID NO: 30:
>LDHCd1-D7 (LDHCd variant V122I, part of plasmid
ID pPI031)
MAELPPGRLATTEDYFAQQAKQAVTPDVMAQLAYMNYIDFISPFYSRGCS
FEAWELKHTPQRVIKYSIAFYAYGLASVALIDPKLRALAGHDLDIAISKM
KCKRVWGDWEEDGFGTDPIEKENIMYKGHLNLMYGLYQLVTGSRRYEAEH
AHLTRIIHDEIAANPFAGIVCEPDNYFVQCNSVAYLSLWVYDRLHGTDYR
AATRAWLDFIQKDLIDPERGAFYLSYHPESGAVKPWISAYTTAWTLAMVH
GMDPAFSERYYPRFKQTFVEVYDEGRKARVRETAGTDDADGGVGLASAFT
LLLAREMGDQQLFDQLLNHLEPPAKPSIVSASLRYEHPGSLLFDELLFLA
KVHAGFGALLRMPPPAAKLAGKHHHHHH
SEQ ID NO: 31:
>LDHCd1-E2 (LDHCd variant D137R, part of plasmid
ID pPI032)
MAELPPGRLATTEDYFAQQAKQAVTPDVMAQLAYMNYIDFISPFYSRGCS
FEAWELKHTPQRVIKYSIAFYAYGLASVALIDPKLRALAGHDLDIAVSKM
KCKRVWGDWEERGFGTDPIEKENIMYKGHLNLMYGLYQLVTGSRRYEAEH
AHLTRIIHDEIAANPFAGIVCEPDNYFVQCNSVAYLSLVVVYDRLHGTDY
RAATRAWLDFIQKDLIDPERGAFYLSYHPESGAVKPWISAYTTAWTLAMV
HGMDPAFSERYYPRFKQTFVEVYDEGRKARVRETAGTDDADGGVGLASAF
TLLLAREMGDQQLFDQLLNHLEPPAKPSIVSASLRYEHPGSLLFDELLFL
AKVHAGFGALLRMPPPAAKLAGKHHHHHH
SEQ ID NO: 32:
>LDHCd1-E8 (LDHCd variant S168D, part of plasmid
ID pPI033)
MAELPPGRLATTEDYFAQQAKQAVTPDVMAQLAYMNYIDFISPFYSRGCS
FEAWELKHTPQRVIKYSIAFYAYGLASVALIDPKLRALAGHDLDIAVSKM
KCKRVWGDWEEDGFGTDPIEKENIMYKGHLNLMYGLYQLVTGDRRYEAEH
AHLTRIIHDEIAANPFAGIVCEPDNYFVQCNSVAYLSLVVVYDRLHGTDY
RAATRAWLDFIQKDLIDPERGAFYLSYHPESGAVKPWISAYTTAWTLAMV
HGMDPAFSERYYPRFKQTFVEVYDEGRKARVRETAGTDDADGGVGLASAF
TLLLAREMGDQQLFDQLLNHLEPPAKPSIVSASLRYEHPGSLLFDELLFL
AKVHAGFGALLRMPPPAAKLAGKHHHHHH
SEQ ID NO: 33:
>LDHCd1-E9 (LDHCd variant R169D, part of plasmid
ID pPI034)
MAELPPGRLATTEDYFAQQAKQAVTPDVMAQLAYMNYIDFISPFYSRGCS
FEAWELKHTPQRVIKYSIAFYAYGLASVALIDPKLRALAGHDLDIAVSKM
KCKRVWGDWEEDGFGTDPIEKENIMYKGHLNLMYGLYQLVTGSDRYEAEH
AHLTRIIHDEIAANPFAGIVCEPDNYFVQCNSVAYLSLVVVYDRLHGTDY
RAATRAWLDFIQKDLIDPERGAFYLSYHPESGAVKPWISAYTTAWTLAMV
HGMDPAFSERYYPRFKQTFVEVYDEGRKARVRETAGTDDADGGVGLASAF
TLLLAREMGDQQLFDQLLNHLEPPAKPSIVSASLRYEHPGSLLFDELLFL
AKVHAGFGALLRMPPPAAKLAGKHHHHHH
SEQ ID NO: 34:
>LDHCd1-F9 (LDHCd variant D199N, part of plasmid
ID pPI035)
MAELPPGRLATTEDYFAQQAKQAVTPDVMAQLAYMNYIDFISPFYSRGCS
FEAWELKHTPQRVIKYSIAFYAYGLASVALIDPKLRALAGHDLDIAVSKM
KCKRVWGDWEEDGFGTDPIEKENIMYKGHLNLMYGLYQLVTGSRRYEAEH
AHLTRIIHDEIAANPFAGIVCEPNNYFVQCNSVAYLSLVVVYDRLHGTDY
RAATRAWLDFIQKDLIDPERGAFYLSYHPESGAVKPWISAYTTAWTLAMV
HGMDPAFSERYYPRFKQTFVEVYDEGRKARVRETAGTDDADGGVGLASAF
TLLLAREMGDQQLFDQLLNHLEPPAKPSIVSASLRYEHPGSLLFDELLFL
AKVHAGFGALLRMPPPAAKLAGKHHHHHH
SEQ ID NO: 35:
>LDHCd1-G5 (LDHCd variant A230E, part of plasmid
ID pP1036)
MAELPPGRLATTEDYFAQQAKQAVTPDVMAQLAYMNYIDFISPFYSRGCS
FEAWELKHTPQRVIKYSIAFYAYGLASVALIDPKLRALAGHDLDIAVSKM
KCKRVWGDWEEDGFGTDPIEKENIMYKGHLNLMYGLYQLVTGSRRYEAEH
AHLTRIIHDEIAANPFAGIVCEPDNYFVQCNSVAYLSLVVVYDRLHGTDY
RAATREWLDFIQKDLIDPERGAFYLSYHPESGAVKPWISAYTTAWTLAMV
HGMDPAFSERYYPRFKQTFVEVYDEGRKARVRETAGTDDADGGVGLASAF
TLLLAREMGDQQLFDQLLNHLEPPAKPSIVSASLRYEHPGSLLFDELLF
LAKVHAGFGALLRMPPPAAKLAGKHHHHHH
SEQ ID NO: 36:
>LDHCd1-H6 (LDHCd variant L366V, part of plasmid
ID pPI037)
MAELPPGRLATTEDYFAQQAKQAVTPDVMAQLAYMNYIDFISPFYSRGCS
FEAWELKHTPQRVIKYSIAFYAYGLASVALIDPKLRALAGHDLDIAVSKM
KCKRVWGDWEEDGFGTDPIEKENIMYKGHLNLMYGLYQLVTGSRRYEAEH
AHLTRIIHDEIAANPFAGIVCEPDNYFVQCNSVAYLSLVVVYDRLHGTDY
RAATRAWLDFIQKDLIDPERGAFYLSYHPESGAVKPWISAYTTAWTLAMV
HGMDPAFSERYYPRFKQTFVEVYDEGRKARVRETAGTDDADGGVGLASAF
TLLLAREMGDQQLFDQLLNHLEPPAKPSIVSASLRYEHPGSVLFDELLFL
AKVHAGFGALLRMPPPAAKLAGKHHHHHH
SEQ ID NO: 37:
SEQ ID NO: 13 without the C-terminal His-tag
(cytoplasmic LDHCd without His-tag)
SEQ ID NO: 38:
SEQ ID NO: 11 plus C-terminal His-Tag
SEQ ID NO: 42:
A230E WITHOUT COMPLETE PERIPLASMIC LEADER SEQUENCE
SEQ ID NO: 45:
L366V WITHOUT COMPLETE WITHOUT COMPLETE
PERIPLASMIC LEADER SEQUENCE.
SEQ ID NO: 48:
S168D WITHOUT COMPLETE PERIPLASMICLEADER SEQUENCE.

1. A polypeptide comprising an amino acid sequence with at least 90% amino acid sequence homology to SEQ ID NO:11 or SEQ ID NO:51, wherein said amino acid sequence comprises at least 1-3 alteration(s) relative to SEQ ID NO:11 or SEQ ID NO:51 independently selected from:
a substitution of the amino acid that occupies position 230 with a different amino acid selected from E and equivalent amino acids;
a substitution of the amino acid that occupies position 366 with a different amino acid selected from V and equivalent amino acids; and
a substitution of the amino acid that occupies position 168 with a different amino acid selected from D and equivalent amino acids;
wherein the polypeptide optionally comprises at least one mutation selected from A230E, L366V, and S168D; preferably comprises the amino acid sequence of the A230E variant of SEQ ID NO:42, the amino acid sequence of the L366V variant of SEQ ID NO:45, or the amino acid sequence of the S168D variant of SEQ ID NO:48.

2.-18. (canceled)

19. The polypeptide according to claim 1, wherein the polypeptide has a specific activity in the catalysis of the dehydration of 3-buten-2-ol to 1,3-butadiene that is increased about 1.5 fold or greater when compared to that of a polypeptide consisting of SEQ ID NO: 11, 13, 37, or 38, preferably about 2 fold or greater when compared to that of a polypeptide consisting of SEQ ID NO: 11, 13, 37, or 38, preferably about 2.5 fold or greater when compared to that of a polypeptide consisting of SEQ ID NO: 11, 13, 37, or 38, preferably about 3 fold or greater when compared to that of a polypeptide consisting of SEQ ID NO: 11, 13, 37, or 38, preferably about 3.5 fold or greater when compared to the parent LDH to that of a polypeptide consisting of SEQ ID NO: 11, 13, 37, or 38, preferably about 4 fold or greater when compared to that of a polypeptide consisting of SEQ ID NO: 11, 13, 37, or 38, preferably about 4.5 fold or greater when compared to that of a polypeptide consisting of SEQ ID NO: 11, 13, 37, or 38, or preferably about 5 fold or greater when compared to that of a polypeptide consisting of SEQ ID NO: 11, 13, 37, or 38, and wherein the increased specific activity is observed in at least one specific activity assay.

20.-24. (canceled)

25. A polypeptide comprising an amino acid sequence with at least 90% amino acid sequence homology to SEQ ID NO:11 or SEQ ID NO:51, wherein said amino acid sequence comprises at least 1-3 alteration(s) relative to SEQ ID NO:11 or SEQ ID NO:51 independently selected from:
a substitution of the amino acid that occupies position 58 with a different amino acid selected from R and equivalent amino acids;
a substitution of the amino acid that occupies position 83 with a different amino acid selected from A and equivalent amino acids; and
a substitution of the amino acid that occupies position 252 with a different amino acid selected from A and equivalent amino acids.

26.-42. (canceled)

43. The polypeptide according to claim 25, wherein the polypeptide has a solubility that is increased about 1.5 fold or greater when compared to that of a polypeptide consisting of SEQ ID NO:11, 13, 37, or 38, preferably about 2 fold or greater when compared to that of a polypeptide consisting of SEQ ID NO: 11, 13, 37, or 38, preferably about 2.5 or greater when compared to that of a polypeptide consisting of SEQ ID NO: 11, 13, 37, or 38, preferably about 3 fold or greater when compared to that of a polypeptide consisting of SEQ ID NO: 11, 13, 37, or 38, preferably about 3.5 or greater when compared to that of a polypeptide consisting of SEQ ID NO: 11, 13, 37, or 38, preferably about 4 fold or greater when compared to that of a polypeptide consisting of SEQ ID NO: 11, 13, 37, or 38, preferably about 4.5 fold or greater when compared to that of a polypeptide consisting of SEQ ID NO: 11, 13, 37, or 38, or preferably about 5 fold or greater when compared to that of a polypeptide consisting of SEQ ID NO: 11, 13, 37, or 38, and wherein the increased solubility is observed in at least one solubility assay.

44.-49. (canceled)

50. The polypeptide according to claim 25, wherein said polypeptide has a specific activity in the catalysis of the dehydration of 3-buten-2-ol to 1,3-butadiene that is increased about 1.5 fold or greater when compared to that of a polypeptide consisting of SEQ ID NO: 11, 13, 37, or 38, preferably about 2 fold or greater when compared to that of a polypeptide consisting of SEQ ID NO: 11, 13, 37, or 38, preferably about 2.5 fold or greater when compared to that of a polypeptide consisting of SEQ ID NO: 11, 13, 37, or 38, preferably about 3 fold or greater when compared to that of a polypeptide consisting of SEQ ID NO: 11, 13, 37, or 38, preferably about 3.5 fold or greater when compared to the parent LDH to that of a polypeptide consisting of SEQ ID NO: 11, 13, 37, or 38, preferably about 4 fold or greater when compared to that of a polypeptide consisting of SEQ ID NO: 11, 13, 37, or 38, preferably about 4.5 fold or greater when compared to that of a polypeptide consisting of SEQ ID NO: 11, 13, 37, or 38, or preferably about 5 fold or greater when compared to that of a polypeptide consisting of SEQ ID NO: 11, 13, 37, or 38, and wherein the increased specific activity is observed in at least one specific activity assay.

51. A polypeptide comprising an amino acid sequence with at least 90% amino acid sequence homology to SEQ ID NO:11 or SEQ ID NO:51, wherein said amino acid sequence comprises at least 1-4 alteration(s) relative to SEQ ID NO:11 or SEQ ID NO:51 independently selected from:
a substitution of the amino acid that occupies position 83 with a different amino acid selected from A and equivalent amino acids;
a substitution of the amino acid that occupies position 169 with a different amino acid selected from S, G, H, D and equivalent amino acids;
a substitution of the amino acid that occupies position 186 with a different amino acid selected from C, M and equivalent amino acids; and
a substitution of the amino acid that occupies position 359 with a different amino acid selected from S, L and equivalent amino acids.

52.-83. (canceled)

84. The polypeptide according to claim 51, wherein the polypeptide has a specific activity in the catalysis of the dehydration of linalool to myrcene that is increased about 1.5 fold or greater when compared to that of a polypeptide consisting of SEQ ID NO: 11, 13, 37, or 38, preferably about 2 fold or greater when compared to that of a polypeptide consisting of SEQ ID NO: 11, 13, 37, or 38, preferably about 2.5 or greater when compared to that of a polypeptide consisting of SEQ ID NO: 11, 13, 37, or 38, preferably about 3 fold or greater when compared to that of a polypeptide consisting of SEQ ID NO: 11, 13, 37, or 38, preferably about 3.5 fold or greater when compared to that of a polypeptide consisting of SEQ ID NO: 11, 13, 37, or 38, preferably about 4 fold or greater when compared to that of a polypeptide consisting of SEQ ID NO: 11, 13, 37, or 38, preferably about 4.5 fold or greater when compared to that of a polypeptide consisting of SEQ ID NO: 11, 13, 37, or 38, or preferably about 5 fold or greater when compared to that of a polypeptide consisting of SEQ ID NO: 11, 13, 37, or 38, and wherein the increased solubility is observed in at least one solubility assay.

85.-89. (canceled)

90. A method of using myrcene produced by the polypeptide according to claim 51 in the terpene industry.

91. A method of using myrcene produced by the polypeptide according to claim 51 in the perfume industry for producing a fragrance.

92. A method of using myrcene produced by the polypeptide according to claim 51 in the pharmacological industry.

93. A terpene composition comprising myrcene produced by the polypeptide according to claim 51.

94. A pharmacologic composition comprising myrcene produced by the polypeptide according to claim 51.

95. A fragrance composition comprising myrcene produced by the polypeptide according to claim 51.

96. A sedative composition comprising myrcene produced by the polypeptide according to claim 51.

97. An anti-inflammatory composition comprising myrcene produced by the polypeptide according to claim 51.

98. An analgesic composition comprising myrcene produced by the polypeptide according to claim 51.

99. The polypeptide according to any one of claims 1, 25, and 51, wherein the polypeptide has an activity in the catalysis of the dehydration of 3-methyl-3-buten-2-ol to isoprene that is at least 80% of that of a polypeptide consisting of SEQ ID NO: 11, 13, 37, or 38 (SEQ ID NO. of cyto-cdLD), increased about 1.5 fold or greater when compared to that of a polypeptide consisting of SEQ ID NO: 11, 13, 37, or 38, preferably about 2 fold or greater when compared to that of a polypeptide consisting of SEQ ID NO: 11, 13, 37, or 38, preferably about 2.5 fold or greater when compared to that of a polypeptide consisting of SEQ ID NO: 11, 13, 37, or 38, preferably about 3 fold or greater when compared to that of a polypeptide consisting of SEQ ID NO: 11, 13, 37, or 38, preferably about 3.5 fold or greater when compared to that of a polypeptide consisting of SEQ ID NO: 11, 13, 37, or 38, preferably about 4 fold or greater when compared to that of a polypeptide consisting of SEQ ID NO: 11, 13, 37, or 38, preferably about 4.5 fold or greater when compared to that of a polypeptide consisting of SEQ ID NO: 11, 13, 37, or 38, or preferably about 5 fold or greater when compared to that of a polypeptide consisting of SEQ ID NO: 11, 13, 37, or 38, or preferably about 15 fold or greater when compared to that of a polypeptide consisting of SEQ ID NO: 11, 13, 37, or 38 and wherein said activity is observed in at least one activity assay or preferably about 55 fold or greater when compared to that of a polypeptide consisting of SEQ ID NO: 11, 13, 37, or 38 or preferably about 30 fold or greater when compared to that of a polypeptide consisting of SEQ ID NO: 11, 13, 37, or 38.

100.-125. (canceled)

126. A polynucleotide comprising or consisting essentially of a nucleic acid encoding any one of the polypeptides according to any one of claims 1, 25, and 51, preferably codon-optimized.

127.-129. (canceled)

130. A host cell which is transformed or transduced with the polynucleotide according to claim 126, wherein the polynucleotide is a DNA molecule.

131. (canceled)

132. A microorganism comprising a heterologous DNA molecule encoding the polypeptide according to any one of claims 1, 25, and 51.

133. A transgenic animal or plant comprising a heterologous DNA molecule encoding the polypeptide according to any one of claims 1, 25, and 51.

134.-135. (canceled)

136. A vector comprising the polynucleotide according to claim 126, wherein the polynucleotide is a DNA molecule.

137. A method of producing the polypeptide according to any one of claims 1, 25, and 51, the method comprising:
(i) preparing an expression construct which comprises the polynucleotide of claim 126, with a sequence encoding the polypeptide according to any one of claims 1, 25, and 51 operably linked to one or more regulatory nucleotide sequences; (ii) transfecting or transforming a suitable host cell with the expression construct; (iii) expressing the recombinant polypeptide in said host cell; and (iv) isolating the recombinant polypeptide from said host cell or using the resultant host cell as is or as a cell extract.

138. A method of making a polypeptide with improved solubility and/or improved specific activity in the catalysis of the dehydration of 3-buten-2-ol to 1,3-butadiene and/or specific activity in the catalysis of the dehydration of linalool to myrcene and/or specific activity of the dehydration of 3-methyl-3-buten-2-ol to isoprene, relative to that of a polypeptide consisting of SEQ ID NO: 11, 13, 37, or 38, the method comprising preparing the polypeptide according to any one of claims 1, 25, and 51.

139. A composition comprising one or more polypeptides according to any one of claims 1, 25, and 51.

140.-142. (canceled)

143. The composition according to claim 139, further comprising 3-buten-2-ol and/or linalool and/or 3-methyl-3-buten-2-ol.

144. The composition according to claim 139, further comprising 1,3-butadiene and/or myrcene and/or isoprene.

145. A composition comprising a rubber product polymerized from 1,3-butadiene produced in the presence of the polypeptide according to any one of claims 1, 25, and 51.

146. A composition comprising a copolymer polymerized from 1,3-butadiene produced in the presence of the polypeptide according to any one of claims 1, 25, and 51.

147. A composition comprising a plastic product polymerized from 1,3-butadiene produced in the presence of the polypeptide according to any one of claims 1, 25, and 51.

148. An antibody capable of binding to the polypeptide according to any one of claims 1, 25, and 51.

149. A fusion protein comprising the polypeptide according to any one of claims 1, 25, and 51.

150. A complex comprising the polypeptide according to any one claims 1, 25, and 51, said complex optionally further comprising 3-buten-2-ol, or linalool.

151. A complex comprising the polypeptide according to any one claims 1, 25, and 51, said complex optionally further comprising 3-methyl-3-buten-2-ol.

152. A composition comprising (i) 3-buten-2-ol and a means for producing 1,3-butadiene, and/or (ii) linalool and a means for producing myrcene.

153. A composition comprising (i) a substrate and a means for enzymatically producing 1,3-butadiene from said substrate; and/or (i) a substrate and a means for enzymatically producing myrcene from said substrate.

154. A method of:
(i) producing 1,3-butadiene comprising:
a step for enzymatically converting 3-buten-2-ol to 1,3-butadiene; and
measuring and/or harvesting the 1,3-butadiene thereby produced; and/or
(ii) producing myrcene comprising:
a step for enzymatically converting linalool to myrcene; and
measuring and/or harvesting the myrcene thereby produced.

155.-169. (canceled)

170. Bioderived isoprene, myrcene, and/or 1,3-butadiene having a carbon-12, carbon-13 and carbon-14 isotope ratio that reflects an atmospheric carbon dioxide uptake source, preferably produced by growing the host cell according to claim 130.

171. (canceled)

172. A composition comprising the bioderived isoprene, myrcene, and/or 1,3-butadiene according to claim 170 and a compound other than said bioderived isoprene, myrcene, and/or 1,3-butadiene.

173. (canceled)

174. A biobased polymer comprising the bioderived isoprene, myrcene, and/or 1,3-butadiene according to claim 170.

175. A biobased resin comprising the bioderived isoprene, myrcene, and/or 1,3-butadiene according to claim 170.

176. A molded product obtained by molding the biobased polymer of claim 174.

177. A process for producing the biobased polymer of claim 174 comprising chemically reacting the bioderived isoprene, myrcene, and/or 1,3-butadiene with itself or another compound in a polymer-producing reaction.

178. A molded product obtained by molding the biobased resin of claim 175.

179. A process for producing the biobased resin of claim 175 comprising chemically reacting said bioderived isoprene, myrcene, and/or 1,3-butadiene with itself or another compound in a resin producing reaction.

180. Bioderived isoprene, myrcene, and/or 1,3-butadiene having a carbon-12, carbon-13 and carbon-14 isotope ratio that reflects an atmospheric carbon dioxide uptake source, preferably produced by growing the microorganism according to claim 132.

181. A composition comprising the bioderived isoprene, myrcene, and/or 1,3-butadiene according to claim 180 and a compound other than said bioderived isoprene, myrcene, and/or 1,3-butadiene.

182. A biobased polymer comprising the bioderived isoprene, myrcene, and/or 1,3-butadiene according to claim 180.

183. A biobased resin comprising the bioderived isoprene, myrcene, and/or 1,3-butadiene according to claim 180.

184. A molded product obtained by molding the biobased polymer of claim 182.

185. A process for producing the biobased polymer of claim 182 comprising chemically reacting the bioderived isoprene, myrcene, and/or 1,3-butadiene- with itself or another compound in a polymer-producing reaction.

186. A molded product obtained by molding the biobased resin of claim 183.

187. A process for producing the biobased resin of claim 183 comprising chemically reacting said bioderived isoprene, myrcene, and/or 1,3-butadiene with itself or another compound in a resin producing reaction.
